SEQUENCE LISTING (348 sequences)

SEQ ID NO: 1 - protein, 118 aa, synthetic construct
DVQLQESGPGMVKPSQSLSLTCTVTGYSITSGYDWHWIRHFPGNKLEWMGYISYSGSTNYNPSLKSRISITHDTSKNHFFLKLNSVTTEDTATYYCAREGYSNYFDSWGQGTTLTVSS

SEQ ID NO: 2 - protein, 120 aa, synthetic construct
QVQLQQPGAELVRPGSSVKLSCKASGYTFTSYWMHWVKQRPIQGLEWIGNIDPSDSETHYNQKFKDKATLTVDKSSSTAYMQLSSLTSEDSAVYYCARAGRVFYYAMDYWGQGTSVTVSS

SEQ ID NO: 3 - protein, 119 aa, synthetic construct
EVQLQQSGPVLVKPGPSVKISCKASGFTFTDYYMHWVKQSHGKSLEWIGLVYPYNGGTSYNQKFKGKATLTVDTSSSTAYMELNSLTSEDSAVYYCARRGQRVWYFDVWGTGTTVTVSS

SEQ ID NO: 4 - protein, 118 aa, synthetic construct
QVQLQQSGPELVKPGASVKISCKASGYSFTSYYIHWVKQRPGQGLEWIGWIYPGSGNTKYNEKFKGKATLTADTSSSTAYMQLSSLTSEDSAVYYCARCVLLDYFDYWGQGTTLTVSS

SEQ ID NO: 5 - protein, 114 aa, synthetic construct
QVQLQQSGAELMKPGASVKLSCKATGHTFTGYWIEWVKQRPGHGLEWIGEILPGSGSTNYNEKFKGKATFTADTSSNTAYMQLSSLTTEDSAIYYCAREGFAYWGQGTLVTVSA

SEQ ID NO: 6 - protein, 118 aa, synthetic construct
QIQLVQSGPELKKPGETVKISCKASGYTFTTYGMSWVKQAPGKGLKWMGWINTYSGVPTYADDFKGRFAFSLETSASTAYLQINNLKNEDSATYFCARYYYGSNYDYWGQGTTLTVSS

SEQ ID NO: 7 - protein, 118 aa, synthetic construct
QIQLVQSGPELKKPGETVKISCKASGYTFTTYGMSWVKQAPGKGLKWMGWINTYSGVPTYADDFKGRFAFSLETSASTAYLQINNLKNEDTATYFCARTYYGNYFDYWGQGTTLTVSS

SEQ ID NO: 8 - protein, 118 aa, synthetic construct
QIQLVQSGPELKKPGETVKISCKASGYTFTTYGMSWVKQAPGKGLKWMGWINTYSGVPTYADDFKGRFAFSLETSASTAYLQINNLKNEDTATYFCARFYYGSSFDYWGQGTTLTVSS

SEQ ID NO: 9 - protein, 118 aa, synthetic construct
QVHLQQSGPELVKPGASVKISCKASGYSFTSYYIHWVKQRPGQGLEWIGWIYPRSGNTNYNEKFKDKATLAADTSSSAAYMQLSSLTSEDSAVYYCGRSDFYYGSDYWGQGTTLTVSS

SEQ ID NO: 10 - protein, 122 aa, synthetic construct
EVKLEESGGGLVQPGGSMKLSCVASGFTFSNYWMNWVRQSPEKGLEWVAQIRLKSDNYATHYAESVKGRFTISRDDSKSSVYLQMNNLRAEDTGIYYCTGGYYGSRRGFDYWGQGTTLTVSS

SEQ ID NO: 11 - protein, 107 aa, synthetic construct
SIVMTQTPKFLLVSAGDRVTITCKASQSVSNDVAWYQQKPGQSPKLLIFYASNRYTGVPDRFTGSGYGTDFTFTISTVQAEDLAVYFCQQDYSSPLAFGAGTKLELK

SEQ ID NO: 12 - protein, 106 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLYTFGGGTKLEIK

SEQ ID NO: 13 - protein, 106 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIK

SEQ ID NO: 14 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYASYPYTFGGGTKLEIK

SEQ ID NO: 15 - protein, 107 aa, synthetic construct
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIK

SEQ ID NO: 16 - protein, 107 aa, synthetic construct
DIQMTQSPASLSASVGETVTITCRASENIYSYLAWYQQKQGKSPQLLVNNAKTLAEGVPSRFSGSGSGTQFSLKINSLQPEDFGSYYCQHHYGSPPTFGSGTKLEIK

SEQ ID NO: 17 - protein, 106 aa, synthetic construct; identical to SEQ ID NO: 13

SEQ ID NO: 18 - protein, 107 aa, synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPLTFGAGTKLELK

SEQ ID NO: 19 - protein, 107 aa, synthetic construct
DIQMTQSPSSLSASLGERVSLTCRASQEISGYLGWLQQKPDGTIKRLIYAASTLDSGVPRRFSGSRSGSDYSLTFSSLESEDFADYYCLQYASYPWTFGGGTKLEIK

SEQ ID NO: 20 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 21 - protein, 8 aa, synthetic construct
GYSITSGY

SEQ ID NO: 22 - protein, 7 aa, synthetic construct
GYTFTSY

SEQ ID NO: 23 - protein, 7 aa, synthetic construct
GFTFTDY

SEQ ID NO: 24 - protein, 7 aa, synthetic construct
GYSFTSY

SEQ ID NO: 25 - protein, 7 aa, synthetic construct
GHTFTGY

SEQ ID NO: 26 - protein, 7 aa, synthetic construct
GYTFTTY

SEQ ID NO: 27 - protein, 7 aa, synthetic construct
GFTFSNY

SEQ ID NO: 28 - protein, 6 aa, synthetic construct
SGYDWH

SEQ ID NO: 29 - protein, 5 aa, synthetic construct
SYWMH

SEQ ID NO: 30 - protein, 5 aa, synthetic construct
DYYMH

SEQ ID NO: 31 - protein, 5 aa, synthetic construct
SYYIH

SEQ ID NO: 32 - protein, 5 aa, synthetic construct
GYWIE

SEQ ID NO: 33 - protein, 5 aa, synthetic construct
TYGMS

SEQ ID NO: 34 - protein, 5 aa, synthetic construct
NYWMN

SEQ ID NO: 35 - protein, 9 aa, synthetic construct
GYSITSGYD

SEQ ID NO: 36 - protein, 8 aa, synthetic construct
GYTFTSYW

SEQ ID NO: 37 - protein, 5 aa, synthetic construct
GFTFY

SEQ ID NO: 38 - protein, 8 aa, synthetic construct
GYSFTSYY

SEQ ID NO: 39 - protein, 8 aa, synthetic construct
GHTFTGYW

SEQ ID NO: 40 - protein, 8 aa, synthetic construct
GYTFTTYG

SEQ ID NO: 41 - protein, 8 aa, synthetic construct
GFTFSNYW

SEQ ID NO: 42 - protein, 5 aa, synthetic construct
SYSGS

SEQ ID NO: 43 - protein, 6 aa, synthetic construct
DPSDSE

SEQ ID NO: 44 - protein, 6 aa, synthetic construct
YPYNGG

SEQ ID NO: 45 - protein, 6 aa, synthetic construct
YPGSGN

SEQ ID NO: 46 - protein, 6 aa, synthetic construct
LPGSGS

SEQ ID NO: 47 - protein, 6 aa, synthetic construct
NTYSGV

SEQ ID NO: 48 - protein, 6 aa, synthetic construct
YPRSGN

SEQ ID NO: 49 - protein, 8 aa, synthetic construct
RLKSDNYA

SEQ ID NO: 50 - protein, 16 aa, synthetic construct
YISYSGSTNYNPSLKS

SEQ ID NO: 51 - protein, 17 aa, synthetic construct
NIDPSDSETHYNQKFKD

SEQ ID NO: 52 - protein, 17 aa, synthetic construct
LVYPYNGGTSYNQKFKG

SEQ ID NO: 53 - protein, 17 aa, synthetic construct
WIYPGSGNTKYNEKFKG

SEQ ID NO: 54 - protein, 17 aa, synthetic construct
EILPGSGSTNYNEKFKG

SEQ ID NO: 55 - protein, 17 aa, synthetic construct
WINTYSGVPTYADDFKG

SEQ ID NO: 56 - protein, 17 aa, synthetic construct
WIYPRSGNTNYNEKFKD

SEQ ID NO: 57 - protein, 19 aa, synthetic construct
QIRLKSDNYATHYAESVKG

SEQ ID NO: 58 - protein, 7 aa, synthetic construct
ISYSGST

SEQ ID NO: 59 - protein, 8 aa, synthetic construct
IDPSDSET

SEQ ID NO: 60 - protein, 8 aa, synthetic construct
VYPYNGGT

SEQ ID NO: 61 - protein, 8 aa, synthetic construct
IYPGSGNT

SEQ ID NO: 62 - protein, 8 aa, synthetic construct
ILPGSGST

SEQ ID NO: 63 - protein, 8 aa, synthetic construct
INTYSGVP

SEQ ID NO: 64 - protein, 8 aa, synthetic construct
IYPRSGNT

SEQ ID NO: 65 - protein, 10 aa, synthetic construct
IRLKSDNYAT

SEQ ID NO: 66 - protein, 9 aa, synthetic construct
EGYSNYFDS

SEQ ID NO: 67 - protein, 11 aa, synthetic construct
AGRVFYYAMDY

SEQ ID NO: 68 - protein, 10 aa, synthetic construct
RGQRVWYFDV

SEQ ID NO: 69 - protein, 9 aa, synthetic construct
CVLLDYFDY

SEQ ID NO: 70 - protein, 5 aa, synthetic construct
EGFAY

SEQ ID NO: 71 - protein, 9 aa, synthetic construct
YYYGSNYDY

SEQ ID NO: 72 - protein, 9 aa, synthetic construct
TYYGNYFDY

SEQ ID NO: 73 - protein, 9 aa, synthetic construct
FYYGSSFDY

SEQ ID NO: 74 - protein, 9 aa, synthetic construct
SDFYYGSDY

SEQ ID NO: 75 - protein, 11 aa, synthetic construct
GYYGSRRGFDY

SEQ ID NO: 76 - protein, 11 aa, synthetic construct
AREGYSNYFDS

SEQ ID NO: 77 - protein, 13 aa, synthetic construct
ARAGRVFYYAMDY

SEQ ID NO: 78 - protein, 12 aa, synthetic construct
ARRGQRVWYFDV

SEQ ID NO: 79 - protein, 11 aa, synthetic construct
ARCVLLDYFDY

SEQ ID NO: 80 - protein, 7 aa, synthetic construct
AREGFAY

SEQ ID NO: 81 - protein, 11 aa, synthetic construct
ARYYYGSNYDY

SEQ ID NO: 82 - protein, 11 aa, synthetic construct
ARTYYGNYFDY

SEQ ID NO: 83 - protein, 11 aa, synthetic construct
ARFYYGSSFDY

SEQ ID NO: 84 - protein, 11 aa, synthetic construct
GRSDFYYGSDY

SEQ ID NO: 85 - protein, 13 aa, synthetic construct
TGGYYGSRRGFDY

SEQ ID NO: 86 - protein, 11 aa, synthetic construct
KASQSVSNDVA

SEQ ID NO: 87 - protein, 11 aa, synthetic construct
RASQDISNYLN

SEQ ID NO: 88 - protein, 11 aa, synthetic construct
KASQDINKYIA

SEQ ID NO: 89 - protein, 11 aa, synthetic construct
RASQEISGYLS

SEQ ID NO: 90 - protein, 11 aa, synthetic construct
KASQDVGIAVA

SEQ ID NO: 91 - protein, 11 aa, synthetic construct
RASENIYSYLA

SEQ ID NO: 92 - protein, 11 aa, synthetic construct
RASQEISGYLG

SEQ ID NO: 93 - protein, 11 aa, synthetic construct
SASQGISNYLN

SEQ ID NO: 94 - protein, 6 aa, synthetic construct
QSVSND

SEQ ID NO: 95 - protein, 6 aa, synthetic construct
QDISNY

SEQ ID NO: 96 - protein, 6 aa, synthetic construct
QDINKY

SEQ ID NO: 97 - protein, 6 aa, synthetic construct
QEISGY

SEQ ID NO: 98 - protein, 6 aa, synthetic construct
QDVGIA

SEQ ID NO: 99 - protein, 6 aa, synthetic construct
ENIYSY

SEQ ID NO: 100 - protein, 7 aa, synthetic construct
YASNRYT

SEQ ID NO: 101 - protein, 7 aa, synthetic construct
YTSRLHS

SEQ ID NO: 102 - protein, 7 aa, synthetic construct
YTSTLQP

SEQ ID NO: 103 - protein, 7 aa, synthetic construct
AASTLDS

SEQ ID NO: 104 - protein, 7 aa, synthetic construct
WASTRHT

SEQ ID NO: 105 - protein, 7 aa, synthetic construct
NAKTLAE

SEQ ID NO: 106 - protein, 7 aa, synthetic construct
YTSSLHS

SEQ ID NO: 107-111 - skipped (5 entries)

SEQ ID NO: 112 - protein, 9 aa, synthetic construct
QQDYSSPLA

SEQ ID NO: 113 - protein, 8 aa, synthetic construct
QQGNTLYT

SEQ ID NO: 114 - protein, 8 aa, synthetic construct
LQYDNLYT

SEQ ID NO: 115 - protein, 9 aa, synthetic construct
LQYASYPYT

SEQ ID NO: 116 - protein, 9 aa, synthetic construct
QQYSSYPYT

SEQ ID NO: 117 - protein, 9 aa, synthetic construct
QHHYGSPPT

SEQ ID NO: 118 - protein, 9 aa, synthetic construct
QHHYGTPLT

SEQ ID NO: 119 - protein, 9 aa, synthetic construct
LQYASYPWT

SEQ ID NO: 120 - protein, 9 aa, synthetic construct
QQYSKLPFT

SEQ ID NO: 121 - protein, 18 aa, synthetic construct
GSTSGSGKPGSGEGSTKG

SEQ ID NO: 122 - DNA, 375 bp, synthetic construct
  source 1..375: mol_type = other DNA
caggttcagctggtgcagtctggagctgaggtgaagaagcctggggcctcagtgaaggtctcctgcaaggcttctggttacacctttaaagaatatggtatcagctgggtgcgacaggcccctggacaagggcttgagtggatgggatggatcagcgcttacagtggtcacacatactatgcacagaagctccagggcagagtcaccatgaccacagacacatccacgagcacagcctacatggagctgaggagcctgagatctgacgacacggcggtgtactactgcgccagagggcctcactacgacgactggagcggatttatcatatggttcgacccatggggacagggtacattggtcaccgtctcctca

SEQ ID NO: 123 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccttcaaggttcagtggcagtggatctgggacagatttcactctcaccatcagcagtctgcaacctgaagattttgcaacttactactgtcaacagagttacaggtttcctcctacctttggccaagggaccaaggttgagatcaaa

SEQ ID NO: 124 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 125 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 126 - protein, 15 aa, synthetic construct
GGGGSGGGGSGGGGS

SEQ ID NO: 127 - protein, 7 aa, synthetic construct
GYTFTNY

SEQ ID NO: 128 - protein, 7 aa, synthetic construct
GFTFSSY

SEQ ID NO: 129 - protein, 8 aa, synthetic construct
DFSITSDY

SEQ ID NO: 130 - protein, 7 aa, synthetic construct
GYTFTHY

SEQ ID NO: 131 - protein, 7 aa, synthetic construct
GYTFTDY

SEQ ID NO: 132 - protein, 7 aa, synthetic construct
GFTFSNY

SEQ ID NO: 133 - protein, 7 aa, synthetic construct
GYTFTDY

SEQ ID NO: 134 - protein, 7 aa, synthetic construct
GYTFTNY

SEQ ID NO: 135 - protein, 7 aa, synthetic construct
GYTFTSH

SEQ ID NO: 136 - protein, 7 aa, synthetic construct
GYTFTSY

SEQ ID NO: 137 - protein, 7 aa, synthetic construct
GYTFTSY

SEQ ID NO: 138 - protein, 7 aa, synthetic construct
GYTFTNY

SEQ ID NO: 139 - protein, 6 aa, synthetic construct
YPSDSE

SEQ ID NO: 140 - protein, 6 aa, synthetic construct
NSGGSY

SEQ ID NO: 141 - protein, 5 aa, synthetic construct
HYSGG

SEQ ID NO: 142 - protein, 6 aa, synthetic construct
YPSDGE

SEQ ID NO: 143 - protein, 6 aa, synthetic construct
TPNKGD

SEQ ID NO: 144 - protein, 6 aa, synthetic construct
SYSGAY

SEQ ID NO: 145 - protein, 6 aa, synthetic construct
STYSGK

SEQ ID NO: 146 - protein, 6 aa, synthetic construct
YPSDSE

SEQ ID NO: 147 - protein, 6 aa, synthetic construct
DPNSGG

SEQ ID NO: 148 - protein, 6 aa, synthetic construct
YPSDSE

SEQ ID NO: 149 - protein, 6 aa, synthetic construct
YPSDSE

SEQ ID NO: 150 - protein, 6 aa, synthetic construct
YPSDSE

SEQ ID NO: 151 - protein, 14 aa, synthetic construct
HLIYYDYDGYYFDT

SEQ ID NO: 152 - protein, 9 aa, synthetic construct
YGSRDAVDY

SEQ ID NO: 153 - protein, 15 aa, synthetic construct
GKNYSGSSLHWYFDV

SEQ ID NO: 154 - protein, 14 aa, synthetic construct
HVEYYDYDPYALDF

SEQ ID NO: 155 - protein, 9 aa, synthetic construct
GGLRDAMDY

SEQ ID NO: 156 - protein, 12 aa, synthetic construct
EGYEGYLGYFDF

SEQ ID NO: 157 - protein, 5 aa, synthetic construct
NYWMD

SEQ ID NO: 158 - protein, 5 aa, synthetic construct
SYVLS

SEQ ID NO: 159 - protein, 6 aa, synthetic construct
SDYTWH

SEQ ID NO: 160 - protein, 5 aa, synthetic construct
HYWMD

SEQ ID NO: 161 - protein, 5 aa, synthetic construct
DYYMN

SEQ ID NO: 162 - protein, 5 aa, synthetic construct
NYGMS

SEQ ID NO: 163 - protein, 5 aa, synthetic construct
DYTMH

SEQ ID NO: 164 - protein, 5 aa, synthetic construct
NYWMD

SEQ ID NO: 165 - protein, 5 aa, synthetic construct
SHWIH

SEQ ID NO: 166 - protein, 5 aa, synthetic construct
SYWMD

SEQ ID NO: 167 - protein, 5 aa, synthetic construct
SYWMD

SEQ ID NO: 168 - protein, 5 aa, synthetic construct
NYWMD

SEQ ID NO: 169 - protein, 17 aa, synthetic construct
HIYPSDSETHYIQKFKD

SEQ ID NO: 170 - protein, 17 aa, synthetic construct
TINSGGSYTYYPDSVKG

SEQ ID NO: 171 - protein, 16 aa, synthetic construct
YIHYSGGTNYNPSLRS

SEQ ID NO: 172 - protein, 17 aa, synthetic construct
HIYPSDGETHYNQKFRD

SEQ ID NO: 173 - protein, 17 aa, synthetic construct
DITPNKGDTNYNQKFKD

SEQ ID NO: 174 - protein, 17 aa, synthetic construct
TISYSGAYTYYPDSVKG

SEQ ID NO: 175 - protein, 17 aa, synthetic construct
LISTYSGKTNYNQKLKD

SEQ ID NO: 176 - protein, 17 aa, synthetic construct
NIYPSDSETHYNQKFRD

SEQ ID NO: 177 - protein, 17 aa, synthetic construct
RIDPNSGGTKYNEKFKS

SEQ ID NO: 178 - protein, 17 aa, synthetic construct
HIYPSDSETHYNQKFKD

SEQ ID NO: 179 - protein, 17 aa, synthetic construct
NIYPSDSETHYNQKFKD

SEQ ID NO: 180 - protein, 17 aa, synthetic construct
NIYPSDSETHYNQNFKD

SEQ ID NO: 181 - protein, 14 aa, synthetic construct
HLIYYDYDGYYFDT

SEQ ID NO: 182 - protein, 9 aa, synthetic construct
YGSRDAVDY

SEQ ID NO: 183 - protein, 15 aa, synthetic construct
GKNYSGSSLHWYFDV

SEQ ID NO: 184 - protein, 14 aa, synthetic construct
HVEYYDYDPYALDF

SEQ ID NO: 185 - protein, 9 aa, synthetic construct
GGLRDAMDY

SEQ ID NO: 186 - protein, 12 aa, synthetic construct
EGYEGYLGYFDF

SEQ ID NO: 187 - protein, 8 aa, synthetic construct
GYTFTNYW

SEQ ID NO: 188 - protein, 8 aa, synthetic construct
GFTFSSYV

SEQ ID NO: 189 - protein, 9 aa, synthetic construct
DFSITSDYT

SEQ ID NO: 190 - protein, 8 aa, synthetic construct
GYTFTHYW

SEQ ID NO: 191 - protein, 8 aa, synthetic construct
GYTFTDYY

SEQ ID NO: 192 - protein, 8 aa, synthetic construct
GFTFSNYG

SEQ ID NO: 193 - protein, 8 aa, synthetic construct
GYTFTDYT

SEQ ID NO: 194 - protein, 8 aa, synthetic construct
GYTFTNYW

SEQ ID NO: 195 - protein, 8 aa, synthetic construct
GYTFTSHW

SEQ ID NO: 196 - protein, 8 aa, synthetic construct
GYTFTSYW

SEQ ID NO: 197 - protein, 8 aa, synthetic construct
GYTFTSYW

SEQ ID NO: 198 - protein, 8 aa, synthetic construct
GYTFTNYW

SEQ ID NO: 199 - protein, 8 aa, synthetic construct
IYPSDSET

SEQ ID NO: 200 - protein, 8 aa, synthetic construct
INSGGSYT

SEQ ID NO: 201 - protein, 7 aa, synthetic construct
IHYSGGT

SEQ ID NO: 202 - protein, 8 aa, synthetic construct
IYPSDGET

SEQ ID NO: 203 - protein, 8 aa, synthetic construct
ITPNKGDT

SEQ ID NO: 204 - protein, 8 aa, synthetic construct
ISYSGAYT

SEQ ID NO: 205 - protein, 8 aa, synthetic construct
ISTYSGKT

SEQ ID NO: 206 - protein, 8 aa, synthetic construct
IYPSDSET

SEQ ID NO: 207 - protein, 8 aa, synthetic construct
IDPNSGGT

SEQ ID NO: 208 - protein, 8 aa, synthetic construct
IYPSDSET

SEQ ID NO: 209 - protein, 8 aa, synthetic construct
IYPSDSET

SEQ ID NO: 210 - protein, 8 aa, synthetic construct
IYPSDSET

SEQ ID NO: 211 - protein, 5 aa, synthetic construct
ARDYY

SEQ ID NO: 212 - protein, 16 aa, synthetic construct
ARHLIYYDYDGYYFDT

SEQ ID NO: 213 - protein, 11 aa, synthetic construct
LYYGSRDAVDY

SEQ ID NO: 214 - protein, 5 aa, synthetic construct
ARDYY

SEQ ID NO: 215 - protein, 17 aa, synthetic construct
ARGKNYSGSSLHWYFDV

SEQ ID NO: 216 - protein, 16 aa, synthetic construct
SRHVEYYDYDPYALDF

SEQ ID NO: 217 - protein, 11 aa, synthetic construct
ARGGLRDAMDY

SEQ ID NO: 218 - protein, 5 aa, synthetic construct
ARDYY

SEQ ID NO: 219 - protein, 14 aa, synthetic construct
AREGYEGYLGYFDF

SEQ ID NO: 220 - protein, 5 aa, synthetic construct
ARDYY

SEQ ID NO: 221 - protein, 5 aa, synthetic construct
ARDYY

SEQ ID NO: 222 - protein, 5 aa, synthetic construct
ARDYY

SEQ ID NO: 223 - protein, 11 aa, synthetic construct
RASKNISKYLA

SEQ ID NO: 224 - protein, 14 aa, synthetic construct
RSSTGAVTTSNYAN

SEQ ID NO: 225 - protein, 15 aa, synthetic construct
RASESVDSYGNSFMH

SEQ ID NO: 226 - protein, 10 aa, synthetic construct
STSSSVTYMH

SEQ ID NO: 227 - protein, 14 aa, synthetic construct
RSNTGAVTTSNYAN

SEQ ID NO: 228 - protein, 16 aa, synthetic construct
RSSQTLVHSNGNIYLH

SEQ ID NO: 229 - protein, 14 aa, synthetic construct
RSSTGAVTTSNYAN

SEQ ID NO: 230 - protein, 14 aa, synthetic construct
RSSTGAVTRSNYAN

SEQ ID NO: 231 - protein, 14 aa, synthetic construct
RSSTGAVTRSNYAN

SEQ ID NO: 232 - protein, 14 aa, synthetic construct
RSSTGAVTTSNYAN

SEQ ID NO: 233 - protein, 7 aa, synthetic construct
SGSTLQS

SEQ ID NO: 234 - protein, 7 aa, synthetic construct
GTNNRAL

SEQ ID NO: 235 - protein, 7 aa, synthetic construct
RASNLES

SEQ ID NO: 236 - protein, 7 aa, synthetic construct
DTSKLAS

SEQ ID NO: 237 - protein, 7 aa, synthetic construct
GTNNRVP

SEQ ID NO: 238 - protein, 7 aa, synthetic construct
KVSNRFS

SEQ ID NO: 239 - protein, 7 aa, synthetic construct
GTNNRAP

SEQ ID NO: 240 - protein, 7 aa, synthetic construct
GTNNRAL

SEQ ID NO: 241 - protein, 7 aa, synthetic construct
GTNNRAP

SEQ ID NO: 242 - protein, 7 aa, synthetic construct
GTNNRAP

SEQ ID NO: 243 - protein, 9 aa, synthetic construct
QQHYEYPYT

SEQ ID NO: 244 - protein, 9 aa, synthetic construct
ALWYSNHLV

SEQ ID NO: 245 - protein, 9 aa, synthetic construct
LQSNEDPRT

SEQ ID NO: 246 - protein, 9 aa, synthetic construct
QQWSSSPYT

SEQ ID NO: 247 - protein, 9 aa, synthetic construct
ALWYSNHWV

SEQ ID NO: 248 - protein, 9 aa, synthetic construct
SQTTHVPPT

SEQ ID NO: 249 - protein, 9 aa, synthetic construct
ALWYSNHLV

SEQ ID NO: 250 - protein, 9 aa, synthetic construct
ALWYSNHLV

SEQ ID NO: 251 - protein, 9 aa, synthetic construct
ALWYSNHLV

SEQ ID NO: 252 - protein, 9 aa, synthetic construct
ALWYSSQLV

SEQ ID NO: 253 - protein, 11 aa, synthetic construct
RASKNISKYLA

SEQ ID NO: 254 - protein, 14 aa, synthetic construct
RSSTGAVTTSNYAN

SEQ ID NO: 255 - protein, 15 aa, synthetic construct
RASESVDSYGNSFMH

SEQ ID NO: 256 - protein, 10 aa, synthetic construct
STSSSVTYMH

SEQ ID NO: 257 - protein, 14 aa, synthetic construct
RSNTGAVTTSNYAN

SEQ ID NO: 258 - protein, 16 aa, synthetic construct
RSSQTLVHSNGNIYLH

SEQ ID NO: 259 - protein, 14 aa, synthetic construct
RSSTGAVTTSNYAN

SEQ ID NO: 260 - protein, 14 aa, synthetic construct
RSSTGAVTRSNYAN

SEQ ID NO: 261 - protein, 14 aa, synthetic construct
RSSTGAVTRSNYAN

SEQ ID NO: 262 - protein, 14 aa, synthetic construct
RSSTGAVTTSNYAN

SEQ ID NO: 263 - protein, 7 aa, synthetic construct
SGSTLQS

SEQ ID NO: 264 - protein, 7 aa, synthetic construct
GTNNRAL

SEQ ID NO: 265 - protein, 7 aa, synthetic construct
RASNLES

SEQ ID NO: 266 - protein, 7 aa, synthetic construct
DTSKLAS

SEQ ID NO: 267 - protein, 7 aa, synthetic construct
GTNNRVP

SEQ ID NO: 268 - protein, 7 aa, synthetic construct
KVSNRFS

SEQ ID NO: 269 - protein, 7 aa, synthetic construct
GTNNRAP

SEQ ID NO: 270 - protein, 7 aa, synthetic construct
GTNNRAL

SEQ ID NO: 271 - protein, 7 aa, synthetic construct
GTNNRAP

SEQ ID NO: 272 - protein, 7 aa, synthetic construct
GTNNRAP

SEQ ID NO: 273 - protein, 9 aa, synthetic construct
QQHYEYPYT

SEQ ID NO: 274 - protein, 9 aa, synthetic construct
ALWYSNHLV

SEQ ID NO: 275 - protein, 9 aa, synthetic construct
LQSNEDPRT

SEQ ID NO: 276 - protein, 9 aa, synthetic construct
QQWSSSPYT

SEQ ID NO: 277 - protein, 9 aa, synthetic construct
ALWYSNHWV

SEQ ID NO: 278 - protein, 9 aa, synthetic construct
SQTTHVPPT

SEQ ID NO: 279 - protein, 9 aa, synthetic construct
ALWYSNHLV

SEQ ID NO: 280 - protein, 9 aa, synthetic construct
ALWYSNHLV

SEQ ID NO: 281 - protein, 9 aa, synthetic construct
ALWYSNHLV

SEQ ID NO: 282 - protein, 9 aa, synthetic construct
ALWYSSQLV

SEQ ID NO: 283 - protein, 6 aa, synthetic construct
KNISKY

SEQ ID NO: 284 - protein, 9 aa, synthetic construct
TGAVTTSNY

SEQ ID NO: 285 - protein, 10 aa, synthetic construct
ESVDSYGNSF

SEQ ID NO: 286 - protein, 5 aa, synthetic construct
SSVTY

SEQ ID NO: 287 - protein, 9 aa, synthetic construct
TGAVTTSNY

SEQ ID NO: 288 - protein, 11 aa, synthetic construct
QTLVHSNGNIY

SEQ ID NO: 289 - protein, 9 aa, synthetic construct
TGAVTTSNY

SEQ ID NO: 290 - protein, 9 aa, synthetic construct
TGAVTRSNY

SEQ ID NO: 291 - protein, 9 aa, synthetic construct
TGAVTRSNY

SEQ ID NO: 292 - protein, 9 aa, synthetic construct
TGAVTTSNY

SEQ ID NO: 293 - protein, 9 aa, synthetic construct
QQHYEYPYT

SEQ ID NO: 294 - protein, 9 aa, synthetic construct
ALWYSNHLV

SEQ ID NO: 295 - protein, 9 aa, synthetic construct
LQSNEDPRT

SEQ ID NO: 296 - protein, 9 aa, synthetic construct
QQWSSSPYT

SEQ ID NO: 297 - protein, 9 aa, synthetic construct
ALWYSNHWV

SEQ ID NO: 298 - protein, 9 aa, synthetic construct
SQTTHVPPT

SEQ ID NO: 299 - protein, 9 aa, synthetic construct
ALWYSNHLV

SEQ ID NO: 300 - protein, 9 aa, synthetic construct
ALWYSNHLV

SEQ ID NO: 301 - protein, 9 aa, synthetic construct
ALWYSNHLV

SEQ ID NO: 302 - protein, 9 aa, synthetic construct
ALWYSSQLV

SEQ ID NO: 303 - protein, 112 aa, synthetic construct
QVQLQQPGAELVRPGSSVKLSCKASGYTFTNYWMDWVKQRPGQGLEWIGHIYPSDSETHYIQKFKDKATLTVDKSSNTAYMQLSSLTSEDSAVYYCARDYYWGQGTTLTVSS

SEQ ID NO: 304 - protein, 123 aa, synthetic construct
EVQLVESGGGLVKPGGSLKLSCAASGFTFSSYVLSWVRQIPEKRLEWVATINSGGSYTYYPDSVKGRFTISRDNAKNTLYLQMSSLRSEDTAIYYCARHLIYYDYDGYYFDTWGQGTSLTVSS

SEQ ID NO: 305 - protein, 118 aa, synthetic construct
DVQLQESGPDLVKPSQSLSLTCTVTDFSITSDYTWHWVRQFPGNKLEWMAYIHYSGGTNYNPSLRSRISITRDTSKNQFFLHMNSVTTEDTATYYCLYYGSRDAVDYWGQGTSVTVSS

SEQ ID NO: 306 - protein, 112 aa, synthetic construct
QVQLQQPGAELVRPGSSVKLSCKASGYTFTHYWMDWVKQRPGQGLEWIGHIYPSDGETHYNQKFRDKATLTVDKSSSTAYVQLSSLTSEDSAVYYCARDYYWGQGTTLTVSS

SEQ ID NO: 307 - protein, 124 aa, synthetic construct
EVQLQQSGPELVKPGASVKISCKASGYTFTDYYMNWVKQSHGKSLEWIGDITPNKGDTNYNQKFKDRATLTVDKSSNTAYMELRSLTSEDSSVYYCARGKNYSGSSLHWYFDVWGTGTTVTVSS

SEQ ID NO: 308 - protein, 123 aa, synthetic construct
EVQLVESGGDLVKPGGSLKLSCAASGFTFSNYGMSWVRQTPDKRLEWVATISYSGAYTYYPDSVKGRFTISRDNAKNTLYLQMGSLKSEDTAMFFCSRHVEYYDYDPYALDFWGQATSVTVSS

SEQ ID NO: 309 - protein, 118 aa, synthetic construct
QVQLQQSGPELVRPGVSVKISCKVSGYTFTDYTMHWVKQSHAKSLEWIGLISTYSGKTNYNQKLKDKATMTVDKSSSTAYMELARLTSEDSAIFYCARGGLRDAMDYWGQGTSVTVSS

SEQ ID NO: 310 - protein, 112 aa, synthetic construct
QVQLQQPGAELVRPGSSVKLSCKASGYTFTNYWMDWVKQRPGQGLEWIGNIYPSDSETHYNQKFRDKATLTVDKPSSTAYMQLSSLTSEDSAVYYCARDYYWGQGTTLTVSS

SEQ ID NO: 311 - protein, 121 aa, synthetic construct
QVQLQQPGAEFVKPGASVKLSCEASGYTFTSHWIHWVKQRPGRGLDWIGRIDPNSGGTKYNEKFKSKATLTVDKPSSTAYMQLSSLTSEDPAVHYCAREGYEGYLGYFDFWGQGTTLTVSS

SEQ ID NO: 312 - protein, 112 aa, synthetic construct
QVQLQQPGAELVRPGSSVKLSCKASGYTFTSYWMDWMKQRPGQGLEWIGHIYPSDSETHYNQKFKDRATLTVDKSSSAAFMQLSSLTSEDSAVYYCARDYYWGQGTTLTVSS

SEQ ID NO: 313 - protein, 112 aa, synthetic construct
QVQLQQPGAELVRPGSSVRLSCKASGYTFTSYWMDWVKQRPGQGLEWIGNIYPSDSETHYNQKFKDKATLTVDKSSSTAYMQLSSLTSEDSAVYYCARDYYWGQGTTLTVSS

SEQ ID NO: 314 - protein, 112 aa, synthetic construct
QVQLQQPGAELVRPGSSVKLSCKASGYTFTNYWMDWVKQRPGQGLEWIGNIYPSDSETHYNQNFKDKATLTVDKSSSTAYMQLSSLTSEDSAVYYCARDYYWGQGTTLTVSS

SEQ ID NO: 315 - protein, 111 aa, synthetic construct
DVQITQSPSYLAASPGETITINCRASKNISKYLAWYQEKPGKTNKLLIYSGSTLQSGNPSRFSASGSGTDFTLTISSLEPEDFAMYYCQQHYEYPYTFGGGTKLEIKLEIK

SEQ ID NO: 316 - protein, 112 aa, synthetic construct
QAVVTQESALTTSPGETVTLTCRSSTGAVTTSNYANWVQEKPDHLFTGLIGGTNNRALGVPARFSGSLIGDKAALTITGAQIEDEAIYFCALWYSNHLVFGGGTKLTVLQPK

SEQ ID NO: 317 - protein, 111 aa, synthetic construct
DIVLTQSPASLAVSLGQGATISCRASESVDSYGNSFMHWYQQKPGQPPKLLIYRASNLESGIPARFSGSGSGTDFTLTINPVEADDVATYYCLQSNEDPRTFGGGTKLEIK

SEQ ID NO: 318 - protein, 106 aa, synthetic construct
QIVLTQSPAIMSASPGEKVTMTCSTSSSVTYMHWYQQKSGTSPKRWIYDTSKLASGVPARFSGSGSGTSYSLTISSMEAEDAATYYCQQWSSSPYTFGGGTKLEIK

SEQ ID NO: 319 - protein, 112 aa, synthetic construct
QAVVTQESALTTSPGETVTLTCRSNTGAVTTSNYANWVQERPDHLFTGLIGGTNNRVPGVPARFSGSLIGDKAALTITGAQTEDEAIYFCALWYSNHWVFGGGTKLTVLQPK

SEQ ID NO: 320 - protein, 112 aa, synthetic construct
DVVMTQTPLSLPVSLGDQASISCRSSQTLVHSNGNIYLHWYLQKPGQSPKLLIYKVSNRFSGVPDRFSGSGSGTDFTLKISRVETEDLGIYFCSQTTHVPPTFGGGTKLEIK

SEQ ID NO: 321 - protein, 112 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVLQPK

SEQ ID NO: 322 - protein, 112 aa, synthetic construct
QAVVTQESALTTSPGETVTLTCRSSTGAVTRSNYANWVQEKPDHLFTGLIGGTNNRALGVPARFSGSLIGDKAALTITGAQTEDEAIYFCALWYSNHLVFGGGTKLTVLQPK

SEQ ID NO: 323 - protein, 112 aa, synthetic construct
QAVVTQESALTTSPGETVTLTCRSSTGAVTRSNYANWVQEKPDHLFTGLIGGTNNRAPGVPARFSGSLIGDKAALTITGAQTEDEAIYFCALWYSNHLVFGGGTKLTVLQPK

SEQ ID NO: 324 - protein, 112 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSSQLVFGGGTKLTVLQPK

SEQ ID NO: 325 - protein, 685 aa, Gibbon ape leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 326 - DNA, 369 bp, synthetic construct
  source 1..369: mol_type = other DNA
gaagtgcagctggtggagtctgggggaggcttagtgaagcctggagggtccctgaaactctcctgtgcagcctctggattcactttcagtagttatgtcctgtcttgggttcgccagattccggagaagaggctggagtgggtcgcaaccattaatagtggtggtagttacacttactatccagacagtgtgaagggtcgattcaccatctccagagacaatgccaagaacaccctgtacctgcaaatgagcagtctgaggtctgaggacacggccatatattactgtgcacgacatctcatctactatgattacgacggttactactttgacacctggggccaaggcacctctctcacagtctcctca

SEQ ID NO: 327 - DNA, 354 bp, synthetic construct
  source 1..354: mol_type = other DNA
gatgtgcagcttcaggagtcaggacctgacctagtgaaaccttctcagtcgctttcactcacctgcactgtcactgacttctccatcaccagtgattatacctggcactgggtccggcagtttccaggaaacaaactggaatggatggcctacatacactacagtggtggcactaactacaatccatctctcagaagtcgaatctctatcactcgagacacatccaagaaccagttcttcctgcatatgaattctgtgactactgaggacacagccacatattactgtctttactacggtagtagagatgctgtggactactggggtcaaggaacctcagtcaccgtctcctca

SEQ ID NO: 328 - DNA, 336 bp, synthetic construct
  source 1..336: mol_type = other DNA
caggtccaactacagcaacctggggctgaactggtgaggcctgggtcttcagtgaaactgtcctgcaaggcttctggctacaccttcacccactactggatggattgggtgaagcagaggcctggacaaggccttgaatggattggtcacatttacccttctgatggtgaaactcactacaatcaaaagttcagggacaaggccacattgactgtagacaaatcctccagcacagcctacgtgcagctcagcagcctgacatctgaggactctgcggtctattattgtgcaagagactattactggggccaaggcaccactctcacagtctcctca

SEQ ID NO: 329 - DNA, 372 bp, synthetic construct
  source 1..372: mol_type = other DNA
gaagtccagctgcarcagtctggacctgagctggtgaagcctggggcttctgtgaagatatcctgtaaggcttctggatacacgttcactgactactacatgaactgggtgaagcagagccatggaaagagccttgagtggattggagatattactcctaacaagggtgatactaactacaaccagaagttcaaggacagggccacattgactgtcgacaagtcctccaacacagcctacatggagctccgcagcctgacatctgaggactcttcagtctattactgtgcaagagggaaaaattactccggtagtagccttcactggtacttcgatgtctggggcacagggaccacggtcaccgtctcctca

SEQ ID NO: 330 - DNA, 369 bp, synthetic construct
  source 1..369: mol_type = other DNA
gaggtgcagctggtggagtctgggggagacttagtgaagcctggagggtccctgaaactctcctgtgcagcctctggattcactttcagtaactatggcatgtcttgggttcgccagactccagacaagagactggagtgggtcgcaaccattagttatagtggtgcttacacctactatccagacagtgtgaaggggcgattcaccatctccagagacaatgccaagaacaccctgtacctgcaaatgggcagtctgaagtctgaggacacagccatgtttttctgttcaagacatgttgagtactatgattacgacccttatgctttggacttctggggtcaagcaacctcagtcaccgtctcctca

SEQ ID NO: 331 - DNA, 354 bp, synthetic construct
  source 1..354: mol_type = other DNA
caggtccagctgcagcagtctgggcctgagctggtgaggcctggggtctcagtgaagatttcctgcaaggtttccggctacacattcactgattatactatgcattgggtgaagcagagtcatgcaaagagtctagagtggattggacttattagcacttactctggtaaaacaaactacaaccagaaacttaaggacaaggccacaatgactgtggacaaatcctccagcacagcctatatggaacttgccagattgacatctgaggattctgccatcttttactgtgcaagagggggattaagggatgctatggactactggggtcaaggaacctcagtcaccgtctcctca

SEQ ID NO: 332 - DNA, 336 bp, synthetic construct
  source 1..336: mol_type = other DNA
caggtccaactgcagcagcctggggctgagctggtgaggcctgggtcttcagtgaaactgtcctgcaaggcttctggctacaccttcaccaactactggatggattgggtgaaacagaggcctggacaaggccttgaatggattggtaacatttacccttctgatagtgaaactcactacaatcaaaagttcagggacaaggccacattgactgtagacaaaccctccagcacagcctacatgcagctcagcagcctgacatctgaggactctgcggtctattattgtgcaagagactattactggggccaaggcaccactctcacagtctcctca

SEQ ID NO: 333 - DNA, 363 bp, synthetic construct
  source 1..363: mol_type = other DNA
caggtccaactgcagcagcctggggctgagtttgtgaagcctggggcttcagtgaagctgtcctgtgaggcttctggctacaccttcaccagccactggatacactgggtgaagcagaggcctggacgaggccttgactggattggaaggattgatcctaatagtggtggtactaagtacaatgagaagttcaagagcaaggccacactgactgtagacaaaccctccagcacagcctacatgcagctcagcagcctgacatctgaggaccctgcggtccattactgtgcaagagagggctatgagggttacttgggttactttgacttctggggccaaggcaccactctcacagtttcctca

SEQ ID NO: 334 - DNA, 336 bp, synthetic construct
  source 1..336: mol_type = other DNA
caggtccaactgcagcagcctggggctgagctggtgaggcctgggtcttcagtgaaactatcctgcaaggcttctggctacaccttcaccagctactggatggattggatgaaacagaggcctggacaaggccttgaatggattggtcacatttacccttctgatagtgaaactcactacaatcaaaagttcaaggacagggccacattgactgtagacaaatcctccagcgcagccttcatgcaactcagcagcctgacatctgaggactctgcggtctattactgtgcaagagattactactggggccaaggcaccactctcacagtctcctca

SEQ ID NO: 335 - DNA, 336 bp, synthetic construct
  source 1..336: mol_type = other DNA
caggtccaactacagcaacctggggctgagctggtgaggcctgggtcttcagtgagactgtcctgcaaggcttctggctacaccttcaccagctactggatggattgggtgaagcagaggcctggacagggccttgaatggattggtaacatttacccttctgatagtgaaactcactacaatcaaaagttcaaggacaaggccacattgactgtagacaaatcctccagcacagcctacatgcagctcagcagcctgacatctgaggactctgcggtctattactgtgcaagagactactactggggccaaggcaccactctcacagtctcctca

SEQ ID NO: 336 - DNA, 336 bp, synthetic construct
  source 1..336: mol_type = other DNA
caggtccaactacagcaacctggggctgagctggtgaggcctgggtcttcagtgaaactgtcctgcaaggcttctggctacaccttcaccaactactggatggattgggtgaagcagaggcctggacagggccttgaatggattggtaacatttacccttctgatagtgaaactcactacaatcaaaacttcaaggacaaggccacattgactgtagacaaatcctccagcacagcctacatgcagctcagcagcctgacatctgaggactctgcggtctattactgtgcaagagactactactggggccaaggcaccactctcacagtctcctca

SEQ ID NO: 337 - DNA, 321 bp, synthetic construct
  source 1..321: mol_type = other DNA
gatgtccagatactccagtctccatcttatcttgctgcatctcctggagaaaccattactattaattgcagggcaagtaagaacattagcaaatatttagcctggtatcaagagaaacctgggaaaactaataaacttcttatctactctggatccactttgcaatctggaaatccatcaaggttcagtgccagtggatctggtacagatttcactctcaccatcagtagcctggagcctgaagattttgcaatgtattactgtcaacagcattatgaatacccgtacacgttcggaggggggaccaagctggaaataaaa

SEQ ID NO: 338 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctctaggtgttcctgccagattctcaggctccctgattggagacaaggctgccctcaccatcacaggggcacagattgaggatgaggcaatatatttctgtgctctatggtacagcaaccatttggtgttcggtggaggaaccaaactgactgtccta

SEQ ID NO: 339 - DNA, 333 bp, synthetic construct
  source 1..333: mol_type = other DNA
gacattgtgctgacccaatctccagcttctttggctgtgtctctagggcagggggccaccatatcctgcagagccagtgaaagtgttgatagttatggcaatagttttatgcactggtaccagcagaaaccaggacagccacccaaactcctcatctatcgtgcatccaacctagaatctgggatccctgccaggttcagtggcagtgggtctgggacagacttcaccctcaccattaatcctgtggaggctgatgatgttgcaacctattactgtctgcaaagtaatgaggatcctcggacgttcggtggaggcaccaagctggaaatcaaa

SEQ ID NO: 340 - DNA, 318 bp, synthetic construct
  source 1..318: mol_type = other DNA
caaattgttctcacccagtctccagcaatcatgtctgcatctccaggggagaaggtcaccatgacctgcagtaccagctcaagtgtaacttatatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtagcccatacacgttcggaggggggaccaagctggaaataaaa

SEQ ID NO: 341 - DNA, 327 bp, synthetic construct
  source 1..327: mol_type = other DNA
caggctgttgtaactcaggaatctgcactcaccacatcacctggtgaaacagtcacactcacttgtcgctcaaatactggggctgttacaaccagtaactatgccaactgggtccaagaaagaccagatcatttattcactggtctaataggtggtaccaacaaccgagttccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 342 - DNA, 336 bp, synthetic construct
  source 1..336: mol_type = other DNA
gatgttgtgatgacccaaactccactctccctgcctgtcagtcttggagatcaagcctccatctcttgcagatctagtcagacccttgttcacagtaatggaaatatctatttacattggtacctgcagaagccaggccagtctccaaagctcctgatctacaaagtttccaaccgattttctggagtcccagacaggttcagtggcagtggatcagggacagatttcacactcaagatcagcagagtggagactgaggatctgggaatttatttctgctctcaaaccacacatgttcctccgacgttcggtggaggcaccaaactggaaatcaaa

SEQ ID NO: 343 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccaaactgactgtccta

SEQ ID NO: 344 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaaggagtaactatgccaactgggtccaagaaaaaccagatcatttattcactggtctaataggtggtaccaacaatcgagctctaggtgttcctgccagattctcaggctccctgattggagacaaggctgccctcaccatcacaggggcacagactgaggatgaggcaatatatttctgtgctctatggtacagcaaccatttggtgttcggtgggggaaccaaactgactgtccta

SEQ ID NO: 345 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaaggagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccaaactgactgtccta

SEQ ID NO: 346 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagtagccaattggtgttcggtggaggaaccaaactgactgtccta

SEQ ID NO: 347 - DNA, 336 bp, synthetic construct
  source 1..336: mol_type = other DNA
caggtccaactgcagcagcctggggctgagctggtgaggcctgggtcttcagtgaagctgtcctgcaaggcttctggctacaccttcaccaactactggatggattgggtgaagcagaggcctggacaaggccttgaatggattggtcacatttacccttctgatagtgaaactcactacattcaaaagttcaaggacaaggccacattgactgtagacaaatcctccaacacagcctacatgcagctcagcagcctgacatctgaggactctgcggtctattactgtgcaagagattactactggggccaaggcaccactctcacagtctcctca

SEQ ID NO: 348 - protein, 6 aa, synthetic construct
HHHHHH

(19) United States
(12) Patent Application Publication
(10) Pub. No.: US 2024/0343820 A1
(43) Pub. Date: Oct. 17, 2024

(54) ANTIGEN BINDING MOLECULES AND METHODS OF USE

(71) Applicant: Kite Pharma, Inc., Foster City, CA (US)

(72) Inventors: Michael E. Coon, Foster City, CA (US); Christa Cortesio, Orinda, CA (US); Stuart A. Sievers, Van Nuys, CA (US)

(21) Appl. No.: 18/597,752

(22) Filed: Mar. 6, 2024

Related U.S. Application Data
(60) Provisional application No. 63/620,111, filed on Jan. 11, 2024.
(60) Provisional application No. 63/489,373, filed on Mar. 9, 2023.

(51) Int. Cl. C07K 16/28; A61K 49/00; A61K 49/16; A61K 51/10; C07K 14/725

(52) U.S. Cl. CPC C07K 16/2887; A61K 49/0058; A61K 49/16; A61K 51/1027; C07K 14/7051

(57) ABSTRACT

The present disclosure describes antigen binding molecules, including antibodies, that specifically bind to the anti-CD20 scFv-14 or Gibbon ape leukemia virus gp70 protein, as well as molecules comprising the described sequences and cells presenting such molecules. The antigen binding molecules may be used in research, diagnostic, clinical, and other applications.

### CROSS REFERENCE TO RELATED APPLICATIONS

This application claims priority to U.S. Provisional Patent Application No. 63/489,373 filed on Mar. 9, 2023, and U.S. Provisional Patent Application No. 63/620,111, filed on Jan. 11, 2024, each of which is hereby incorporated in its entirety.

### SEQUENCE LISTING

The instant application contains a Sequence Listing which has been submitted electronically in .XML format and is hereby incorporated by reference in its entirety. Said .XML copy, created on Feb. 8, 2024, is named K-1137-WO-PCT_SL.xml and is 312,108 bytes in size.

### FIELD OF THE INVENTION

This disclosure relates to antigen binding molecules, such as antibodies, which specifically bind to targets, including an anti-CD20 scFv-14 binding domain or gibbon ape leukemia virus (GALV) protein gp70, as well as molecules comprising these sequences and cells presenting such molecules, polynucleotides encoding such antigen binding molecules, as well as humanized forms of the antigen binding molecules and methods of using the antigen binding molecules are also disclosed.

### BACKGROUND

Antigen binding molecules, including antibodies, and fragments such as Fabs, F(ab′)₂, scFvs, etc, are used in immunotherapy and solid phase-based applications such as biosensors, affinity chromatography, and immunoassays. These antibodies and other antigen binding molecules gain their utility by virtue of their ability to specifically bind their targets.

Anti-idiotypic antibodies are a subset of antibodies, and are antibodies raised against immunizing antibodies. These anti-idiotypic antibodies demonstrated specific binding against the idiotopes (unique antigenic determinants on the surface of the antibodies) of the immunizing antibodies. Anti-idiotypic antibodies can be generally classified into three distinct groups: (1) antibodies are those that recognize idiotopes distinct from the antigen-binding site (ABS) on immunizing antibodies; (2) antibodies that recognize epitopes within the ABS and mimic the structure, and forming the so-called “internal image,” of the nominal antigen; and (3) antibodies that recognize epitopes within the ABS without the structural resemblance of the nominal antigen (see, e.g., Pan et al., (1995) FASEB J 9:43-49).

There is a further need for the detection and quantification of viral particles. In particular, viral envelope proteins such as gibbon ape leukemia virus (GALV) protein gp70 provide excellent targets for viral detection. Antigen binding molecules specific for GALV gp70 would have many uses, for example in assays, such a flow based viral detection methods.

Disclosed herein are antigen binding molecules, including antibodies, that specifically bind to the anti-CD20 scFv-14 or GALV protein gp70, as well as molecules comprising these sequences and cells presenting such molecules. Humanized forms of the disclosed antigen binding molecules also form as aspect of the disclosure. Applications and uses of these antigen binding molecules are also disclosed.

### SUMMARY

In various aspects, an isolated antigen binding molecule that binds to anti-CD20 is disclosed. In various other aspects, an isolated antigen binding molecule that binds to gibbon ape leukemia virus (GALV) protein gp70 is disclosed. In various embodiments, a heavy chain variable (VH) sequence has at least about 80% sequence identity to a sequence selected from the group consisting of SEQ ID NOs: 1-10. In various embodiments, a light chain variable (VL) sequence has at least about 80% sequence identity to a sequence selected from the group consisting of SEQ ID NOs: 11-20. In various embodiments, a linker connects the VH to the VL.

In various aspects, an isolated antigen binding molecule is disclosed, which comprises a VH amino acid sequence that is at least about 85%, at least about 90%, at least about 95%, at least about 96%, at least about 97%, at least about 98%, at least about 99%, or about 100% identical to a VH of an antigen binding molecule described herein.

In various aspects, an isolated antigen binding molecule is disclosed, which comprises a VL amino acid sequence that is at least about 85%, at least about 90%, at least about 95%, at least about 96%, at least about 97%, at least about 98%, at least about 99%, or about 100% identical to a VL of an antigen binding molecule described herein.

In various embodiments, the isolated antigen binding molecule comprises a heavy chain CDR1 selected from the group consisting of SEQ ID NOs: 21-41. In various embodiments, the isolated antigen binding molecule comprises a heavy chain CDR2 selected from the group consisting of SEQ ID NOs: 42-65. In various embodiments, the isolated antigen binding molecule comprises a heavy chain CD3 selected from the group consisting of SEQ ID NOs: 66-85. In various embodiments, the isolated antigen binding molecule comprises a light chain CDR1 selected from the group consisting of SEQ ID NOs: 86-99. In various embodiments, the isolated antigen binding molecule comprises a light chain CDR2 selected from the group consisting of SEQ ID NOs: 100-111. In various embodiments, the antigen binding molecule comprises a light chain CDR3 selected from the group consisting of SEQ ID NOs: 112-120.

In various embodiments, the linker comprises an amino acid sequence. In various embodiments, the amino acid sequence of the linker comprises SEQ ID NO: 121. In various embodiments, the amino acid sequence of the linker comprises SEQ ID NO: 126. In various embodiments, an isolated antigen binding molecule, comprises a linker amino acid sequence that is at least about 70%, at least about 75%, at least about 80%, at least about 85%, at least about 90%, at least about 95%, at least about 96%, at least about 97%, at least about 98%, at least about 99%, or about 100% identical to a VL of an antigen binding molecule described herein.

In various embodiments, the isolated antigen binding molecule comprises a detectable label selected from the group consisting of a fluorescent label, a photochromic compound, a proteinaceous fluorescent label, a magnetic label, a radiolabel, and a hapten.

In various embodiments, the isolated antigen binding molecule comprises a heavy chain CDR1 sequence selected from the group consisting of SEQ ID NOs: 25, 32, and 39. In various embodiments, the isolated antigen binding molecule comprises a heavy chain CDR2 sequence selected from the group consisting of SEQ ID NOs: 46, 54, and 62. In various embodiments, the isolated antigen binding molecule comprises a heavy chain CDR3 sequence selected from the group consisting of SEQ ID NOs: 70 and 80. In various embodiments, the isolated antigen binding molecule comprises a light chain CDR1 sequence selected from the group consisting of SEQ ID NOs: 90 and 98. In various embodiments, the isolated antigen binding molecule comprises a light chain CDR2 sequence selected from the group consisting of SEQ ID NOs: 104 and 110. In various embodiments, the isolated antigen binding molecule comprises a light chain CDR3 sequence comprising SEQ ID NO: 116.

In certain aspects, the antigen binding system, antibody, or antigen binding fragment thereof comprises a GALV gp70 binding motif, wherein the GALV gp70 binding motif comprises sequences of three heavy chain complementarity determining regions (HCDRs) of any one of the heavy chain variable region (HCVR) selected from the group consisting of SEQ ID NOs: 303-314, and sequences of three light chain CDRs (LCDRs) of the light chain variable region (LCVR) selected from the group consisting of SEQ ID NOs:315-324.

In certain aspects, the GALV gp70 antigen binding system, antibody, or antigen binding fragment thereof comprises a first domain comprising three heavy chain complementarity determining regions (HCDR1, HCDR2 and HCDR3) and a second domain comprising three light chain complementarity determining regions (LCDR1, LCDR2 and LCDR3), wherein
(i) the HCDR1 has a sequence according to any one of SEQ ID NOs: 127-138, 157-168, and 187-198;
(ii) the HCDR2 has a sequence according to any one of SEQ ID NOs:139-150, 169-180, and 199-210;
(iii) the HCDR3 has a sequence according to any one of SEQ ID NOs:151-156, 181-186, 211-222 and DYY;
(iv) the LCDR1 has a sequence according to any one of SEQ ID NOs: 223-232, 253-262, and 283-292;
(v) the LCDR2 has a sequence according to any one of SEQ ID NOs: 233-242, 263-272, SGS, GTN, RAS, DTS, and KVS; and
(vi) the LCDR3 has a sequence according to any one of SEQ ID Nos: 243-252, 273-282, and 293-302.

In certain aspects, the GALV gp70 antigen binding system, antibody, or antigen binding fragment thereof comprise HCDRs which comprise:
(i) a HCDR1 according to any of SEQ ID Nos: 127, 157, and 187; a HCDR2 according to any of SEQ ID Nos: 139, 169, and 199; a HCDR3 according to SEQ ID NO: 211 or DYY;
(ii) a HCDR1 according to any of SEQ ID Nos: 128, 158 and 188; a HCDR2 according to any of SEQ ID Nos: 140, 170, and 200; a HCDR3 according to any one of SEQ ID Nos: 151, 181, and 212;
(iii) a HCDR1 according to any of SEQ ID Nos: 129, 159, and 189; a HCDR2 according to any of SEQ ID Nos: 141, 171, and 201; a HCDR3 according to any one of SEQ ID Nos: 152, 182, and 213;
(iv) a HCDR1 according to any of SEQ ID Nos: 130, 160, and 190; a HCDR2 according to any of SEQ ID Nos: 142, 172, and 202; a HCDR3 according to SEQ ID NO: 214 or DYY;
(v) a HCDR1 according to any of SEQ ID Nos: 131, 161, and 191; a HCDR2 according to any of SEQ ID Nos: 143, 173, and 203; a HCDR3 according to any one of SEQ ID Nos: 153, 183, and 215;
(vi) a HCDR1 according to any of SEQ ID Nos: 132, 162 and 192; a HCDR2 according to any of SEQ ID Nos: 144, 174 and 204; a HCDR3 according to any one of SEQ ID Nos: 154, 184, and 216;
(vii) a HCDR1 according to any of SEQ ID Nos: 133, 163, and 193; a HCDR2 according to any of SEQ ID Nos: 145, 175, and 205; a HCDR3 according to any one of SEQ ID Nos: 155, 185, and 217;
(viii) a HCDR1 according to any of SEQ ID Nos: 134, 164, and 194; a HCDR2 according to any of SEQ ID Nos: 146, 176, and 206; a HCDR3 according to SEQ ID NO: 218 or DYY;
(ix) a HCDR1 according to any of SEQ ID Nos: 135, 165, and 195; a HCDR2 according to any of SEQ ID Nos: 147, 177, and 207; a HCDR3 according to any one of SEQ ID Nos: 156, 186 and 219;
(x) a HCDR1 according to any one of SEQ ID Nos: 136, 166, and 196; a HCDR2 according to any of SEQ ID Nos: 148, 178, and 208; a HCDR3 according to SEQ ID NO: 220 or DYY;
(xi) a HCDR1 according to any one of SEQ ID Nos: 137, 167, and 197; a HCDR2 according to any of SEQ ID Nos: 149, 179, and 209; a HCDR3 according to SEQ ID NO: 221 or DYY; or
(xii) a HCDR1 according to any one of SEQ ID Nos: 138, 168, and 198; a HCDR2 according to any of SEQ ID Nos: 150, 180, and 210; a HCDR3 according to SEQ ID NO: 222 or DYY and
   and LCDRs which comprise:
(i) a LCDR1 according to any of SEQ ID Nos: 232, 262, and 292; a LCDR2 according to any of SEQ ID Nos: 242, 272 and GTN; a LCDR3 according to any one of SEQ ID Nos: 252, 282, and 302;
(ii) a LCDR1 according to any of SEQ ID Nos: 228, 258, and 288; a LCDR2 according to any of SEQ ID Nos: 238, 268, and KVS; a LCDR3 according to any one of SEQ ID Nos: 248, 278, and 298;
(iii) a LCDR1 according to any of SEQ ID Nos: 227, 257, and 287; a LCDR2 according to any of SEQ ID Nos: 237, 267, and GTN; a LCDR3 according to any one of SEQ ID Nos: 247, 277, and 297;
(iv) a LCDR1 according to any of SEQ ID Nos: 226, 256, and 286; a LCDR2 according to any of SEQ ID Nos: 236, 266 and DTS; a LCDR3 according to any one of SEQ ID Nos: 246, 276, and 296;
(v) a LCDR1 according to any of SEQ ID Nos: 225, 255, and 285; a LCDR2 according to any of SEQ ID Nos: 235, 265, and RAS; a LCDR3 according to any one of SEQ ID Nos: 245, 275, and 295;
(vi) a LCDR1 according to any of SEQ ID Nos: 224, 254, and 284; a LCDR2 according to any of SEQ ID Nos: 234, 264, and GTN; a LCDR3 according to any one of SEQ ID Nos: 244, 274, and 294;
(vii) a LCDR1 according to any of SEQ ID Nos: 223, 253, and 283; a LCDR2 according to any of SEQ ID Nos: 233, 263, and SGS; a LCDR3 according to any one of SEQ ID Nos: 243, 273, and 293;
(viii) a LCDR1 according to any of SEQ ID Nos: 229, 259, and 289; a LCDR2 according to any of SEQ ID Nos: 239, 269, and GTN; a LCDR3 according to any one of SEQ ID Nos: 249, 279, and 299;
(ix) a LCDR1 according to any of SEQ ID Nos: 231, 261 and 291; a LCDR2 according to any of SEQ ID Nos: 241, 271, and GTN; a LCDR3 according to any one of SEQ ID Nos: 251, 281, and 301; or
(x) a LCDR1 according to any of SEQ ID Nos: 230, 260, 290; a LCDR2 according to any of SEQ ID Nos: 240, 270, and GTN; a LCDR3 according to any one of SEQ ID Nos: 250, 280, and 300.

In certain aspects, the GALV gp70 antigen binding system, antibody, or antigen binding fragment comprises a first domain comprising three heavy chain complementarity determining regions (HCDRs) and a second domain comprising three light chain complementarity determining regions (LCDRs), wherein:

the HCDRs and LCDRs comprise:
(i) a HCDR1 according to any of SEQ ID Nos: 127, 157, and 187; a HCDR2 according to any of SEQ ID Nos: 139, 169, and 199; a HCDR3 according to SEQ ID NO: 211 or DYY; a LCDR1 according to any of SEQ ID Nos: 232, 262, and 292; a LCDR2 according to any of SEQ ID Nos: 242, 272 and GTN; a LCDR3 according to any one of SEQ ID Nos: 252, 282, and 302;
(ii) a HCDR1 according to any of SEQ ID Nos: 128, 158 and 188; a HCDR2 according to any of SEQ ID Nos: 140, 170, and 200; a HCDR3 according to any one of SEQ ID Nos: 151, 181, and 212; a LCDR1 according to any of SEQ ID Nos: 228, 258, and 288; a LCDR2 according to any of SEQ ID Nos: 238, 268, and KVS; a LCDR3 according to any one of SEQ ID Nos: 248, 278, and 298;
(iii) a HCDR1 according to any of SEQ ID Nos: 129, 159, and 189; a HCDR2 according to any of SEQ ID Nos: 141, 171, and 201; a HCDR3 according to any one of SEQ ID Nos: 152, 182, and 213; a LCDR1 according to any of SEQ ID Nos: 226, 256, and 286; a LCDR2 according to any of SEQ ID Nos: 236, 266 and DTS; a LCDR3 according to any one of SEQ ID Nos: 246, 276, and 296
(iv) a HCDR1 according to any of SEQ ID Nos: 130, 160, and 190; a HCDR2 according to any of SEQ ID Nos: 142, 172, and 202; a HCDR3 according to SEQ ID NO: 214 or DYY; a LCDR1 according to any of SEQ ID Nos: 227, 257, and 287; a LCDR2 according to any of SEQ ID Nos: 237, 267, and GTN; a LCDR3 according to any one of SEQ ID Nos: 247, 277, and 297;
(v) a HCDR1 according to any of SEQ ID Nos: 131, 161, and 191; a HCDR2 according to any of SEQ ID Nos: 143, 173, and 203; a HCDR3 according to any one of SEQ ID Nos: 153, 183, and 215; a LCDR1 according to any of SEQ ID Nos: 227, 257, and 287; a LCDR2 according to any of SEQ ID Nos: 237, 267, and GTN; a LCDR3 according to any one of SEQ ID Nos: 247, 277, and 297;
(vi) a HCDR1 according to any of SEQ ID Nos: 132, 162 and 192; a HCDR2 according to any of SEQ ID Nos: 144, 174 and 204; a HCDR3 according to any one of SEQ ID Nos: 154, 184, and 216; a LCDR1 according to any of SEQ ID Nos: 226, 256, and 286; a LCDR2 according to any of SEQ ID Nos: 236, 266 and DTS; a LCDR3 according to any one of SEQ ID Nos: 246, 276, and 296;
(vii) a HCDR1 according to any of SEQ ID Nos: 133, 163, and 193; a HCDR2 according to any of SEQ ID Nos: 145, 175, and 205; a HCDR3 according to any one of SEQ ID Nos: 155, 185, and 217; a LCDR1 according to any of SEQ ID Nos: 225, 255, and 285; a LCDR2 according to any of SEQ ID Nos: 235, 265, and RAS; a LCDR3 according to any one of SEQ ID Nos: 245, 275, and 295;
(viii) a HCDR1 according to any of SEQ ID Nos: 134, 164, and 194; a HCDR2 according to any of SEQ ID Nos: 146, 176, and 206; a HCDR3 according to SEQ ID NO: 218 or DYY; a LCDR1 according to any of SEQ ID Nos: 224, 254, and 284; a LCDR2 according to any of SEQ ID Nos: 234, 264, and GTN; a LCDR3 according to any one of SEQ ID Nos: 244, 274, and 294
(ix) a HCDR1 according to any of SEQ ID Nos: 135, 165, and 195; a HCDR2 according to any of SEQ ID Nos: 147, 177, and 207; a HCDR3 according to any one of SEQ ID Nos: 156, 186 and 219; a LCDR1 according to any of SEQ ID Nos: 223, 253, and 283; a LCDR2 according to any of SEQ ID Nos: 233, 263, and SGS; a LCDR3 according to any one of SEQ ID Nos: 243, 273, and 293;
(x) a HCDR1 according to any one of SEQ ID Nos: 136, 166, and 196; a HCDR2 according to any of SEQ ID Nos: 148, 178, and 208; a HCDR3 according to SEQ ID NO: 220 or DYY; a LCDR1 according to any of SEQ ID Nos: 229, 259, and 289; a LCDR2 according to any of SEQ ID Nos: 239, 269, and GTN; a LCDR3 according to any one of SEQ ID Nos: 249, 279, and 299;
(xi) a HCDR1 according to any one of SEQ ID Nos: 137, 167, and 197; a HCDR2 according to any of SEQ ID Nos: 149, 179, and 209; a HCDR3 according to SEQ ID NO: 221 or DYY; a LCDR1 according to any of SEQ ID Nos: 231, 261 and 291; a LCDR2 according to any of SEQ ID Nos: 241, 271, and GTN; a LCDR3 according to any one of SEQ ID Nos: 251, 281, and 301; or
(xii) a HCDR1 according to any one of SEQ ID Nos: 138, 168, and 198; a HCDR2 according to any of SEQ ID Nos: 150, 180, and 210; a HCDR3 according to SEQ ID NO: 222 or DYY; a LCDR1 according to any of SEQ ID Nos: 230, 260, 290; a LCDR2 according to any of SEQ ID Nos: 240, 270, and GTN; a LCDR3 according to any one of SEQ ID Nos: 250, 280, and 300.

In certain aspects, the GALV gp70 antigen binding system, antibody, or antigen binding fragment thereof comprises a first heavy chain variable domain comprising three HCDRs and a light chain variable domain comprising three LCDRs, wherein:
(i) the heavy chain variable domain is at least 80% identical to any one of SEQ ID Nos: 303-314; and
(ii) the light chain variable domain is at least 80% identical to any one of SEQ ID Nos: 315-324.

In certain aspects, the GALV gp70 antigen binding system, antibody, or antigen binding fragment thereof comprises a first heavy chain variable domain comprising the three HCDRs and a light chain variable domain comprising the three LCDRs, wherein:
(i) the heavy chain variable domain is at least 80% identical to SEQ ID NO: 303 and the light chain variable domain is at least 80% identical to SEQ ID NO: 324;
(ii) the heavy chain variable domain is at least 80% identical to SEQ ID NO: 304 and the light chain variable domain is at least 80% identical to SEQ ID NO: 320;
(iii) the heavy chain variable domain is at least 80% identical to SEQ ID NO: 305 and the light chain variable domain is at least 80% identical to SEQ ID NO: 318;
(iv) the heavy chain variable domain is at least 80% identical to SEQ ID NO: 306 and the light chain variable domain is at least 80% identical to SEQ ID NO: 319;
(v) the heavy chain variable domain is at least 80% identical to SEQ ID NO: 307 and the light chain variable domain is at least 80% identical to SEQ ID NO: 319;
(vi) the heavy chain variable domain is at least 80% identical to SEQ ID NO: 308 and the light chain variable domain is at least 80% identical to SEQ ID NO: 318;
(vii) the heavy chain variable domain is at least 80% identical to SEQ ID NO: 309 and the light chain variable domain is at least 80% identical to SEQ ID NO: 317;
(viii) the heavy chain variable domain is at least 80% identical to SEQ ID NO: 310 and the light chain variable domain is at least 80% identical to SEQ ID NO: 316;
(ix) the heavy chain variable domain is at least 80% identical to SEQ ID NO: 311 and the light chain variable domain is at least 80% identical to SEQ ID NO: 315;
(x) the heavy chain variable domain is at least 80% identical to SEQ ID NO: 312 and the light chain variable domain is at least 80% identical to SEQ ID NO: 321;
(xi) the heavy chain variable domain is at least 80% identical to SEQ ID NO: 313 and the light chain variable domain is at least 80% identical to SEQ ID NO: 323; or
(xii) the heavy chain variable domain is at least 80% identical to SEQ ID NO: 314 and the light chain variable domain is at least 80% identical to SEQ ID NO: 322.

In certain aspects, the GALV gp70 antigen binding system, antibody, or antigen binding fragment thereof is characterized wherein the three HCDRs and the three LCDRs are comprised by a single polypeptide.

In certain aspects, the GALV gp70 antigen binding system, antibody, or antigen binding fragment thereof is characterized wherein the three HCDRs are comprised by a first polypeptide and the three LCDRs are comprised by a second polypeptide.

In certain aspects, the GALV gp70 antigen binding system, antibody, or antigen binding fragment thereof is characterized, wherein the first polypeptide is an antibody heavy chain and the second polypeptide is an antibody light chain.

In certain aspects, a nucleic acid is disclosed encoding at least one GALV gp70 binding polypeptide as described above.

In certain aspects, a vector is disclosed comprising such a nucleic acid.

In certain aspects, a method of generating an engineered cell is disclosed, wherein the method comprises transfecting or transducing a cell with a nucleic acid or a vector as described immediately above.

In certain aspects, a cell encoding or expressing a GALV gp70 antigen binding system, antibody, or antigen binding fragment thereof is disclosed, optionally wherein the cell is an immune cell.

In certain aspects, the GALV gp70 antigen binding system, antibody, or antigen binding fragment thereof, further comprises a detectable label.

In certain aspects, the GALV gp70 antigen binding system, antibody, or antigen binding fragment thereof comprises a detectable label selected from the group consisting of a fluorescent label, a photochromic compound, a proteinaceous fluorescent label, a magnetic label, a radiolabel, and a hapten.

In certain aspects, the GALV gp70 antigen binding system, antibody, or antigen binding fragment thereof comprises a detectable label which is a fluorescent label selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreen1, TagYFP, EYFP, Topaz, Venus, mCitrine, Ypet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoerythrin (BPE), mCherry, HcRed1, Katusha, P3, Peridinin Chlorophyll (PerCP), mKate (TagFP635), TurboFP635, mPlum, and mRaspberry.

In certain aspects, a method for determining the number of viral particles expressing a Gibbon ape leukemia virus (GALV) gp70 protein having the amino acid sequence of SEQ ID NO: 325 is disclosed, the method comprising:
(a) providing a sample known or suspected to comprise viral particles expressing the GALV gp70 protein;
(b) contacting the sample with an antigen binding system, antibody, or antigen binding fragment thereof of any one of claims 21 to 29 wherein the antigen binding system, antibody, or antigen binding fragment thereof further comprises a detectable label, under conditions that permit the formation of one or more binding complex comprising a viral particle and the antigen binding system, antibody, or antigen binding fragment thereof;
(c) detecting the one or more binding complexes by detection of the detectable label and
(d) determining the number of viral particles present in the sample based on the detection of step (c).

In certain aspects, a method of determining the presence or absence of viral particles expressing a Gibbon ape leukemia virus (GALV) gp70 protein having the amino acid sequence of SEQ ID NO: 325 is disclosed, the method comprising:
(a) providing a sample known or suspected to comprise viral particles expressing the GALV gp70 protein;
(b) providing an antigen binding molecule that specifically binds the GALV gp70 protein, wherein the antigen binding molecule further comprises a detectable label;
(c) contacting the sample with the antigen binding molecule under conditions that permit the formation of a binding complex between GALV gp70 and the antigen binding protein;
(d) separating any molecules not part of a binding complex from the binding complexes; and
(e) detecting the presence or absence of a binding complex.

In certain aspects, the method for determining the number of viral particles or the method of determining the presence or absence of viral particles is characterized, wherein the antigen binding molecule is disposed on a surface selected from the group consisting of an agarose bead, a magnetic bead, a plastic welled plate, a glass welled plate, a ceramic welled plate and a cell culture bag.

In certain aspects, the method for determining the number of viral particles or the method of determining the presence or absence of viral particles is characterized, wherein the detectable label is selected from the group consisting of a fluorescent label, a photochromic compound, a proteinaceous fluorescent label, a magnetic label, a radiolabel, and a hapten.

In certain aspects, the method for determining the number of viral particles or the method of determining the presence or absence of viral particles is characterized, wherein the fluorescent label is selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreen1, TagYFP, EYFP, Topaz, Venus, mCitrine, Ypet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoerythrin (BPE), mCherry, HcRed1, Katusha, P3, Peridinin Chlorophyll (PerCP), mKate (TagFP635), TurboFP635, mPlum, and mRaspberry.

In certain aspects, the method for determining the number of viral particles or the method of determining the presence or absence of viral particles is characterized, wherein the detecting is performed with a flow based detection method.

In certain aspects, the method for determining the number of viral particles or the method of determining the presence or absence of viral particles is characterized, wherein the flow based detection method is flow virometry.

In certain aspects, the method for determining the number of viral particles or the method of determining the presence or absence of viral particles is characterized, wherein the detecting is performed by ELISA, bio-layer interferometry (BLI), Western blot or any combination thereof.

### BRIEF DESCRIPTION OF THE FIGURES

FIG. 1, panel A-E is experimental data for controls for expression data by transduced T cells.

FIG. 2 shows experimental data showing scFv14-bearing CAR binding as compared to controls.

FIG. 3, panel A shows experimental data for binding of supernatants to scFv14 for likely hybridoma candidates.

FIG. 3, panel B shows experimental data for binding of supernatants to scFv14 for likely hybridoma candidates.

FIG. 4 shows experimental data that evaluated the specificity to scFv14. Supernatants were screened for binding to T cells expressing the scFv14-bearing CAR, the irrelevant anti-CD20 scFv2-bearing or Leu16-bearing CARs or NTD T cell controls.

FIG. 5 shows experimental data that evaluated scFv14 specificity.

FIG. 6, panel A shows experimental data for clone 24C12 conjugated to PE screened against healthy donor T cells that were left NTD or transduced to express CARs bearing either scFv2, scFv14, or FMC63.

FIG. 6, panel B shows experimental data for clone 24C12 conjugated to FITC screened against healthy donor T cells that were left NTD or transduced to express CARs bearing either scFv2, scFv14, or FMC63.

FIG. 7, panel A shows experimental data for clone 24C12 conjugated to PE screened against healthy donor T cells that were left NTD or transduced to express CARs bearing either scFv2, scFv14, or FMC63.

FIG. 7, panel B shows experimental data for clone 24C12 conjugated to FITC screened against healthy donor T cells that were left NTD or transduced to express CARs bearing either scFv2, scFv14, or FMC63.

FIG. 8 shows a schematic of recombinant GALV gp70 viral envelope protein designs. A secretion signal (SS) was included on all constructs. Various affinity tags (monoFc huIgG1, muIgG2a Fc or 6×His (SEQ ID NO: 348)) were included for purification. The entire predicted viral surface exposed portion of GALV gp70 was made including residues 42-616 (numbering according to Uniprot P21415), with an N-terminal affinity tag. Truncations including the coiled-coil region (residues 505-616) or the receptor binding domain only (residues 42-474) were made with C-terminal affinity tags.

FIG. 9A shows K562 or CHO cells stained with anti-SLC20A1 antibody (Proteintech cat #12423-1-AP) and a PE-conjugated secondary antibody. Cells were stained with the secondary antibody alone as a control. The percent of cells positive for SCL20A1 surface expression are shown (94.7% for K562 and 0% for CHO). FIG. 9B shows K562 or CHO cells stained with a dilution series of recombinant GALV gp70 proteins followed by a PE-conjugated secondary antibody. PE mean fluorescence intensity (MFI) increased in a dose-dependent manner when K562 cells were stained with gp70 (42-474) or (42-616), but not (505-616). No staining was observed for the CHO cells.

FIG. 10A shows indirect ELISA titers using immobilized GALV gp70 (42-616) protein measured for immunized mouse sera or normal mouse sera as a negative control. The two mice (Ms6609 and Ms6423) with the highest OD450 reads were chosen for hybridoma fusions. FIG. 10B shows PG13 cells stably producing viral particles containing the GALV gp70 envelope protein were stained with a dilution series of serum from immunized mice chosen for hybridoma fusion and a PE conjugated secondary antibody (left and middle). Negative controls including no staining (NS), normal mouse serum (non-immunized mice: NMS1 and NMS2), or an isotype control antibody staining of PG13 cells, are shown (right)

FIG. 11 shows the relative comparison of indirect ELISA titers using immobilized GALV gp70 proteins (42-616), (505-616), (42-616) or an affinity tagged, negative control protein, measured after incubation with hybridoma supernatants. The 17 positive hybridoma clones are shown.

FIG. 12 shows PG13 cells stably producing viral particles containing the GALV gp70 envelope protein incubated with a dilution series of each hybridoma supernatant and stained with a PE-conjugated secondary antibody. The relative PE signal for each hybridoma is shown.

FIG. 13 shows PG13 cells (top) stably producing viral particles containing the GALV gp70 envelope protein were incubated with a single dilution ( 1/10) of each antibody clone successfully purified at small scale. NIH-3T3 cells (bottom) were stained with the same antibody clone as a negative control.

FIGS. 14A-14I show PG13 cells (left) stably producing viral particles containing the GALV gp70 envelope protein were incubated with a dilution series of each antibody clone purified at large scale (top concentration of 10 ug/mL down to 0.005 ug/mL). NIH-3T3 cells (right) were stained with the same antibody clone dilution series as a negative control. Isotype controls were also included for both cell line stains at the top concentration of 10 ug/mL.

FIGS. 15A-15H show purified antibody clones loaded onto AMC biosensors (2 ug/mL). Octet sensorgrams (association and disassociation) are shown for all clones demonstrating binding to 100 nM monoFc tagged GALV gp70 protein residues 42-474).

FIG. 16 shows hybridoma supernatants which do not bind to FMC63-28z at the highest concentration tested. The sample identity is indicated in the left side of each panel. Each plot shows a histogram of fluorescence intensity values (x-axis) plotted against their frequencies normalized to their respective modes (y-axis).

### DETAILED DESCRIPTION OF THE DISCLOSURE

In certain aspects, present embodiments relate to anti-idiotypic antigen binding molecules, including antibodies, which specifically bind to antigen binding molecules that specifically bind to anti-CD20 scFv14 (see, Kanyarat Thueng-in, Jeeraphong Thanongsaksrikul, Surasak Jittavisutthikul, Watee Seesuay, Monrat Chulanetra, Yuwaporn Sakolvaree, Potjanee Srimanote & Wanpen Chaicumpa (2014) Interference of HCV replication by cell penetrable human monoclonal scFv specific to NS5B polymerase, mAbs, 6:5, 1327-1339, DOI: 10.4161/mabs.29978).

The anti-CD20 scFv-14 has the amino acid sequence:

VH DNA -
(SEQ ID NO: 122)
CAGGTTCAGCTGGTGCAGTCTGGAGCTGAGGTGAAGAAGCCTGGGGCCTC
AGTGAAGGTCTCCTGCAAGGCTTCTGGTTACACCTTTAAAGAATATGGTA
TCAGCTGGGTGCGACAGGCCCCTGGACAAGGGCTTGAGTGGATGGGATGG
ATCAGCGCTTACAGTGGTCACACATACTATGCACAGAAGCTCCAGGGCAG
AGTCACCATGACCACAGACACATCCACGAGCACAGCCTACATGGAGCTGA
GGAGCCTGAGATCTGACGACACGGCGGTGTACTACTGCGCCAGAGGGCCT
CACTACGACGACTGGAGCGGATTTATCATATGGTTCGACCCATGGGGACA
GGGTACATTGGTCACCGTCTCCTCA
VL DNA -
(SEQ ID NO: 123)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCT
GCATCCAGTTTGCAAAGTGGGGTCCCTTCAAGGTTCAGTGGCAGTGGATC
TGGGACAGATTTCACTCTCACCATCAGCAGTCTGCAACCTGAAGATTTTG
CAACTTACTACTGTCAACAGAGTTACAGGTTTCCTCCTACCTTTGGCCAA
GGGACCAAGGTTGAGATCAAA
VH Protein -
(SEQ ID NO: 124)
QVQLVQSGAEVKKPGASVKVSCKASGYTFKEYGISWVRQAPGQGLEWMGW
ISAYSGHTYYAQKLQGRVTMTTDTSTSTAYMELRSLRSDDTAVYYCARGP
HYDDWSGFIIWFDPWGQGTLVTVSS
VL Protein -
(SEQ ID NO: 125)
DIQMTQSPSSLSASVGDRVTITCRASQSISSYLNWYQQKPGKAPKLLIYA
ASSLQSGVPSRFSGSGSGTDFTLTISSLQPEDFATYYCQQSYRFPPTFGQ
GTKVEIK

Humanized forms of the antigen binding molecules, molecules comprising the anti-CD20 scFv14 and cells presenting a molecule comprising the anti-CD20 scFv14 are also provided. Additionally, polynucleotides encoding the antigen binding molecules, as well as vectors comprising the polynucleotides, and in vitro cells comprising the polynucleotides and vectors, are also disclosed.

Methods of using the disclosed antigen binding molecules are provided. The antigen binding molecules, polynucleotides, vectors, in vitro cells and methods described herein can be used in a range of applications, e.g., as reagents to detect the presence of moieties comprising the anti-CD20 scFv14, as well as molecules comprising this sequence and cells presenting such molecules, quantifying the amount of a moiety comprising anti-CD20 scFv14, as well as molecules comprising this sequence and cells presenting such molecules, screening for moieties comprising the anti-CD20 scFv14, as well as molecules comprising this sequence and cells presenting such molecules, purifying moieties comprising the anti-CD20 scFv14, as well as molecules comprising this sequence and cells presenting such molecules, and biomarker studies focused on moieties comprising the anti-CD20 scFv, as well as molecules comprising this sequence and cells presenting such molecules. Therapeutic uses are also provided, for example applications in which the biological activity of a moiety comprising the anti-CD20 scFv14, as well as cells presenting such molecules, is modulated (enhanced or repressed), as well as dose ranging studies related to therapeutics comprising the anti-CD20 scFv14, as well as molecules comprising this sequence and cells presenting such molecules, and cells presenting such molecules.

The antigen binding molecules (e.g., scFvs, antibodies, etc.) disclosed herein were generated from hybridomas generated using B-cells of mouse origin, but can be readily humanized using standard methods known to those of skill in the art, as well as those described herein. Representative humanized forms of the disclosed antigen binding molecules can be generated as described herein.

In certain further aspects, the present embodiments relate to antigen binding molecules, including antibodies, which specifically bind to a viral coat protein. In certain aspects, the antigen binding molecules bind to Gibbon ape leukemia virus gp70 envelop protein (GALV gp70). GALV gp70 is a surface protein which attaches the virus to a host cell by binding to its receptor.

GALV gp70 has the amino acid sequence:

(SEQ ID NO: 325)
MVLLPGSMLLTSNLHHLRHQMSPGSWKRLIILLSCVFGGGGTSLQNKNPH
QPMTLTWQVLSQTGDVVWDTKAVQPPWTWWPTLKPDVCALAASLESWDIP
GTDVSSSKRVRPPDSDYTAAYKQITWGAIGCSYPRARTRMASSTFYVCPR
DGRTLSEARRCGGLESLYCKEWDCETTGTGYWLSKSSKDLITVKWDQNSE
WTQKFQQCHQTGWCNPLKIDFTDKGKLSKDWITGKTWGLRFYVSGHPGVQ
FTIRLKITNMPAVAVGPDLVLVEQGPPRTSLALPPPLPPREAPPPSLPDS
NSTALATSAQTPTVRKTIVTLNTPPPTTGDRLFDLVQGAFLTLNATNPGA
TESCWLCLAMGPPYYEAIASSGEVAYSTDLDRCRWGTQGKLTLTEVSGHG
LCIGKVPFTHQHLCNQTLSINSSGDHQYLLPSNHSWWACSTGLTPCLSTS
VFNQTRDFCIQVQLIPRIYYYPEEVLLQAYDNSHPRTKREAVSLTLAVLL
GLGITAGIGTGSTALIKGPIDLQQGLTSLQIAIDADLRALQDSVSKLEDS
LTSLSEVVLQNRRGLDLLFLKEGGLCAALKEECCFYIDHSGAVRDSMKKL
KEKLDKRQLERQKSQNWYEGWFNNSPWFTTLLSTIAGPLLLLLLLLILGP
CIINKLVQFINDRISAVKILVLRQKYQALENEGNL.

Additionally, polynucleotides encoding the GALV gp70 binding molecules, as well as vectors comprising the polynucleotides, and in vitro cells comprising the polynucleotides and vectors, are also disclosed.

Methods of using the disclosed antigen binding molecules are provided. The antigen binding molecules, polynucleotides, vectors, in vitro cells and methods described herein can be used in a range of applications, e.g., as reagents to detect the presence of moieties comprising the GALV gp70, as well as molecules comprising this sequence and cells, as well as viral particles, presenting such molecules, quantifying the amount of a moiety comprising GALV gp70, as well as molecules comprising this sequence and cells presenting such molecules, screening for moieties comprising GALV gp70, as well as molecules comprising this sequence and cells presenting such molecules, purifying moieties comprising the GALV gp70, as well as molecules comprising this sequence and cells presenting such molecules, and biomarker studies focused on moieties comprising GALV gp70, as well as molecules comprising this sequence and cells presenting such molecules. Therapeutic uses are also provided, for example applications in which the biological activity of a moiety comprising the GALV gp70, as well as cells presenting such molecules, is modulated (enhanced or repressed), as well as dose ranging studies related to therapeutics comprising GALV gp70, as well as molecules comprising this sequence and cells presenting such molecules, and cells presenting such molecules.

### I. Definitions

In order that the present disclosure may be more readily understood, certain terms are first defined. As used in this application, except as otherwise expressly provided herein, each of the following terms shall have the meaning set forth below. Additional definitions are set forth throughout the application. The headings provided herein are not limitations of the various aspects of the disclosure, which aspects should be understood by reference to the specification as a whole.

It is understood that, wherever aspects are described herein with the language “comprising,” otherwise analogous aspects described in terms of “consisting of” and/or “consisting essentially of” are also provided.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, The Concise Dictionary of Biomedicine and Molecular Biology, 2^{nd}ed., (2001), CRC Press; The Dictionary of Cell & Molecular Biology, 5^{th} ed., (2013), Academic Press; and The Oxford Dictionary Of Biochemistry And Molecular Biology, Cammack et al. eds., 2^{nd} ed, (2006), Oxford University Press, provide those of skill in the art with a general dictionary for many of the terms used in this disclosure.

As used herein, the twenty conventional (e.g., naturally occurring) amino acids and their abbreviations follow conventional usage. See, e.g., Immunolo-y—A Synthesis (2nd Edition), Golub and Green, eds., Sinauer Assoc., Sunderland, Mass. (1991), which is incorporated herein by reference for any purpose. Stereoisomers (e.g., D-amino acids) of the twenty conventional amino acids, unnatural amino acids such as alpha-, alpha-disubstituted amino acids, N-alkyl amino acids, lactic acid, and other unconventional amino acids can also be suitable components for polypeptides of the present invention. Examples of unconventional amino acids include: 4-hydroxyproline, gamma-carboxyglutamate, epsilon-N,N,N-trimethyllysine, e-N-acetyllysine, O-phosphoserine, N-acetylserine, N-formylmethionine, 3-methylhistidine, 5-hydroxylysine, sigma-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As used herein, the term the terms “a” and “an” are used per standard convention and mean one or more, unless context dictates otherwise.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. Alternatively, “about” or “comprising essentially of” can mean a range of up to 10% (i.e., ±10%). For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

As used herein, the term “and/or” is to be understood as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or,” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term the use of the alternative (e.g., “or”) should be understood to mean either one, both, or any combination thereof of the alternatives.

As used herein, the term “allogeneic” refers to any material derived from one individual which is then introduced to another individual of the same species, e.g., allogeneic T cell transplantation.

The term “antibody” includes, both naturally occurring and non-naturally occurring (recombinantly-produced) antibodies, human and non-human antibodies, monospecific antibodies, multispecific antibodies (including bispecific antibodies), immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies (see, e.g., Stocks, (2004) Drug Discovery Today 9(22):960-66), antibody fusions (which term encompasses antibody-drug conjugates) and which are sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments thereof. In certain embodiments, antibodies described herein refer to polyclonal antibody populations.

The term “antibody” also encompasses an intact immunoglobulin or an antigen binding portion thereof that competes with the intact antibody for specific binding, unless otherwise specified. Antigen binding portions can be produced by recombinant DNA techniques or by enzymatic or chemical cleavage of intact antibodies. Antigen binding portions include, inter alia, Fab, Fab′, F(ab′)2, Fv, domain antibodies (dAbs), fragments including complementarity determining regions (CDRs), single-chain antibodies (scFv), chimeric antibodies, diabodies, triabodies, tetrabodies, and polypeptides that contain at least a portion of an immunoglobulin that is sufficient to confer specific antigen binding to the polypeptide.

A non-human antibody may be humanized using recombinant methods to reduce its immunogenicity in humans, as disclosed herein, with respect to antibodies that specifically bind the anti-CD20 scFv14, as well as molecules comprising this sequence and cells presenting such molecules. Where not expressly stated, and unless the context indicates otherwise, the term “antibody” also includes an antigen-binding fragment of an antigen binding molecule of any of the aforementioned immunoglobulins, and includes a monovalent and a divalent fragment or portion, and a single chain antibody (i.e., a scFv).

As used herein, the term “antigen” means any molecule that provokes an immune response or is capable of being bound by an antibody or other antigen binding molecule. The immune response can involve either antibody production, or the activation of specific immunologically-competent cells, or both. Those of skill in the art will readily understand that any macromolecule, including virtually all proteins or peptides (including the anti-CD20 scFv14), as well as molecules comprising the same sequence and cells presenting such molecules), can serve as an antigen. Generally, an antigen can be endogenously expressed, i.e. expressed by genomic DNA, or it can be recombinantly expressed, or it can be chemically synthesized. In one particular embodiment, an antigen comprises all or a portion of the anti-CD20 scFv14, as well as molecules comprising the same sequence, which is optionally conjugated to an adjuvant such as keyhole limpet hemocyanin (KLH), or to an Fc to facilitate screening.

As used herein, the term “antigen binding molecule” means a protein comprising a portion that binds to an antigen or target protein and, optionally, a scaffold or framework portion that allows the antigen binding portion to adopt a conformation that promotes binding of the antigen binding molecule to the antigen. Examples of the representative types of antigen binding molecules include a scFv, a human, mouse or rabbit antibody; a humanized antibody; a chimeric antibody; a recombinant antibody; a single chain antibody; a diabody; a triabody; a tetrabody; a Fab fragment; a F(ab′)2 fragment; an IgD antibody; an IgE antibody; an IgM antibody; an IgG1 antibody; an IgG2 anti-body; an IgG3 antibody; or an IgG4 antibody, and fragments thereof.

An antigen binding molecule can comprise, for example, an alternative protein scaffold or artificial scaffold with grafted complementarity determining regions (CDRs) or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, e.g., Korndorfer et al., 2003, Proteins: Structure, Function, and Bioinformatics, 53(1):121-129 (2003); Roque et al., Biotechnol. Prog. 20:639-654 (2004). In addition, peptide antibody mimetics (“PAMs”) can be used, as well as scaffolds based on antibody mimetics utilizing various components (e.g., fibronectin) as a scaffold. An antigen binding molecule can have, for example, the structure of a naturally occurring immunoglobulin.

An antigen binding molecule can have one or more binding sites. If there is more than one binding site, the binding sites can be identical to one another or they can be different. For example, a naturally occurring human immunoglobulin typically has two identical binding sites, while a “bispecific” or “bifunctional” antibody has two different binding sites, and is capable of specifically binding two different antigens (e.g., the anti-CD20 scFv14 and a cell surface activator molecule).

In various embodiments, an antigen binding molecule is an antibody or fragment thereof, including one or more of the complementarity determining regions (CDRs) disclosed herein, which specifically bind the anti-CD20 scFv14, as well as molecules comprising the anti-CD20 scFv14, and cells presenting such molecules. In further embodiments, the antigen binding molecule binds to a CAR comprising the anti-CD20 scFv14, as well as molecules comprising the anti-CD20 scFv14, and can be expressed on an immune cell, such as a T cell.

The term “autologous” refers to any material derived from the same individual to which it is later to be re-introduced. For example, the engineered autologous cell therapy (eACT™) methods described herein involve collection of lymphocytes from a patient, which are then engineered to express a construct, e.g., a CAR construct, and then administered back to the same patient.

As used herein, the term “binding affinity” means the strength of the sum total of non-covalent interactions between a single binding site of a molecule (e.g., an antigen binding molecule such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by standard techniques known to one of ordinary skill in the art, such as BIAcore® or KinExA or surface plasmon resonance.

As used herein, the term “complementarity determining region” or “CDR” means an amino acid sequence that contributes to antigen binding specificity and affinity. Framework regions can aid in maintaining the proper confirmation of the CDRs to promote binding between the antigen binding molecule and an antigen. A number of definitions of the CDRs are commonly in use: Kabat numbering, Chothia numbering, IMGT numbering, AbM numbering, or contact numbering. The AbM definition is a compromise between the Kabat and Chothia systems, and is used by Oxford Molec'lar's AbM antibody modelling software. Table 1 defines CDRs using each numbering system. The contact definition is based on an analysis of the available complex crystal structures.

TABLE 1

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26--H32 . . . 34 | H30--H35B |
| H1 | H31--H35 | H26--H35 | H26--H32 | H30--H35 |
| H2 | H50--H65 | H50--H58 | H52--H56 | H47--H58 |
| H3 | H95--H102 | H95--H102 | H95--H102 | H93--H101 |

The term “Kabat numbering” and like terms are recognized in the art and refer to a system of numbering amino acid residues in the heavy and light chain variable regions of an antibody, or an antigen binding molecule thereof. In certain aspects, the CDRs of an antibody can be determined according to the Kabat numbering system (see, e.g., Kabat et al. in Sequences of Proteins of Immunological Interes^{t,} 5th Ed., NIH Publication 91-3242, Bethesda MD 1991).

Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some embodiments, the CDRs of the antibodies described herein can be described according to the Kabat numbering scheme, (although they can readily be construed in other numbering systems using Table 1 above). In some embodiments, the CDRs of the antibodies described herein can be described according to the Clothia numbering scheme. In some embodiments, the CDRs of the antibodies described herein can be described according to the IGMT numbering scheme.

In certain aspects, the CDRs of an antibody can be determined according to the Chothia numbering scheme, which refers to the location of immunoglobulin structural loops (see, e.g., Chothia C & Lesk AM, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). See Table 1. In some embodiments, the CDRs of the antibodies described herein have been determined according to the Chothia numbering scheme.

As used herein, a “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen binding molecule provided herein (or fragment thereof) can be replaced with an amino acid residue with a similar side chain.

Conservative amino acid substitutions, which are encompassed by the present disclosure, can encompass non-naturally occurring amino acid residues, which are typically incorporated by chemical peptide synthesis rather than by synthesis in biological systems. These include peptidomimetics and other reversed or inverted forms of amino acid moieties. Naturally occurring residues can be divided into classes based on common side chain properties:
hydrophobic: norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro; and
aromatic: Trp, Tyr, Phe.

Non-conservative substitutions can involve the exchange of a member of one of these classes for a member from another class. Such substituted residues can be introduced, for example, into regions of a human antibody that are homologous with non-human antibodies, or into the non-homologous regions of the molecule. Exemplary conservative amino acid substitutions are set forth in Table 2 below.

TABLE 2

| Original | | |
|---|---|---|
| Residues | Exemplary Substitutions | Preferred Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, | Leu |
| | Norleucine | |
| Leu | Norleucine, Ile, Val, Met, Ala, | Ile |
| | Phe | |
| Lys | Arg, 1,4 Diamino-butyric acid, | Arg |
| | Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |
| | Norleucine | |

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobin molecule generally has a more conserved amino acid sequence relative to an immunoglobin variable domain.

As used herein, the term “cross competes” means the situation in which the interaction between an antigen and a first antigen binding molecule or binding fragment thereof blocks, limits, inhibits, or otherwise reduces the ability of a reference antigen binding molecule or binding fragment thereof to interact with the antigen. Cross competition can be complete, e.g., binding of the binding molecule to the antigen completely blocks the ability of the reference binding molecule to bind the antigen, or it can be partial, e.g., binding of the binding molecule to the antigen reduces the ability of the reference binding molecule to bind the antigen. In certain embodiments, an antigen binding molecule that cross competes with a reference antigen binding molecule binds the same or an overlapping epitope as the reference antigen binding molecule. In other embodiments, the antigen binding molecule that cross competes with a reference antigen binding molecule binds a different epitope than the reference antigen binding molecule.

Numerous types of competitive binding assays can be used to determine if one antigen binding molecule competes with another, for example: solid phase direct or indirect radioimmunoassay (RIA); solid phase direct or indirect enzyme immunoassay (EIA); sandwich competition assay (Stahli et al., (1983) Method Enzymol 9:242-53); solid phase direct biotin-avidin EIA (Kirkland et al., (1986) J Immunol 137:3614-19); solid phase direct labeled assay, solid phase direct labeled sandwich assay (Harlow and Lane, 1988, Antibodies, A Laboratory Manual, Cold Spring Harbor Press); solid phase direct label RIA using I¹²⁵ label (Morel et al., (1988) Molec Immunol 25:7-15); solid phase direct biotin-avidin EIA (Cheung et al., (1990) Virology 176:546-52); and direct labeled RIA (Moldenhauer et al., (1990) Scand J Immunol 32:77-82).

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding molecule (a derivative) can have a greater circulating half-life than an antigen binding molecule that is not chemically modified. In some embodiments, a derivative antigen binding molecule is covalently modified to include one or more water soluble polymer attachments, including, but not limited to, polyethylene glycol, polyoxyethylene glycol, or polypropylene glycol.

As used herein, the term “diabody” or dAB means bivalent antibodies comprising two polypeptide chains, wherein each polypeptide chain comprises VH and VL domains joined by a linker that is too short to allow for pairing between two domains on the same chain, thus allowing each domain to pair with a complementary domain on another polypeptide chain (see, e.g., Holliger et al., (1993) Proc Natl Acad Sci U.S.A. 90:6444-48, Poljak et al., (1994) Structure 2: 1121-23, and Perisic et al., (1994) Structure 2(12): 1217-26). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege et al., (1994) Acta Crystallogr D Biol Crystallogr 50(Pt 4): 339-350; McPherson, (1990) Eur J Biochem 189: 1-23; Chayen, (1997) Structure 5: 1269-1274; McPherson, (1976) J Biol Chem 251: 6300-6303). Antibody:antigen crystals can be studied using well known X-ray diffraction techniques and may be refined using computer software such as X-PLOR (Yale University, 1992, distributed by Molecular Simulations, Inc.; see, e.g., Meth Enzymol (1985) Vols 114 & 115, eds Wyckoff et al.,), and BUSTER (Bricogne, (1993) Acta Crystallogr D Biol Crystallogr 49(Pt 1): 37-60; Bricogne, (1997) Meth Enzymol 276A: 361-423, ed. Carter; Roversi et al., (2000) Acta Crystallogr D Biol Crystallogr 56(Pt 10): 1316-1323). Mutagenesis mapping studies can be accomplished using any method known to one of skill in the art. See, e.g., Champe et al., (1995) J Biol Chem 270: 1388-94 and Cunningham & Wells, (1989) Science 244: 1081-85 for a description of mutagenesis techniques, including alanine and arginine scanning mutagenesis techniques.

As used herein, the term “Fab fragment” means is a monovalent fragment having the VL, VH, CL and CH domains; a “F(ab′)2 fragment” is a bivalent fragment having two Fab fragments linked by a disulfide bridge at the hinge region; a “Fv fragment” has the VH and VL domains of a single arm of an antibody; and a “dAb fragment” has a VH domain, a VL domain, or an antigen-binding fragment of a VH or VL domain.

As used herein, the terms “immunospecifically binds,” “immunospecifically recognizes,” “specifically binds,” and “specifically recognizes” are analogous terms and are used interchangeably in the context of antigen binding molecules, and means that a given molecule preferentially binds to an antigen (e.g., epitope or immune complex) as such binding is understood by one skilled in the art. For example, an antigen binding molecule that specifically binds to an antigen may bind to other peptides or polypeptides, but with a comparatively lower affinity as determined by, e.g., immunoassays, BIAcore®, KinExA 3000 instrument (Sapidyne Instruments, Boise, ID), or other assays known in the art. In some embodiments, molecules that specifically bind to an antigen bind to the antigen with a K_{A} that is at least 2 logs, 2.5 logs, 3 logs, 4 logs or greater than the K_{A} when the molecules bind to another antigen.

In another embodiment, molecules that specifically bind to an antigen (e.g., the anti-CD20 scFv14), as well as molecules comprising the same sequence and cells presenting such molecules) bind with a dissociation constant (K_{d}) of about 1×10⁻⁷ M. In some embodiments, the antigen binding molecule specifically binds an antigen (e.g., the anti-CD20 scFv14, as well as molecules comprising the same sequence and cells presenting such molecules) with “high affinity” when the K_{d} is about 1×10⁻⁹ M to about 5×10⁻⁹ M. In some embodiments, the antigen binding molecule specifically binds an antigen (e.g., the anti-CD20 scFv14, as well as molecules comprising the same sequence and cells presenting such molecules) with “very high affinity” when the K_{d} is 1×10⁻¹⁰ M to about 5×10⁻¹⁰ M.

In still another embodiment, molecules that specifically bind to an antigen (e.g., the anti-CD20 scFv14, as well as molecules comprising this sequence and cells presenting such molecules) do not cross react with other proteins under similar binding conditions. In some embodiments, molecules that specifically bind to an antigen (e.g., the anti-CD20 scFv14, as well as molecules comprising the same sequence and cells presenting such molecules) do not cross react with other proteins that do not comprise the anti-CD20 scFv14, molecules comprising this sequence and cells presenting such molecules. In some embodiments, provided herein is an antibody or fragment thereof that binds to the anti-CD20 scFv14, as well as molecules comprising the same sequence and cells presenting such molecules, with higher affinity than to another unrelated antigen. In certain embodiments, provided herein is an antigen binding molecule (e.g., an antibody) or fragment thereof that binds to the anti-CD20 scFv14, as well as molecules comprising this sequence and cells presenting such molecules, with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In some embodiments, the extent of binding of an antigen binding molecule, antibody or antigen binding fragment thereof that specifically binds the anti-CD20 scFv14, as well as molecules comprising this sequence and cells presenting such molecules, described herein compared to an unrelated protein which does not comprise the anti-CD20 scFv14, as well as molecules comprising this sequence and cells presenting such molecules, is less than 10%, 15%, or 20% of the binding of the antibody to linker fragment protein as measured by, e.g., a radioimmunoassay.

As used herein, the term “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

As used herein, the term “immunoglobulin” means an immune molecule from any of the commonly known isotypes, including but not limited to IgA, secretory IgA, IgG and IgM. IgG subclasses are also well known to those in the art and include but are not limited to human IgG1, IgG2, IgG3 and IgG4. Many of the molecules described herein are immunoglobulins. As used herein, “isotype” means the antibody class or subclass (e.g., IgM or IgG1) that is encoded by the heavy chain constant region genes.

An immunoglobulin is a tetrameric molecule, normally composed of two identical pairs of polypeptide chains, each pair having one “light” chain (about 25 kDa) and one “heavy” chain (about 50-70 kDa). The amino-terminal portion of each chain includes a variable region of about 100 to 130 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, or IgE, respectively. Within light and heavy chains, the variable and constant regions are joined by a “J” region of about 12 or more amino acids, with the heavy chain also including a “D” region of about 10 more amino acids. See generally, Berzofsky & Berkower, Ch. 7 in Fundamental Immunology (Paul, W., ed., Lippincott Williams & Wilkins (2012); which chapter and volume is incorporated by reference in its entirety for all purposes). The variable regions of each light/heavy chain pair form the antibody binding site such that an intact immunoglobulin has two primary binding sites.

Naturally occurring immunoglobulin chains exhibit the same general structure of relatively conserved framework regions (FR) joined by three hypervariable regions, also called complementarity determining regions or “CDRs.” From N-terminus to C-terminus, both light and heavy chains comprise the domains FR1, CDR1, FR2, CDR2, FR3, CDR3 and FR4. The assignment of amino acids to each domain can be done in accordance with the definitions of Kabat (see, e.g., Kabat et al. in Sequences of Proteins of Immunological Interest, 5th Ed., NIH Publication 91-3242, Bethesda MD (1991)) or Chothia (Chothia, used herein, (see, e.g., Chothia & Lesk (1987), J. Mol. Biol. 196:901-917; Chothia et al., 1989, Nature 342:878-883 or Honegger & Pluckthun (2001), J Mol Biol 309:657-670). The Kabat, Chothia, IGMT and Abm (Oxford Molecular) numbering systems are described more fully herein.

As used herein, the term “in vitro cell” refers to any cell that is cultured ex vivo. An in vitro cell can include a human cell such as a T cell or dendritic cell, or it can include CHO, sP2/0, rabbit and other non-human cells.

As used herein, the term “light chain” when used in reference to an antibody can refer to any distinct type, e.g., kappa (κ) or lambda (λ) based on the amino acid sequence of the constant domains. Light chain amino acid sequences are known in the art. In specific embodiments, the light chain is a human light chain.

The term “neutralizing” refers to an antigen binding molecule, scFv, antibody, or a fragment thereof, that binds to a ligand (e.g., a moiety comprising the anti-CD20 scFv14, as well as molecules comprising this sequence and cells presenting such molecules) and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

As used herein, the term “patient” means any human who is being treated for an abnormal physiological condition, such as cancer or has been formally diagnosed with a disorder, those without formally recognized disorders, those receiving medical attention, those at risk of developing the disorders, etc. The terms “subject” and “patient” are used interchangeably herein and include both human and non-human animal subjects.

As used herein, the terms “peptide,” “polypeptide,” and “protein” are used interchangeably herein, and mean a compound comprising amino acid residues covalently linked by peptide bonds. A protein or peptide must contain at least two amino acids, but no limitation is placed on the maximum number of amino acids that can comprise a protein's or peptide's sequence. The term polypeptide encompasses any peptide or protein comprising two or more amino acids joined to each other by peptide bonds. As used herein, the term refers to both short chains, which also commonly are referred to as peptides, oligopeptides and oligomers, and to longer chains, which generally are referred to as proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The term “polypeptide” includes natural peptides, recombinant peptides, synthetic peptides, or a combination thereof.

In some aspects, the polypeptides and/or proteins have deletions from, additions to, and/or substitutions of one or more amino acids of antigen binding molecule. Useful polypeptide fragments may include immunologically functional fragments of antigen binding molecules, including not limited to one or more CDR regions, variable domains of a heavy and/or light chain, a portion of other portions of an antibody chain, and the like. Moieties that can be substituted for one or more amino acids of an antigen binding molecule include, e.g., D or L forms of amino acids, an amino acid different from the amino acid normally found in the same position of an antigen binding molecule, deletions, non-naturally occurring amino acids, and chemical analogs of amino acids.

Peptide analogs are commonly used in the pharmaceutical industry as non-peptide drugs with properties analogous to those of the template peptide and form an aspect of the instant disclosure. These types of non-peptide compound are termed “peptide mimetics” or “peptidomimetics.” See, e.g., Fauchere, (1986) Adv. Drug Res. (Testa, ed.) 15:29-69; Veber & Freidinger, (1985) TINS, p. 392; and Evans et al., (1987) J. Med. Chem, 30:1229-39, which are incorporated herein by reference for any purpose.

Polypeptides, peptides, proteins and analogous molecules comprising the anti-CD20 scFv14, as well as molecules comprising this sequence and cells presenting such molecules, are specifically encompassed by the terms.

As used herein, the term “percent identity” means the percent of identical residues between the amino acids or nucleotides in the compared molecules. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, ed.), (1988) New York: Oxford University Press; Biocomputing Informatics and Genome Projects, (Smith, ed.), 1993, New York: Academic Press; Computer Analysis of Sequence Data, Part I, (Griffin and Griffin, eds.), 1994, New Jersey: Humana Press; von Heinje, (1987) Sequence Analysis in Molecular Biology, New York: Academic Press; Sequence Analysis Primer, (Gribskov and Devereux, eds.), 1991, New York: M. Stockton Press; and Carillo et al., (1988) J. Applied Math. 48:1073.

In calculating percent identity, the sequences being compared are aligned in a way that gives the largest match between the sequences. The computer program used to determine percent identity can be, e.g., MOE (Chemical Computing Group) or DNASTAR (University of Wisconsin, Madison, WI). The computer algorithm GAP can be used to align the two polypeptides or polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, e.g., Dayhoff et al., (1978) Atlas of Protein Sequence and Structure 5:345-352 for the PAM 250 comparison matrix; Henikoff et al., (1992) Proc. Natl. Acad. Sci. U.S.A. 89: 10915-10919 for the BLOSUM 62 comparison matrix) is also used by the algorithm.

Certain alignment schemes for aligning two amino acid sequences can result in matching of only a short region of the two sequences, and this small aligned region can have very high sequence identity even though there is no significant relationship between the two full-length sequences. Accordingly, the selected alignment method (e.g., the GAP program) can be adjusted if desired to result in an alignment that spans at least 50 contiguous amino acids of the target polypeptide.

As used herein, the terms “single-chain antibody” and “single chain fragment variable (scFv)” are used interchangeably and mean an antigen binding molecule in which a V_{L} and a V_{H} region are joined via a linker to form a continuous protein chain wherein the linker is long enough to allow the protein chain to fold back on itself and form a monovalent antigen binding site (see, e.g., Zuhaida Asra Ahmad, Swee Keong Yeap, Abdul Manaf Ali, Wan Yong Ho, Noorjahan Banu Mohamed Alitheen, Muhajir Hamid, “scFv Antibody: Principles and Clinical Application”, Journal of Immunology Research, vol. 2012, Article ID 980250, 15 pages, 2012., which is incorporated herein by reference for any purpose in its entirety.) scFv14 is a specific example of an scFv.

A “therapeutically effective amount,” “effective dose,” “effective amount,” or “therapeutically effective dosage” of a therapeutic agent, (e.g., a moiety comprising the anti-CD20 scFv14, as well as molecules comprising this sequence and cells presenting such molecules), is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Hartl and Jones (1997) “Genetics: Principles and Analysis,” 4^{th} ed, Jones & Bartlett). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

As used herein, the terms “variable region” or “variable domain” are used interchangeably and mean a portion of an antibody, generally, a portion of a light or heavy chain, typically about the amino-terminal end of the antibody and comprising about 100-130 amino acids in the heavy chain and about 90 to 115 amino acids in the light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). The CDRs of the light and heavy chains are primarily responsible for the interaction and specificity of the antibody with antigen.

In certain embodiments, the variable region of an antigen binding molecule is a human variable region. In further embodiments, the variable region comprises rodent, human or murine CDRs and human framework regions (FRs). In further embodiments, the variable region is a primate (e.g., a non-human primate) variable region. In yet further embodiments, the variable region is a rabbit variable region. In other embodiments, the variable region comprises human CDRs and non-human (e.g., rabbit, murine, rat or non-human primate) framework regions (FRs). In other embodiments, the variable region comprises non-human (e.g., rabbit, murine, rat or non-human primate) CDRs and human framework regions (FRs).

The terms “VH,” “VH domain” and “VH chain” are used interchangeably and mean the heavy chain variable region of an antigen binding molecule, antibody or an antigen binding fragment thereof.

The terms “VL,” “VL domain” and “VL chain” are used interchangeably and mean the light chain variable region of an antigen binding molecule, antibody or an antigen binding fragment thereof.

As used herein, the term “viral particle” means one or more complete virion as well as any portion of one or more virion.

Various aspects of the invention are described in further detail in the following subsections.

### II. Overview

Immunotherapies using T cells engineered to express chimeric antigen receptors (CARs) have shown remarkable promise in the clinic with the potential to cure relapsed B-cell malignancies. However, data from multiple clinical studies have identified a critical vulnerability of treatment with anti-CD19 CAR T cells, namely the susceptibility of tumor cells to antigen escape (i.e., downregulation or loss of detectable antigen on tumor cells), leading to tumor recurrence after treatment. For example, in a Phase 1/2 clinical study (ZUMA-1) of axicabtagene ciloleucel, an autologous anti-CD19 CAR T-cell product, 39 of 88 (44%) responders later relapsed after treatment and, among all patients with available post-relapse samples, 4 of 16 (25%) patients presented with CD19-positive disease at diagnosis and CD19-negative disease after treatment. See, e.g., Locke et al., (2019) Lancet Oncol. 2019(1):31-42, and Neelapu et al., (2017) The New England journal of medicine 2017; 377 (26):2531-44, which are incorporated herein by reference for any purpose in their entireties. Additionally, patients receiving tisagenlecleucel, another anti-CD19 CAR T-cell therapy, suffered from disease relapse driven by antigen loss. See, e.g., Maude et al., (2018) The New England Journal of Medicine 2018; 378 (5):439-48, and Maude et al., (2016) Journal of Clinical Oncology 2016; 34 (15_suppl):3011, which are incorporated herein by reference for any purpose in their entireties. Nonclinical data have demonstrated that a dual-targeting approach, with CARs directed towards 2 independent target cell-surface antigens, is more efficacious in vitro and in vivo compared with monovalent CAR T cells See, e.g., Hegde et al., (2013) Mol. Ther. 2013; 21 (11):2087-101, Hegde et al., (2016) J. Clin. Invest 2016; 126 (8):3036-52, Ruella et al., (2018) Mol. Ther Oncolytics. 2018; 11:127-37, and Zah et al., (2016) J. Clin. Invest Cancer Immunol. Res. 2016; 4 (6):498-508, which are incorporated herein by reference for any purpose in their entireties. Similar to CD19, CD20 is a cell-surface antigen expressed in most healthy B cells, from pre-B cell to memory B-cell stages, as well as leukemia and lymphoma cells. Proof of concept of targeting CD20 has been demonstrated in the clinic in the context of both monoclonal antibody therapies and CAR T-cell therapies, and when combined with CD19 targeting, could represent an effective strategy to reduce the probability of antigen escape. See, e.g., Boye et al., (2003) Annals of Oncology 2003; 14 (4):520-35, Brudno et al., (2018) Nat. Rev. Clin. Oncol. 2018; 15 (1):31-46, and Zah et al., (2016) J. Clin. Invest Cancer Immunol. Res. 2016; 4 (6):498-508, which are incorporated herein by reference for any purpose in their entireties.

Both single and dual antigens such as those targeting anti-CD19/CD20 CAR T-cell therapy, for the treatment of patients with relapsed or refractory B-cell malignancies need to be well understood and characterized during the development and manufacturing process. More specifically, the embodiments herein describe antibodies specific for scFv14 in order to characterize the specific protein expression of anti-CD20 CARs.

There is a further need for the detection and quantification of viral particles. In certain aspects, detection of viral particles can be achieved by detection of viral envelope proteins such as gibbon ape leukemia virus (GALV) protein gp70. Antigen binding molecules specific for GALV gp70, as disclosed herein, have many uses, for example in assays such as flow based viral detection methods.

### III. Antigen Binding Molecules: Specific to Anti-CD20 scFv14

The present disclosure is directed to antigen binding molecules, including antibodies, that specifically bind the anti-CD20 scFv14, as well as molecules comprising the same sequence and cells presenting such molecules, and/or those which cross compete with one or more antigen binding molecules described herein. Heavy chain antigen binding molecules may comprise a set of unique CDR sequences as defined in Tables 3A, 3B, and 3C and are exemplified by the provided light chain CDR1, CDR2, and CDR3 sequences set forth in Tables 4A, 4B, and 4C. Related clones may be found in Tables 5 and 6. In various embodiments, a scFv form of the antigen binding molecule may include a heavy chain binding molecule be joined to a light chain binding molecule by a linker amino acid sequence (e.g., a “Whitlow” linker). Examples of linker sequences are described herein.

In various embodiments, an antigen binding molecule described herein may be used in one of more methods (e.g., the methods described herein and in the art).

In various embodiments, an antigen binding molecule may comprise one or more CDRs. In various embodiments, an antigen binding molecule may comprise one or more framework regions. In various embodiments, an antigen binding mole may comprise three CDRs spaced apart and between four framework regions.

In various embodiments, an antigen binding molecule may comprise one or more CDRs incorporated into a variable heavy chain. In various embodiments, an antigen binding molecule may comprise one or more CDRs incorporated into a variable light chain. In various embodiments, an antigen binding molecule may comprise a variable heavy chain connected to a variable light chain by a linker.

In various embodiments, an antigen binding molecule may comprise a light chain. In various embodiments, an antigen binding molecule may comprise a heavy chain. In various embodiments, an antigen binding molecule may comprise a light chain and a heavy chain connected by a disulfide linkage. In various embodiments, an antigen binding molecule may comprise a first heavy chain connected to a second heavy chain by a disulfide linkage. In various embodiments, an antigen binding molecule may comprise two light chains and two heavy chains.

An antibody or antigen binding molecule encoded of the present disclosure can be single chained or double chained. In some embodiments, the antibody or antigen binding molecule may be single chained. In certain embodiments, the antigen binding molecule may be selected from the group consisting of an scFv, a Fab, a Fab′, a Fv, a F(ab′)₂, a dAb, and any combination thereof. In one particular embodiment, the antibody or antigen binding molecule may comprise a a scFv.

In certain embodiments, an antigen binding molecule such as an antibody may comprise a single chain, wherein the heavy chain variable region and the light chain variable region may be connected by a linker. In some embodiments, the V_{H} may be located at the N terminus of the linker and the V_{L} may be located at the C terminus of the linker. In other embodiments, the V_{L} may be located at the N terminus of the linker and the V_{H} may be located at the C terminus of the linker.

TABLE 3A
Anti-CD20 heavy chain antigen binding molecule CDR1, CDR2, and CDR3
amino acid sequences (Chothia Format).

| Clone ID # | SEQ_aa_CDR1 | SEQ_aa_CDR2 | SEQ_aa_CDR3 |
|---|---|---|---|
| 22H5 | GYSITSGY | SYSGS (SEQ ID NO: | EGYSNYFDS (SEQ |
| | (SEQ ID NO: | 42) | ID NO: 66) |
| | 21) | | |
| 23C11 | GYTFTSY | DPSDSE (SEQ ID NO: | AGRVFYYAMDY |
| | (SEQ ID NO: | 43) | (SEQ ID NO: 67) |
| | 22) | | |
| 23E1 | GFTFTDY--- | -----YPYNGG--------- | RGQRVWYFDV |
| | (SEQ ID NO: | (SEQ ID NO: 44) | (SEQ ID NO: 68) |
| | 23) | | |
| 24C7 | GYSFTSY (SEQ | YPGSGN (SEQ ID NO: | CVLLDYFDY (SEQ |
| | ID NO: 24) | 45) | ID NO: 69) |
| 24C12 | GHTFTGY | LPGSGS (SEQ ID NO: | EGFAY (SEQ ID NO: |
| | (SEQ ID NO: | 46) | 70) |
| | 25) | | |
| 29F8 | GYTFTTY | NTYSGV (SEQ ID NO: | YYYGSNYDY (SEQ |
| | (SEQ ID NO: | 47) | ID NO: 71) |
| | 26) | | |
| 25B2 | GYTFTTY | NTYSGV (SEQ ID NO: | TYYGNYFDY (SEQ |
| | (SEQ ID NO: | 47) | ID NO: 72) |
| | 26) | | |
| 29F1 | GYTFTTY | NTYSGV (SEQ ID NO: | FYYGSSFDY (SEQ |
| | (SEQ ID NO: | 47) | ID NO: 73) |
| | 26) | | |
| 25F10 | GYSFTSY (SEQ | YPRSGN (SEQ ID NO: | SDFYYGSDY (SEQ |
| | ID NO: 24) | 48) | ID NO: 74) |
| 18D7 | GFTFSNY (SEQ | RLKSDNYA (SEQ ID | GYYGSRRGFDY |
| | ID NO: 27) | NO: 49) | (SEQ ID NO: 75) |

TABLE 3B
Anti-CD20 heavy chain antigen binding molecule CDR1, CDR2, and CDR3
amino acid sequences (Kabat Format).

| Clone ID # | SEQ_aa_CDR1 | SEQ_aa_CDR2 | SEQ_aa_CDR3 |
|---|---|---|---|
| 22H5 | SGYDWH (SEQ | YISYSGSTNYNPSLKS (SEQ | EGYSNYFDS (SEQ |
| | ID NO: 28) | ID NO: 50) | ID NO: 66) |
| 23C11 | SYWMH (SEQ | NIDPSDSETHYNQKFKD | AGRVFYYAMDY |
| | ID NO: 29) | (SEQ ID NO: 51) | (SEQ ID NO: 67) |
| 23E1 | -----DYYMH | ---LVYPYNGGTSYNQKFKG | RGQRVWYFDV |
| | (SEQ ID NO: | (SEQ ID NO: 52) | (SEQ ID NO: 68) |
| | 30) | | |
| 24C7 | SYYIH (SEQ ID | WIYPGSGNTKYNEKFKG | CVLLDYFDY (SEQ |
| | NO: 31) | (SEQ ID NO: 53) | ID NO: 69) |
| 24C12 | GYWIE (SEQ | EILPGSGSTNYNEKFKG | EGFAY (SEQ ID |
| | ID NO: 32) | (SEQ ID NO: 54) | NO: 70) |
| 29F8 | TYGMS (SEQ | WINTYSGVPTYADDFKG | YYYGSNYDY |
| | ID NO: 33) | (SEQ ID NO: 55) | (SEQ ID NO: 71) |
| 25B2 | TYGMS (SEQ | WINTYSGVPTYADDFKG | TYYGNYFDY (SEQ |
| | ID NO: 33) | (SEQ ID NO: 55) | ID NO: 72) |
| 29F1 | TYGMS (SEQ | WINTYSGVPTYADDFKG | FYYGSSFDY (SEQ |
| | ID NO: 33) | (SEQ ID NO: 55) | ID NO: 73) |
| 25F10 | SYYIH (SEQ ID | WIYPRSGNTNYNEKFKD | SDFYYGSDY (SEQ |
| | NO: 31) | (SEQ ID NO: 56) | ID NO: 74) |
| 18D7 | NYWMN (SEQ | QIRLKSDNYATHYAESVKG | GYYGSRRGFDY |
| | ID NO: 34) | (SEQ ID NO: 57) | (SEQ ID NO: 75) |

TABLE 3C
Anti-CD20 heavy chain antigen binding molecule CDR1, CDR2, and CDR3
amino acid sequences (IMGT Format).

| Clone ID # | SEQ_aa_CDR1 | SEQ_aa_CDR2 | SEQ_aa_CDR3 |
|---|---|---|---|
| 22H5 | GYSITSGYD | ISYSGST (SEQ ID NO: 58) | AREGYSNYFDS |
| | (SEQ ID NO: | | (SEQ ID NO: 76) |
| | 35) | | |
| 23C11 | GYTFTSYW | IDPSDSET (SEQ ID NO: 59) | ARAGRVFYYAMDY |
| | (SEQ ID NO: | | (SEQ ID NO: 77) |
| | 36) | | |
| 23E1 | GFTF-Y-- | ----VYPYNGGT-------- (SEQ | ARRGQRVWYFDV |
| | (SEQ ID NO: | ID NO: 60) | (SEQ ID NO: 78) |
| | 37) | | |
| 24C7 | GYSFTSYY | IYPGSGNT (SEQ ID NO: | ARCVLLDYFDY |
| | (SEQ ID NO: | 61) | (SEQ ID NO: 79) |
| | 38) | | |
| 24C12 | GHTFTGYW | ILPGSGST (SEQ ID NO: 62) | AREGFAY (SEQ ID |
| | (SEQ ID NO: | | NO: 80) |
| | 39 | | |
| 29F8 | GYTFTTYG | INTYSGVP (SEQ ID NO: | ARYYYGSNYDY |
| | (SEQ ID NO: | 63) | (SEQ ID NO: 81) |
| | 40) | | |
| 25B2 | GYTFTTYG | INTYSGVP (SEQ ID NO: | ARTYYGNYFDY |
| | (SEQ ID NO: | 63) | (SEQ ID NO: 82) |
| | 40) | | |
| 29F1 | GYTFTTYG | INTYSGVP (SEQ ID NO: | ARFYYGSSFDY |
| | (SEQ ID NO: | 63) | (SEQ ID NO: 83) |
| | 40) | | |
| 25F10 | GYSFTSYY | IYPRSGNT (SEQ ID NO: | GRSDFYYGSDY |
| | (SEQ ID NO: | 64) | (SEQ ID NO: 84) |
| | 38) | | |
| 18D7 | GFTFSNYW | IRLKSDNYAT (SEQ ID NO: | TGGYYGSRRGFDY |
| | (SEQ ID NO: | 65) | (SEQ ID NO: 85) |
| | 41) | | |

TABLE 4A
Anti-CD20 light chain antigen binding molecule CDR1, CDR2, and CDR3
sequences (Chothia Format).

| SEQ_aa_CDR1 | SEQ_aa_CDR2 | SEQ_aa_CDR3 |
|---|---|---|
| KASQSVSNDVA (SEQ ID | YASNRYT (SEQ ID NO: | QQDYSSPLA (SEQ ID NO: |
| NO: 86) | 100) | 112) |
| RASQDISNYLN (SEQ ID | YTSRLHS (SEQ ID NO: | QQGNTLYT (SEQ ID NO: |
| NO: 87) | 101) | 113) |
| KASQDINKYIA (SEQ ID | YTSTLQP (SEQ ID NO: | LQYDNLYT (SEQ ID NO: |
| NO: 88) | 102) | 114) |
| RASQEISGYLS (SEQ ID | AASTLDS (SEQ ID NO: | LQYASYPYT (SEQ ID NO: |
| NO: 89) | 103) | 115) |
| KASQDVGIAVA (SEQ ID | WASTRHT (SEQ ID NO: | QQYSSYPYT (SEQ ID NO: |
| NO: 90) | 104) | 116) |
| RASENIYSYLA (SEQ ID | NAKTLAE (SEQ ID NO: | QHHYGSPPT (SEQ ID NO: |
| NO: 91) | 105) | 117) |
| KASQDINKYIA (SEQ ID | YTSTLQP (SEQ ID NO: | LQYDNLYT (SEQ ID NO: |
| NO: 88) | 102) | 114) |
| RASENIYSYLA (SEQ ID | NAKTLAE (SEQ ID NO: | QHHYGTPLT (SEQ ID NO: |
| NO: 91) | 105) | 118) |
| RASQEISGYLG (SEQ ID | AASTLDS (SEQ ID NO: | LQYASYPWT (SEQ ID NO: |
| NO: 92) | 103) | 119) |
| SASQGISNYLN (SEQ ID | YTSSLHS (SEQ ID NO: | QQYSKLPFT (SEQ ID NO: |
| NO: 93) | 106) | 120) |

TABLE 4B
Anti-CD20 light chain antigen binding molecule CDR1, CDR2, and CDR3
sequences (Kabat Format).

| SEQ_aa_CDR1 | SEQ_aa_CDR2 | SEQ_aa_CDR3 |
|---|---|---|
| KASQSVSNDVA (SEQ ID | YASNRYT (SEQ ID NO: | QQDYSSPLA (SEQ ID NO: |
| NO: 86) | 100) | 112) |
| RASQDISNYLN (SEQ ID | YTSRLHS (SEQ ID NO: | QQGNTLYT (SEQ ID NO: |
| NO: 87) | 101) | 113) |
| KASQDINKYIA (SEQ ID | YTSTLQP (SEQ ID NO: | LQYDNLYT (SEQ ID NO: |
| NO: 88) | 102) | 114) |
| RASQEISGYLS (SEQ ID | AASTLDS (SEQ ID NO: | LQYASYPYT (SEQ ID NO: |
| NO: 89) | 103) | 115) |
| KASQDVGIAVA (SEQ ID | WASTRHT (SEQ ID NO: | QQYSSYPYT (SEQ ID NO: |
| NO: 90) | 104) | 116) |
| RASENIYSYLA (SEQ ID | NAKTLAE (SEQ ID NO: | QHHYGSPPT (SEQ ID NO: |
| NO: 91) | 105) | 117) |
| KASQDINKYIA (SEQ ID | YTSTLQP (SEQ ID NO: | LQYDNLYT (SEQ ID NO: |
| NO: 88) | 102) | 114) |
| RASENIYSYLA (SEQ ID | NAKTLAE (SEQ ID NO: | QHHYGTPLT (SEQ ID NO: |
| NO: 91) | 105) | 118) |
| RASQEISGYLG (SEQ ID | AASTLDS (SEQ ID NO: | LQYASYPWT (SEQ ID NO: |
| NO: 92) | 103) | 119) |
| SASQGISNYLN (SEQ ID | YTSSLHS (SEQ ID NO: | QQYSKLPFT (SEQ ID NO: |
| NO: 93) | 106) | 120) |

TABLE 4C
Anti-CD20 light chain antigen binding molecule CDR1, CDR2, and CDR3
sequences (IMGT Format).

| SEQ_aa_CDR1 | SEQ_aa_CDR2 | SEQ_aa_CDR3 |
|---|---|---|
| QSVSND (SEQ ID NO: 94) | YAS | QQDYSSPLA (SEQ ID NO: |
| | | 112) |
| QDISNY (SEQ ID NO: 95) | YTS | QQGNTLYT (SEQ ID NO: |
| | | 113) |
| QDINKY (SEQ ID NO: 96) | YTS | LQYDNLYT (SEQ ID NO: |
| | | 114) |
| QEISGY (SEQ ID NO: 97) | AAS | LQYASYPYT (SEQ ID NO: |
| | | 115) |
| QDVGIA (SEQ ID NO: 98) | WAS | QQYSSYPYT (SEQ ID NO: |
| | | 116) |
| ENIYSY (SEQ ID NO: 99) | NAK | QHHYGSPPT (SEQ ID NO: |
| | | 117) |
| QDINKY (SEQ ID NO: 96) | YTS | LQYDNLYT (SEQ ID NO: |
| | | 114) |
| ENIYSY (SEQ ID NO: 99) | NAK | QHHYGTPLT (SEQ ID NO: |
| | | 118) |
| QEISGY (SEQ ID NO: 97) | AAS | LQYASYPWT (SEQ ID NO: |
| | | 119) |
| QDISNY (SEQ ID NO: 95) | YTS | QQYSKLPFT (SEQ ID NO: |
| | | 120) |

TABLE 5
Anti-CD20 heavy chain antigen binding molecule variable domain amino acid
sequences.

| Clone | | SEQ ID |
|---|---|---|
| ID # | SEQ_aa_FR1_FR4 | NO. |
| 22H5 | DVQLQESGPGMVKPSQSLSLTCTVTGYSITSGYDWHWIRHFPGN | 1 |
| | KLEWMGYISYSGSTNYNPSLKSRISITHDTSKNHFFLKLNSVTTED | |
| | TATYYCAREGYSNYFDSWGQGTTLTVSS | |
| 23C11 | QVQLQQPGAELVRPGSSVKLSCKASGYTFTSYWMHWVKQRPIQ | 2 |
| | GLEWIGNIDPSDSETHYNQKFKDKATLTVDKSSSTAYMQLSSLTS | |
| | EDSAVYYCARAGRVFYYAMDYWGQGTSVTVSS | |
| 23E1 | EVQLQQSGPVLVKPGPSVKISCKASGFTFTDYYMHWVKQSHGKS | 3 |
| | LEWIGLVYPYNGGTSYNQKFKGKATLTVDTSSSTAYMELNSLTS | |
| | EDSAVYYCARRGQRVWYFDVWGTGTTVTVSS | |
| 24C7 | QVQLQQSGPELVKPGASVKISCKASGYSFTSYYIHWVKQRPGQG | 4 |
| | LEWIGWIYPGSGNTKYNEKFKGKATLTADTSSSTAYMQLSSLTSE | |
| | DSAVYYCARCVLLDYFDYWGQGTTLTVSS | |
| 24C12 | QVQLQQSGAELMKPGASVKLSCKATGHTFTGYWIEWVKQRPGH | 5 |
| | GLEWIGEILPGSGSTNYNEKFKGKATFTADTSSNTAYMQLSSLTT | |
| | EDSAIYYCAREGFAYWGQGTLVTVSA | |
| 29F8 | QIQLVQSGPELKKPGETVKISCKASGYTFTTYGMSWVKQAPGKG | 6 |
| | LKWMGWINTYSGVPTYADDFKGRFAFSLETSASTAYLQINNLKN | |
| | EDSATYFCARYYYGSNYDYWGQGTTLTVSS | |
| 25B2 | QIQLVQSGPELKKPGETVKISCKASGYTFTTYGMSWVKQAPGKG | 7 |
| | LKWMGWINTYSGVPTYADDFKGRFAFSLETSASTAYLQINNLKN | |
| | EDTATYFCARTYYGNYFDYWGQGTTLTVSS | |
| 29F1 | QIQLVQSGPELKKPGETVKISCKASGYTFTTYGMSWVKQAPGKG | 8 |
| | LKWMGWINTYSGVPTYADDFKGRFAFSLETSASTAYLQINNLKN | |
| | EDTATYFCARFYYGSSFDYWGQGTTLTVSS | |
| 25F10 | QVHLQQSGPELVKPGASVKISCKASGYSFTSYYIHWVKQRPGQG | 9 |
| | LEWIGWIYPRSGNTNYNEKFKDKATLAADTSSSAAYMQLSSLTS | |
| | EDSAVYYCGRSDFYYGSDYWGQGTTLTVSS | |
| 18D7 | EVKLEESGGGLVQPGGSMKLSCVASGFTFSNYWMNWVRQSPEK | 10 |
| | GLEWVAQIRLKSDNYATHYAESVKGRFTISRDDSKSSVYLQMNN | |
| | LRAEDTGIYYCTGGYYGSRRGFDYWGQGTTLTVSS | |

TABLE 6
Anti-CD20 light chain antigen binding molecule variable domain amino acid
sequences.

| Clone | | SEQ ID |
|---|---|---|
| ID # | SEQ_aa_FR1_FR4 | NO. |
| 22H5 | SIVMTQTPKFLLVSAGDRVTITCKASQSVSNDVAWYQQKPGQSP | 11 |
| | KLLIFYASNRYTGVPDRFTGSGYGTDFTFTISTVQAEDLAVYFCQ | |
| | QDYSSPLAFGAGTKLELK | |
| 23C11 | DIQMTQTTSSLSASLGDRVTISCRASQDISNYLNWYQQKPDGTVK | 12 |
| | LLIYYTSRLHSGVPSRFSGSGSGTDYSLTISNLEQEDIATYFCQQG | |
| | NTLYTFGGGTKLEIK | |
| 23E1 | DIQMTQSPSSLSASLGGKVTITCKASQDINKYIAWYQHKPGKGPR | 13 |
| | LLIHYTSTLQPGIPSRFSGSGSGRDYSFSISNLEPEDIATYYCLQYD | |
| | NLYTFGGGTKLEIK | |
| 24C7 | DIQMTQSPSSLSASLGERVSLTCRASQEISGYLSWLQQKPDGTIKR | 14 |
| | LIYAASTLDSGVPKRFSGSRSGSDYSLTISSLESEDFADYYCLQYA | |
| | SYPYTFGGGTKLEIK | |
| 24C12 | DIVMTQSHKFMSTSVGDRVSITCKASQDVGIAVAWYQQKPGQSP | 15 |
| | KLLIYWASTRHTGVPDRFTGSGSGTDFTLTISNVQSEDLADYFCQ | |
| | QYSSYPYTFGGGTKLEIK | |
| 29F8 | DIQMTQSPASLSASVGETVTITCRASENIYSYLAWYQQKQGKSPQ | 16 |
| | LLVNNAKTLAEGVPSRFSGSGSGTQFSLKINSLQPEDFGSYYCQH | |
| | HYGSPPTFGSGTKLEIK | |
| 25B2 | DIQMTQSPSSLSASLGGKVTITCKASQDINKYIAWYQHKPGKGPR | 17 |
| | LLIHYTSTLQPGIPSRFSGSGSGRDYSFSISNLEPEDIATYYCLQYD | |
| | NLYTFGGGTKLEIK | |
| 29F1 | DIQMTQSPASLSASVGETVTITCRASENIYSYLAWYQQKQGKSPQ | 18 |
| | LLVYNAKTLAEGVPSRFSGSGSGTQFSLKINSLQPEDFGSYYCQH | |
| | HYGTPLTFGAGTKLELK | |
| 25F10 | DIQMTQSPSSLSASLGERVSLTCRASQEISGYLGWLQQKPDGTIKR | 19 |
| | LIYAASTLDSGVPRRFSGSRSGSDYSLTFSSLESEDFADYYCLQYA | |
| | SYPWTFGGGTKLEIK | |
| 18D7 | DIQMTQTTSSLSASLGDRVTISCSASQGISNYLNWYQQKPDGTVK | 20 |
| | LLIYYTSSLHSGVPSRESGSGSGTDYSLTISNLEPEDIATYYCQQYS | |
| | KLPFTFGSGTKLEIK | |

In one embodiment, the antigen binding molecules of the present disclosure are antibodies and antigen binding fragments thereof. In one embodiment, the antibodies specific to anti-CD20 scFv of the present disclosure comprise at least one CDR set forth in Tables 3A-3C and 4A-4C. In another aspect, the present disclosure provides hybridomas capable of producing the antibodies disclosed herein and methods of producing antibodies from hybridomas, as described herein and as known in the art.

Humanized antibodies are described herein and may be prepared by known techniques. In one embodiment, a humanized monoclonal antibody comprises the variable domain of a murine or rabbit antibody (or all or part of the antigen binding site thereof) and a constant domain derived from a human antibody. Alternatively, a humanized antibody fragment may comprise an antigen binding site of a murine or rabbit monoclonal antibody and a variable domain fragment (lacking the antigen binding site) derived from a human antibody. Procedures for the production of engineered monoclonal antibodies include those described in Riechmann et al., (1988) Nature 332:323, Liu et al., (1987) Proc. Nat. Acad. Sci. USA 84:3439, Larrick et al., (1989) Bio/Technology 7:934, and Winter et al., (1993) TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557; Padlan et al., (1995) FASEB J. 9:133-39; Tamura et al., (2000) J. Immunol. 164:1432-41; Zhang et al., (2005) Mol. Immunol. 42(12):1445-1451; Hwang et al., Methods. (2005) 36(1):35-42; Dall'Acqua et al., (2005) Methods 36(1):43-60; and Clark, (2000) Immunology Today 21(8):397-402.

An antigen binding molecule of the present invention can also be a fully human monoclonal antibody. Fully human monoclonal antibodies can be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., (1997) Curr. Opin. Biotechnol. 8:455-58).

Examples of techniques for production and use of transgenic animals for the production of human or partially human antibodies are described in U.S. Pat. Nos. 5,814,318, 5,569,825, and 5,545,806; Davis et al., Antibody Engineering: Methods and Protocols, (Lo, ed) Humana Press, NJ, 191-200 (2003); Kellermann et al., (2002) Curr Opin Biotechnol. 13:593-97; Russel et al., (2000) Infect Immun. 68:1820-26; Gallo et al., (2000) Eur J. Immun. 30:534-40; Davis et al., (1999) Cancer Metastasis Rev. 18:421-25; Green, (1999) J Immunol Methods 231:11-23; Jakobovits, (1998) Advanced Drug Delivery Reviews 31:33-42; Green et al., (1998) J Exp Med. 188:483-95; Jakobovits, (1998) Exp. Opin. Invest. Drugs. 7:607-14; Tsuda et al., (1997) Genomics, 42:413-21; Mendez et al., (1997) Nat. Genet. 15:146-56; Jakobovits, (1994) Curr Biol. 4:761-63; Arbones et al., (1994) Immunity 1:247-60; Green et al., (1994) Nat. Genet. 7:13-21; Jakobovits et al., (1993) Nature 362:255-58; Jakobovits et al., (1993) Proc Natl Acad Sci USA 90:2551-55; Chen et al., (1993) Intl Immunol 5:647-656; Choi et al., (1993) Nature Genetics 4:117-23; Fishwild et al., (1996) Nature Biotechnology 14:845-51; Lonberg et al., (1994) Nature 368: 856-59; Lonberg, (1994) Handbook of Experimental Pharmacology 113: 49-101; Neuberger, (1996) Nature Biotech 14:826; Taylor et al., (1992) Nucleic Acids Research 20:6287-95; Taylor et al., (1994) Intl Immunol 6:579-91; Tomizuka et al., (1997) Nature Genetics 16:133-43; Tomizuka et al., (2000) Proc Nat Acad Sci USA 97:722-27; Tuaillon et al., (1993) Proc Nat Acad Sci USA 90:3720-24; Tuaillon et al., (1994) J Immunol 152:2912-20.; Lonberg et al., (1994) Nature 368:856; Taylor et al., (1994) Intl Immunol 6:579; U.S. Pat. No. 5,877,397; Bruggemann et al., (1997) Curr. Opin. Biotechnol. 8:455-58; Jakobovits et al., (1995) Ann. N. Y. Acad. Sci. 764:525-35.

An additional method for obtaining antigen binding molecules of the invention is by the use of phage display, which is well-established for this purpose. See, e.g., Winter et al., (1994) Ann. Rev. Immunol. 12:433-55; Burton et al., (1994) Adv. Immunol 57:191-280. Human or murine immunoglobulin variable region gene combinatorial libraries can be created in phage vectors that can be screened to select Ig fragments (Fab, Fv, sFv, or multimers thereof) that bind the scFv-14, as well as molecules comprising this sequence and cells presenting such molecules. See, e.g., U.S. Pat. No. 5,223,409; Huse et al., (1989) Science 246:1275-81; Sastry et al., (1989) Proc. Natl. Acad. Sci. USA 86:5728-32; Alting-Mees et al., (1990) Strategies in Molecular Biology 3:1-9; Kang et al., (1991) Proc. Natl. Acad. Sci. USA 88:4363-66; Hoogenboom et al., (1992) J. Mol. Biol. 227:381-388; Schlebusch et al., (1997) Hybridoma 16:47-52 and references cited therein. For example, a library containing a plurality of polynucleotide sequences encoding Ig variable region fragments can be inserted into the genome of a filamentous bacteriophage, such as M13 or lambda phage (λImmunoZap™(H) and λImmunoZap™(L) vectors (Stratagene, La Jolla, Calif) can also be used in this approach) or a variant thereof, in frame with the sequence encoding a phage coat protein.

Briefly, mRNA is isolated from a B-cell population, and used to create heavy and light chain immunoglobulin cDNA expression libraries in the λImmunoZap™(H) and λImmunoZap™(L) and similar vectors. These vectors can be screened individually or co-expressed to form Fab fragments or antibodies. Positive plaques can subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers can be synthesized by one of ordinary skill in the art, or can be purchased from commercial sources, which also sell primers for mouse and human variable regions including, among others, primers for V_{H}, V_{L}, C_{H} and C_{L} regions). These primers can be used to amplify heavy or light chain variable regions, which can then be inserted into vectors. These vectors can then be introduced into E. coli, yeast, or mammalian-based systems for expression. Large amounts of a single-chain protein containing a fusion of the V_{H} and V_{L} domains can be produced using these methods.

Once cells producing the antigen binding molecules provided herein have been obtained using any of the above-described immunization and other techniques, the specific antibody genes can be cloned by isolating and amplifying DNA or mRNA therefrom according to standard procedures as described herein. The antibodies produced therefrom can be sequenced and the CDRs identified and the DNA coding for the CDRs can be manipulated as described previously to generate other antibodies according to the invention.

It will be understood by those of skill in the art that some proteins, such as antibodies, can undergo a variety of posttranslational modifications. The type and extent of these modifications often depends on the host cell line used to express the protein as well as the culture conditions. Such modifications can include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in, e.g., Harris, (1995) J Chromatog 705:129-34).

An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, e.g., Baines and Thorpe, (1992) in Methods in Molecular Biology, 10:79-104 (The Humana Press). Monoclonal antibodies can be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anti-constant region (light chain or heavy chain) antibody, and an anti-idiotype antibody.

Although the disclosed antigen binding molecules were produced in a mouse system, human, partially human, or humanized antibodies may be suitable for many applications, particularly those involving administration of the antibody to a human subject, other types of antigen binding molecules will be suitable for certain applications. Such antibodies can be prepared as described herein and form an aspect of the instant disclosure.

The instant disclosure provides antigen binding molecules that specifically bind to anti-CD20 scFv-14 binding domain and subsequences thereof, molecules comprising this sequence and cells presenting such molecules. Antigen binding molecules that cross compete with the antigen binding molecules disclosed herein form another aspect of the instant disclosure.

In some embodiments, the antibody or antigen binding molecule that specifically binds anti-CD20 scFv-14 binding domain binds the same or an overlapping epitope as a reference antibody disclosed herein. In certain embodiments, the antibody or antigen binding molecule binds the same or an overlapping epitope as a reference antibody.

### IV. Antigen Binding Molecules: Specific to GALV Gp70 Protein

The present disclosure is further directed to antigen binding molecules, including antibodies, that specifically bind GALV gp70 protein, as well as molecules comprising the same sequence and cells presenting such molecules, and/or those which cross compete with one or more antigen binding molecules described herein. Heavy chain antigen binding molecules may comprise a set of unique CDR sequences as defined in Tables 7A, 7B, and 7C and are exemplified by the provided light chain CDR1, CDR2, and CDR3 sequences set forth in Tables 8A, 8B, and 8C. Related clones may be found in Tables 9 and 10. In various embodiments, a scFv form of the antigen binding molecule may include a heavy chain binding molecule joined to a light chain binding molecule by a linker amino acid sequence (e.g., a “Whitlow” linker). Examples of linker sequences are described herein.

In various embodiments, an antigen binding molecule described herein may be used in one of more methods (e.g., the methods described herein and in the art).

In various embodiments, an antigen binding molecule may comprise one or more CDRs. In various embodiments, an antigen binding molecule may comprise one or more framework regions. In various embodiments, an antigen binding mole may comprise three CDRs spaced apart and between four framework regions.

In various embodiments, an antigen binding molecule may comprise one or more CDRs incorporated into a variable heavy chain. In various embodiments, an antigen binding molecule may comprise one or more CDRs incorporated into a variable light chain. In various embodiments, an antigen binding molecule may comprise a variable heavy chain connected to a variable light chain by a linker.

In various embodiments, an antigen binding molecule may comprise a light chain. In various embodiments, an antigen binding molecule may comprise a heavy chain. In various embodiments, an antigen binding molecule may comprise a light chain and a heavy chain connected by a disulfide linkage. In various embodiments, an antigen binding molecule may comprise a first heavy chain connected to a second heavy chain by a disulfide linkage. In various embodiments, an antigen binding molecule may comprise two light chains and two heavy chains.

An antibody or antigen binding molecule encoded of the present disclosure can be single chained or double chained. In some embodiments, the antibody or antigen binding molecule may be single chained. In certain embodiments, the antigen binding molecule may be selected from the group consisting of an scFv, a Fab, a Fab′, a Fv, a F(ab′)₂, a dAb, and any combination thereof. In one particular embodiment, the antibody or antigen binding molecule may comprise a scFv.

In certain embodiments, an antigen binding molecule such as an antibody may comprise a single chain, wherein the heavy chain variable region and the light chain variable region may be connected by a linker. In some embodiments, the V_{H} may be located at the N terminus of the linker and the V_{L} may be located at the C terminus of the linker. In other embodiments, the V_{L} may be located at the N terminus of the linker and the V_{H} may be located at the C terminus of the linker.

TABLE 7A
GALV gp70 heavy chain antigen binding molecule CDR1, CDR2, and CDR3
amino acid sequences (Chothia Format).

| Clone ID # | SEQ_aa_CDRH1 | SEQ_aa_CDRH2 | SEQ_aa_CDRH3 |
|---|---|---|---|
| 9G11 | GYTFTNY | YPSDSE | DYY |
| | (SEQ ID NO: 127) | (SEQ ID NO: 139) | |
| 40A6 | GFTFSSY | NSGGSY | HLIYYDYDGYYFDT |
| | (SEQ ID NO: 128) | (SEQ ID NO: 140) | (SEQ ID NO: 151) |
| 40A3 | DFSITSDY | HYSGG | YGSRDAVDY |
| | (SEQ ID NO: 129) | (SEQ ID NO: 141) | (SEQ ID NO: 152) |
| 3C8 V2 | GYTFTHY | YPSDGE | DYY |
| | (SEQ ID NO: 130) | (SEQ ID NO: 142) | |
| 3C8 V1 | GYTFTDY | TPNKGD | GKNYSGSSLHWYFDV |
| | (SEQ ID NO: 131) | (SEQ ID NO: 143) | (SEQ ID NO: 153) |
| 36H3 | GFTFSNY | SYSGAY | HVEYYDYDPYALDF |
| | (SEQ ID NO: 132) | (SEQ ID NO: 144) | (SEQ ID NO: 154) |
| 35C11 | GYTFTDY | STYSGK | GGLRDAMDY |
| | (SEQ ID NO: 133) | (SEQ ID NO: 145) | (SEQ ID NO: 155) |
| 2D3 | GYTFTNY | YPSDSE | DYY |
| | (SEQ ID NO: 134) | (SEQ ID NO: 146) | |
| 13E7 | GYTFTSH | DPNSGG | EGYEGYLGYFDF |
| | (SEQ ID NO: 135) | (SEQ ID NO: 147) | (SEQ ID NO: 156) |
| 4F1 | GYTFTSY | YPSDSE | DYY |
| | (SEQ ID NO: 136) | (SEQ ID NO: 148) | |
| 9A1 | GYTFTSY | YPSDSE | DYY |
| | (SEQ ID NO: 137) | (SEQ ID NO: 149) | |
| 8G8 | GYTFTNY | YPSDSE | DYY |
| | (SEQ ID NO: 138) | (SEQ ID NO: 150) | |

TABLE 7B
GALV gp70 heavy chain antigen binding molecule CDR1, CDR2, and CDR3
amino acid sequences (Kabat Format).

| Clone ID # | SEQ_aa_CDRH1 | SEQ_aa_CDRH2 | SEQ_aa_CDRH3 |
|---|---|---|---|
| 9G11 | NYWMD | HIYPSDSETHYIQKFKD | DYY |
| | (SEQ ID NO: 157) | (SEQ ID NO: 169) | |
| 40A6 | SYVLS | TINSGGSYTYYPDSVKG | HLIYYDYDGYYFDT |
| | (SEQ ID NO: 158) | (SEQ ID NO: 170) | (SEQ ID NO: 181) |
| 40A3 | SDYTWH | YIHYSGGTNYNPSLRS | YGSRDAVDY |
| | (SEQ ID NO: 159) | (SEQ ID NO: 171) | (SEQ ID NO: 182) |
| 3C8 V2 | HYWMD | HIYPSDGETHYNQKFRD | DYY |
| | (SEQ ID NO: 160) | (SEQ ID NO: 172) | |
| 3C8 V1 | DYYMN | DITPNKGDTNYNQKFKD | GKNYSGSSLHWYFDV |
| | (SEQ ID NO: 161) | (SEQ ID NO: 173) | (SEQ ID NO: 183) |
| 36H3 | NYGMS | TISYSGAYTYYPDSVKG | HVEYYDYDPYALDF |
| | (SEQ ID NO: 162) | (SEQ ID NO: 174) | (SEQ ID NO: 184) |
| 35C11 | DYTMH | LISTYSGKTNYNQKLKD | GGLRDAMDY |
| | (SEQ ID NO: 163) | (SEQ ID NO: 175) | (SEQ ID NO: 185) |
| 2D3 | NYWMD | NIYPSDSETHYNQKFRD | DYY |
| | (SEQ ID NO: 164) | (SEQ ID NO: 176) | |
| 13E7 | SHWIH | RIDPNSGGTKYNEKFKS | EGYEGYLGYFDF |
| | (SEQ ID NO: 165) | (SEQ ID NO: 177) | (SEQ ID NO: 186) |
| 4F1 | SYWMD | HIYPSDSETHYNQKFKD | DYY |
| | (SEQ ID NO: 166) | (SEQ ID NO: 178) | |
| 9A1 | SYWMD | NIYPSDSETHYNQKFKD | DYY |
| | (SEQ ID NO: 167) | (SEQ ID NO: 179) | |
| 8G8 | NYWMD | NIYPSDSETHYNQNFKD | DYY |
| | (SEQ ID NO: 168) | (SEQ ID NO: 180) | |

TABLE 7C
GALV gp70 heavy chain antigen binding molecule CDR1, CDR2, and CDR3
amino acid sequences (IMGT Format).

| Clone ID # | SEQ_aa_CDRH1 | SEQ_aa_CDRH2 | SEQ_aa_CDRH3 |
|---|---|---|---|
| 9G11 | GYTFTNYW | IYPSDSET | ARDYY |
| | (SEQ ID NO: 187) | (SEQ ID NO: 199) | (SEQ ID NO: 211) |
| 40A6 | GFTFSSYV | INSGGSYT | ARHLIYYDYDGYYFDT |
| | (SEQ ID NO: 188) | (SEQ ID NO: 200) | (SEQ ID NO: 212) |
| 40A3 | DFSITSDYT | IHYSGGT | LYYGSRDAVDY |
| | (SEQ ID NO: 189) | (SEQ ID NO: 201) | (SEQ ID NO: 213) |
| 3C8 V2 | GYTFTHYW | IYPSDGET | ARDYY |
| | (SEQ ID NO: 190) | (SEQ ID NO: 202) | (SEQ ID NO: 214) |
| 3C8 V1 | GYTFTDYY | ITPNKGDT | ARGKNYSGSSLHWYFDV |
| | (SEQ ID NO: 191) | (SEQ ID NO: 203) | (SEQ ID NO: 215) |
| 36H3 | GFTFSNYG | ISYSGAYT | SRHVEYYDYDPYALDF |
| | (SEQ ID NO: 192) | (SEQ ID NO: 204) | (SEQ ID NO: 216) |
| 35C11 | GYTFTDYT | ISTYSGKT | ARGGLRDAMDY |
| | (SEQ ID NO: 193) | (SEQ ID NO: 205) | (SEQ ID NO: 217) |
| 2D3 | GYTFTNYW | IYPSDSET | ARDYY |
| | (SEQ ID NO: 194) | (SEQ ID NO: 206) | (SEQ ID NO: 218) |
| 13E7 | GYTFTSHW | IDPNSGGT | AREGYEGYLGYFDF |
| | (SEQ ID NO: 195) | (SEQ ID NO: 207) | (SEQ ID NO: 219) |
| 4F1 | GYTFTSYW | IYPSDSET | ARDYY |
| | (SEQ ID NO: 196) | (SEQ ID NO: 208) | (SEQ ID NO: 220) |
| 9A1 | GYTFTSYW | IYPSDSET | ARDYY |
| | (SEQ ID NO: 197) | (SEQ ID NO: 209) | (SEQ ID NO: 221) |
| 8G8 | GYTFTNYW | IYPSDSET | ARDYY |
| | (SEQ ID NO: 198) | (SEQ ID NO: 210) | (SEQ ID NO: 222) |

TABLE 8A
GALV gp70 light chain antigen binding molecule CDR1, CDR2, and CDR3
sequences (Chothia Format).

| Clone ID # | SEQ_aa_CDRL1 | SEQ_aa_CDRL2 | SEQ_aa_CDRL3 |
|---|---|---|---|
| 13E7 | RASKNISKYLA | SGSTLQS | QQHYEYPYT |
| | (SEQ ID NO: 223) | (SEQ ID NO: 233) | (SEQ ID NO: 243) |
| 2D3 | RSSTGAVTTSNYAN | GTNNRAL | ALWYSNHLV |
| | (SEQ ID NO: 224) | (SEQ ID NO: 234) | (SEQ ID NO: 244) |
| 35C11 | RASESVDSYGNSFMH | RASNLES | LQSNEDPRT |
| | (SEQ ID NO: 225) | (SEQ ID NO: 235) | (SEQ ID NO: 245) |
| 36H3 | STSSSVTYMH | DTSKLAS | QQWSSSPYT |
| | (SEQ ID NO: 226) | (SEQ ID NO: 236) | (SEQ ID NO: 246) |
| 3C8 | RSNTGAVTTSNYAN | GTNNRVP | ALWYSNHWV |
| | (SEQ ID NO: 227) | (SEQ ID NO: 237) | (SEQ ID NO: 247) |
| 40A6 | RSSQTLVHSNGNIYLH | KVSNRFS | SQTTHVPPT |
| | (SEQ ID NO: 228) | (SEQ ID NO: 238) | (SEQ ID NO: 248) |
| 4F1 | RSSTGAVTTSNYAN | GTNNRAP | ALWYSNHLV |
| | (SEQ ID NO: 229) | (SEQ ID NO: 239) | (SEQ ID NO: 249) |
| 8G8 | RSSTGAVTRSNYAN | GTNNRAL | ALWYSNHLV |
| | (SEQ ID NO: 230) | (SEQ ID NO: 240) | (SEQ ID NO: 250) |
| 9A1 | RSSTGAVTRSNYAN | GTNNRAP | ALWYSNHLV |
| | (SEQ ID NO: 231) | (SEQ ID NO: 241) | (SEQ ID NO: 251) |
| 9G11 | RSSTGAVTTSNYAN | GTNNRAP | ALWYSSQLV |
| | (SEQ ID NO: 232) | (SEQ ID NO: 242) | (SEQ ID NO: 252) |

TABLE 8B
GALV gp70 light chain antigen binding molecule CDR1, CDR2, and CDR3
sequences (Kabat Format).

| Clone ID # | SEQ_aa_CDRL1 | SEQ_aa_CDRL2 | SEQ_aa_CDRL3 |
|---|---|---|---|
| 13E7 | RASKNISKYLA | SGSTLQS | QQHYEYPYT |
| | (SEQ ID NO: 253) | (SEQ ID NO: 263) | (SEQ ID NO: 273) |
| 2D3 | RSSTGAVTTSNYAN | GTNNRAL | ALWYSNHLV |
| | (SEQ ID NO: 254) | (SEQ ID NO: 264) | (SEQ ID NO: 274) |
| 35C11 | RASESVDSYGNSFMH | RASNLES | LQSNEDPRT |
| | (SEQ ID NO: 255) | (SEQ ID NO: 265) | (SEQ ID NO: 275) |
| 36H3 | STSSSVTYMH | DTSKLAS | QQWSSSPYT |
| | (SEQ ID NO: 256) | (SEQ ID NO: 266) | (SEQ ID NO: 276) |
| 3C8 | RSNTGAVTTSNYAN | GTNNRVP | ALWYSNHWV |
| | (SEQ ID NO: 257) | (SEQ ID NO: 267) | (SEQ ID NO: 277) |
| 40A6 | RSSQTLVHSNGNIYLH | KVSNRFS | SQTTHVPPT |
| | (SEQ ID NO: 258) | (SEQ ID NO: 268) | (SEQ ID NO: 278) |
| 4F1 | RSSTGAVTTSNYAN | GTNNRAP | ALWYSNHLV |
| | (SEQ ID NO: 259) | (SEQ ID NO: 269) | (SEQ ID NO: 279) |
| 8G8 | RSSTGAVTRSNYAN | GTNNRAL | ALWYSNHLV |
| | (SEQ ID NO: 260) | (SEQ ID NO: 270) | (SEQ ID NO: 280) |
| 9A1 | RSSTGAVTRSNYAN | GTNNRAP | ALWYSNHLV |
| | (SEQ ID NO: 261) | (SEQ ID NO: 271) | (SEQ ID NO: 281) |
| 9G11 | RSSTGAVTTSNYAN | GTNNRAP | ALWYSSQLV |
| | (SEQ ID NO: 262) | (SEQ ID NO: 272) | (SEQ ID NO: 282) |

TABLE 8C
GALV gp70 light chain antigen binding molecule CDR1, CDR2, and CDR3
sequences (IMGT Format).

| Clone ID # | SEQ_aa_CDRL1 | SEQ_aa_CDRL2 | SEQ_aa_CDRL3 |
|---|---|---|---|
| 13E7 | KNISKY | SGS | QQHYEYPYT |
| | (SEQ ID NO: 283) | | (SEQ ID NO: 293) |
| 2D3 | TGAVTTSNY | GTN | ALWYSNHLV |
| | (SEQ ID NO: 284) | | (SEQ ID NO: 294) |
| 35C11 | ESVDSYGNSF | RAS | LQSNEDPRT |
| | (SEQ ID NO: 285) | | (SEQ ID NO: 295) |
| 36H3 | SSVTY | DTS | QQWSSSPYT |
| | (SEQ ID NO: 286) | | (SEQ ID NO: 296) |
| 3C8 | TGAVTTSNY | GTN | ALWYSNHWV |
| | (SEQ ID NO: 287) | | (SEQ ID NO: 297) |
| 40A6 | QTLVHSNGNIY | KVS | SQTTHVPPT |
| | (SEQ ID NO: 288) | | (SEQ ID NO: 298) |
| 4F1 | TGAVTTSNY | GTN | ALWYSNHLV |
| | (SEQ ID NO: 289) | | (SEQ ID NO: 299) |
| 8G8 | TGAVTRSNY | GTN | ALWYSNHLV |
| | (SEQ ID NO: 290) | | (SEQ ID NO: 300) |
| 9A1 | TGAVTRSNY | GTN | ALWYSNHLV |
| | (SEQ ID NO: 291) | | (SEQ ID NO: 301) |
| 9G11 | TGAVTTSNY | GTN | ALWYSSQLV |
| | (SEQ ID NO: 292) | | (SEQ ID NO: 302) |

TABLE 9
GALV gp70 heavy chain antigen binding molecule variable domain amino acid
sequences.

| Clone | | SEQ ID |
|---|---|---|
| ID # | Amino Acid Sequence | NO. |
| 9G11 | QVQLQQPGAELVRPGSSVKLSCKASGYTFTNYWMDWVKQRPGQGLEWIGHIYPSDS | 303 |
| | ETHYIQKFKDKATLTVDKSSNTAYMQLSSLTSEDSAVYYCARDYYWGQGTTLTVSS | |
| 40A6 | EVQLVESGGGLVKPGGSLKLSCAASGFTFSSYVLSWVRQIPEKRLEWVATINSGGSYTY | 304 |
| | YPDSVKGRFTISRDNAKNTLYLQMSSLRSEDTAIYYCARHLIYYDYDGYYFDTWGQGTS | |
| | LTVSS | |
| 40A3 | DVQLQESGPDLVKPSQSLSLTCTVTDFSITSDYTWHWVRQFPGNKLEWMAYIHYSGG | 305 |
| | TNYNPSLRSRISITRDTSKNQFFLHMNSVTTEDTATYYCLYYGSRDAVDYWGQGTSVT | |
| | VSS | |
| 3C8 V2 | QVQLQQPGAELVRPGSSVKLSCKASGYTFTHYWMDWVKQRPGQGLEWIGHIYPSD | 306 |
| | GETHYNQKFRDKATLTVDKSSSTAYVOLSSLTSEDSAVYYCARDYYWGQGTTLTVSS | |
| 3C8 V1 | EVQLQQSGPELVKPGASVKISCKASGYTFTDYYMNWVKQSHGKSLEWIGDITPNKGD | 307 |
| | TNYNQKFKDRATLTVDKSSNTAYMELRSLTSEDSSVYYCARGKNYSGSSLHWYFDVW | |
| | GTGTTVTVSS | |
| 36H3 | EVQLVESGGDLVKPGGSLKLSCAASGFTFSNYGMSWVRQTPDKRLEWVATISYSGAY | 308 |
| | TYYPDSVKGRFTISRDNAKNTLYLQMGSLKSEDTAMFFCSRHVEYYDYDPYALDFWG | |
| | QATSVTVSS | |
| 35C11 | QVQLQQSGPELVRPGVSVKISCKVSGYTFTDYTMHWVKQSHAKSLEWIGLISTYSGKT | 309 |
| | NYNQKLKDKATMTVDKSSSTAYMELARLTSEDSAIFYCARGGLRDAMDYWGQGTSV | |
| | TVSS | |
| 2D3 | QVQLQQPGAELVRPGSSVKLSCKASGYTFTNYWMDWVKQRPGQGLEWIGNIYPSD | 310 |
| | SETHYNQKFRDKATLTVDKPSSTAYMQLSSLTSEDSAVYYCARDYYWGQGTTLTVSS | |
| 13E7 | QVQLQQPGAEFVKPGASVKLSCEASGYTFTSHWIHWVKQRPGRGLDWIGRIDPNSG | 311 |
| | GTKYNEKFKSKATLTVDKPSSTAYMQLSSLTSEDPAVHYCAREGYEGYLGYFDFWGQG | |
| | TTLTVSS | |
| 4F1 | QVQLQQPGAELVRPGSSVKLSCKASGYTFTSYWMDWMKQRPGQGLEWIGHIYPSD | 312 |
| | SETHYNQKFKDRATLTVDKSSSAAFMQLSSLTSEDSAVYYCARDYYWGQGTTLTVSS | |
| 9A1 | QVQLQQPGAELVRPGSSVRLSCKASGYTFTSYWMDWVKQRPGQGLEWIGNIYPSDS | 313 |
| | ETHYNQKFKDKATLTVDKSSSTAYMQLSSLTSEDSAVYYCARDYYWGQGTTLTVSS | |
| 8G8 | QVQLQQPGAELVRPGSSVKLSCKASGYTFTNYWMDWVKQRPGQGLEWIGNIYPSD | 314 |
| | SETHYNQNFKDKATLTVDKSSSTAYMQLSSLTSEDSAVYYCARDYYWGQGTTLTVSS | |

TABLE 10
GALV gp70 light chain antigen binding molecule variable domain amino acid
sequences.

| Clone | | SEQ ID |
|---|---|---|
| ID # | Amino Acid Sequence | NO. |
| 13E7 | DVQITQSPSYLAASPGETITINCRASKNISKYLAWYQEKPGKTNKLLIYSGSTLQSGNPSRF | 315 |
| | SASGSGTDFTLTISSLEPEDFAMYYCQQHYEYPYTFGGGTKLEIKLEIK | |
| 2D3 | QAVVTQESALTTSPGETVTLTCRSSTGAVTTSNYANWVQEKPDHLFTGLIGGTNNRALG | 316 |
| | VPARFSGSLIGDKAALTITGAQIEDEAIYFCALWYSNHLVFGGGTKLTVLQPK | |
| 35C11 | DIVLTQSPASLAVSLGQGATISCRASESVDSYGNSFMHWYQQKPGQPPKLLIYRASNLES | 317 |
| | GIPARFSGSGSGTDFTLTINPVEADDVATYYCLQSNEDPRTFGGGTKLEIK | |
| 36H3 | QIVLTQSPAIMSASPGEKVTMTCSTSSSVTYMHWYQQKSGTSPKRWIYDTSKLASGVPA | 318 |
| | RFSGSGSGTSYSLTISSMEAEDAATYYCQQWSSSPYTFGGGTKLEIK | |
| 3C8 | QAVVTQESALTTSPGETVTLTCRSNTGAVTTSNYANWVQERPDHLFTGLIGGTNNRVPG | 319 |
| | VPARFSGSLIGDKAALTITGAQTEDEAIYFCALWYSNHWVFGGGTKLTVLQPK | |
| 40A6 | DVVMTQTPLSLPVSLGDQASISCRSSQTLVHSNGNIYLHWYLQKPGQSPKLLIYKVSNRF | 320 |
| | SGVPDRFSGSGSGTDFTLKISRVETEDLGIYFCSQTTHVPPTFGGGTKLEIK | |
| 4F1 | QAVVTQESALTTSPGETVTLTCRSSTGAVTTSNYANWVQEKPDHLFTGLIGGTNNRAPG | 321 |
| | VPARFSGSLIGDKAALTITGAQTEDEAIYFCALWYSNHLVFGGGTKLTVLQPK | |
| 8G8 | QAVVTQESALTTSPGETVTLTCRSSTGAVTRSNYANWVQEKPDHLFTGLIGGTNNRALG | 322 |
| | VPARFSGSLIGDKAALTITGAQTEDEAIYFCALWYSNHLVFGGGTKLTVLQPK | |
| 9A1 | QAVVTQESALTTSPGETVTLTCRSSTGAVTRSNYANWVQEKPDHLFTGLIGGTNNRAPG | 323 |
| | VPARFSGSLIGDKAALTITGAQTEDEAIYFCALWYSNHLVFGGGTKLTVLQPK | |
| 9G11 | QAVVTQESALTTSPGETVTLTCRSSTGAVTTSNYANWVQEKPDHLFTGLIGGTNNRAPG | 324 |
| | VPARFSGSLIGDKAALTITGAQTEDEAIYFCALWYSSQLVFGGGTKLTVLQPK | |

TABLE 11
GALV gp70 binding antibody clones.

| Antibody | Heavy Chain Clone | Light Chain Clone |
|---|---|---|
| 1 | 13E7-HC | 13E7-LC |
| 2 | 2D3-HC1 | 2D3-LC1 |
| 3 | 35C11-HC | 35C11-LC |
| 4 | 36H3-HC1 | 36H3-LC |
| 5 | 3C8-HC1 | 3C8-LC |
| 6 | 3C8-HC2 | 3C8-LC |
| 7 | 40A3-HC | 36H3-LC |
| 8 | 40A6-HC | 40A6-LC |
| 9 | 4F1-HC | 4F1-LC1 |
| 10 | 8G8-HC2 | 8G8-LC2 |
| 11 | 9A1-HC3 | 9A1-LC1 |
| 12 | 9G11-HC | 9G11-LC |

In one embodiment, the antigen binding molecules of the present disclosure are antibodies and antigen binding fragments thereof. In certain embodiment, the antibodies of the present disclosure comprise at least one CDR set forth in Tables 7A-7C and 8A-8C. In certain embodiment, the antibodies of the present disclosure comprise at least one heavy chain variable region from Table 9. In certain embodiment, the antibodies of the present disclosure comprise at least one light chain variable region from Table 10. In certain embodiments, the antibodies of the present disclosure comprise the combination of heavy chain variable region and light chain variable region as shown in Table 11. In another aspect, the present disclosure provides hybridomas capable of producing the antibodies disclosed herein and methods of producing antibodies from hybridomas, as described herein and as known in the art.

Humanized antibodies are described herein and may be prepared by known techniques. In one embodiment, a humanized monoclonal antibody comprises the variable domain of a murine or rabbit antibody (or all or part of the antigen binding site thereof) and a constant domain derived from a human antibody. Alternatively, a humanized antibody fragment may comprise an antigen binding site of a murine or rabbit monoclonal antibody and a variable domain fragment (lacking the antigen binding site) derived from a human antibody. Procedures for the production of engineered monoclonal antibodies include those described in Riechmann et al., (1988) Nature 332:323, Liu et al., (1987) Proc. Nat. Acad. Sci. USA 84:3439, Larrick et al., (1989) Bio/Technology 7:934, and Winter et al., (1993) TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557; Padlan et al., (1995) FASEB J. 9:133-39; Tamura et al., (2000) J. Immunol. 164:1432-41; Zhang et al., (2005) Mol. Immunol. 42(12):1445-1451; Hwang et al., Methods. (2005) 36(1):35-42; Dall'Acqua et al., (2005) Methods 36(1):43-60; and Clark, (2000) Immunology Today 21(8):397-402.

An antigen binding molecule of the present invention can also be a fully human monoclonal antibody. Fully human monoclonal antibodies can be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., (1997) Curr. Opin. Biotechnol. 8:455-58).

Examples of techniques for production and use of transgenic animals for the production of human or partially human antibodies are described in U.S. Pat. Nos. 5,814,318, 5,569,825, and 5,545,806; Davis et al., Antibody Engineering: Methods and Protocols, (Lo, ed) Humana Press, NJ, 191-200 (2003); Kellermann et al., (2002) Curr Opin Biotechnol. 13:593-97; Russel et al., (2000) Infect Immun. 68:1820-26; Gallo et al., (2000) Eur J. Immun. 30:534-40; Davis et al., (1999) Cancer Metastasis Rev. 18:421-25; Green, (1999) J Immunol Methods 231:11-23; Jakobovits, (1998) Advanced Drug Delivery Reviews 31:33-42; Green et al., (1998) J Exp Med. 188:483-95; Jakobovits, (1998) Exp. Opin. Invest. Drugs. 7:607-14; Tsuda et al., (1997) Genomics, 42:413-21; Mendez et al., (1997) Nat. Genet. 15:146-56; Jakobovits, (1994) Curr Biol. 4:761-63; Arbones et al., (1994) Immunity 1:247-60; Green et al., (1994) Nat. Genet. 7:13-21; Jakobovits et al., (1993) Nature 362:255-58; Jakobovits et al., (1993) Proc Natl Acad Sci USA 90:2551-55; Chen et al., (1993) Intl Immunol 5:647-656; Choi et al., (1993) Nature Genetics 4:117-23; Fishwild et al., (1996) Nature Biotechnology 14:845-51; Lonberg et al., (1994) Nature 368: 856-59; Lonberg, (1994) Handbook of Experimental Pharmacology 113: 49-101; Neuberger, (1996) Nature Biotech 14:826; Taylor et al., (1992) Nucleic Acids Research 20:6287-95; Taylor et al., (1994) Intl Immunol 6:579-91; Tomizuka et al., (1997) Nature Genetics 16:133-43; Tomizuka et al., (2000) Proc Nat Acad Sci USA 97:722-27; Tuaillon et al., (1993) Proc Nat Acad Sci USA 90:3720-24; Tuaillon et al., (1994) J Immunol 152:2912-20.; Lonberg et al., (1994) Nature 368:856; Taylor et al., (1994) Intl Immunol 6:579; U.S. Pat. No. 5,877,397; Bruggemann et al., (1997) Curr. Opin. Biotechnol. 8:455-58; Jakobovits et al., (1995) Ann. N. Y. Acad. Sci. 764:525-35.

An additional method for obtaining antigen binding molecules of the invention is by the use of phage display, which is well-established for this purpose. See, e.g., Winter et al., (1994) Ann. Rev. Immunol. 12:433-55; Burton et al., (1994) Adv. Immunol 57:191-280. Human or murine immunoglobulin variable region gene combinatorial libraries can be created in phage vectors that can be screened to select Ig fragments (Fab, Fv, sFv, or multimers thereof) that bind the scFv-14, as well as molecules comprising this sequence and cells presenting such molecules. See, e.g., U.S. Pat. No. 5,223,409; Huse et al., (1989) Science 246:1275-81; Sastry et al., (1989) Proc. Natl. Acad. Sci. USA 86:5728-32; Alting-Mees et al., (1990) Strategies in Molecular Biology 3:1-9; Kang et al., (1991) Proc. Natl. Acad. Sci. USA 88:4363-66; Hoogenboom et al., (1992) J. Mol. Biol. 227:381-388; Schlebusch et al., (1997) Hybridoma 16:47-52 and references cited therein. For example, a library containing a plurality of polynucleotide sequences encoding Ig variable region fragments can be inserted into the genome of a filamentous bacteriophage, such as M13 or lambda phage (λImmunoZap™(H) and λImmunoZap™(L) vectors (Stratagene, La Jolla, Calif) can also be used in this approach) or a variant thereof, in frame with the sequence encoding a phage coat protein.

Briefly, mRNA is isolated from a B-cell population, and used to create heavy and light chain immunoglobulin cDNA expression libraries in the λImmunoZap™(H) and λImmunoZap™(L) and similar vectors. These vectors can be screened individually or co-expressed to form Fab fragments or antibodies. Positive plaques can subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers can be synthesized by one of ordinary skill in the art, or can be purchased from commercial sources, which also sell primers for mouse and human variable regions including, among others, primers for V_{H}, V_{L}, C_{H} and C_{L} regions). These primers can be used to amplify heavy or light chain variable regions, which can then be inserted into vectors. These vectors can then be introduced into E. coli, yeast, or mammalian-based systems for expression. Large amounts of a single-chain protein containing a fusion of the V_{H} and V_{L} domains can be produced using these methods.

Once cells producing the antigen binding molecules provided herein have been obtained using any of the above-described immunization and other techniques, the specific antibody genes can be cloned by isolating and amplifying DNA or mRNA therefrom according to standard procedures as described herein. The antibodies produced therefrom can be sequenced and the CDRs identified and the DNA coding for the CDRs can be manipulated as described previously to generate other antibodies according to the invention.

It will be understood by those of skill in the art that some proteins, such as antibodies, can undergo a variety of posttranslational modifications. The type and extent of these modifications often depends on the host cell line used to express the protein as well as the culture conditions. Such modifications can include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in, e.g., Harris, (1995) J Chromatog 705:129-34).

An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, e.g., Baines and Thorpe, (1992) in Methods in Molecular Biology, 10:79-104 (The Humana Press). Monoclonal antibodies can be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anti-constant region (light chain or heavy chain) antibody, and an anti-idiotype antibody.

Although the disclosed antigen binding molecules were produced in a mouse system, human, partially human, or humanized antibodies may be suitable for many applications, particularly those involving administration of the antibody to a human subject, other types of antigen binding molecules will be suitable for certain applications. Such antibodies can be prepared as described herein and form an aspect of the instant disclosure.

The instant disclosure provides antigen binding molecules that specifically bind to GALV gp70 protein and subsequences thereof, molecules comprising this sequence and cells presenting such molecules. Antigen binding molecules that cross compete with the antigen binding molecules disclosed herein form another aspect of the instant disclosure.

In some embodiments, the antibody or antigen binding molecule that specifically binds GALV gp70 protein binding domain binds the same or an overlapping epitope as a reference antibody disclosed herein. In certain embodiments, the antibody or antigen binding molecule binds the same or an overlapping epitope as a reference antibody.

### a) Antibodies

Antibodies (Ab) may include, without limitation, a glycoprotein immunoglobulin which binds specifically to an antigen. In general, an antibody may comprise at least two heavy chains (HC) and two light chains (LC) and may be interconnected by disulfide bonds, or an antigen binding molecule. Each HC chain comprises a heavy chain variable region (V_{H}) and a heavy chain constant region (CH). The heavy chain constant region may comprise three constant domains, CH1, CH2 and CH3. Each LC chain may comprise a light chain variable region (V_{L}) and a light chain constant region. The light chain constant region may comprise one constant domain, CL. The V_{H} and V_{L} regions may be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component of the classical complement system (C1q).

In some embodiments, the antigen binding molecules of the present invention specifically bind to at least a portion of scFv-14, molecules comprising the same or related sequences and cells presenting such molecules. In certain embodiments, an antigen binding molecule of the present disclosure specifically binds to at least a portion of scFv-14, as well as molecules comprising the same or similar sequences and cells presenting such molecules, with a K_{D} of less than 1×10⁻⁶ M, less than 1×10⁻⁷ M, less than 1×10⁻⁸ M, or less than 1×10⁻⁹ M. In one particular embodiment, an antigen binding molecule specifically binds to at least a portion of scFv-14, as well as molecules comprising these sequences and cells presenting such molecules, with a K_{D} of less than 1×10⁻⁷ M. In another embodiment, an antigen binding molecule specifically binds scFv-14, as well as molecules having the same or similar sequences, and cells presenting such molecules, with a K_{D} of less than 1×10⁻⁸ M. In some embodiments, an antigen binding molecule binds the scFv-14, as well as molecules comprising the same or similar sequences and cells presenting such molecules, with a K_{D} of about 1×10⁻⁷ M, about 2×10⁻⁷ M, about 3×10⁻⁷ M, about 4×10⁻⁷ M, about 5×10⁻⁷ M, about 6×10⁻⁷ M, about 7×10⁻⁷ M, about 8×10⁻⁷ M, about 9×10⁻⁷ M, about 1×10⁻⁸ M, about 2×10⁻⁸ M, about 3×10⁻⁸ M, about 4×10⁻⁸ M, about 5×10⁻⁸ M, about 6×10⁻⁸ M, about 7×10⁻⁸ M, about 8×10⁻⁸ M, about 9×10⁻⁸ M, about 1×10⁻⁹ M, about 2×10⁻⁹ M, about 3×10⁻⁹ M, about 4×10⁻⁹ M, about 5×10⁻⁹ M, about 6×10⁻⁹ M, about 7×10⁻⁹ M, about 8×10⁻⁹ M, about 9×10⁻⁹ M, about 1×10⁻¹⁰ M, or about 5×10⁻¹⁰ M. K_{D} can be calculated using standard methodologies, as described herein and elsewhere in the art.

In specific embodiments, an antigen binding molecule of the instant disclosure is identified in Tables 3-4 and each comprises the identified heavy and light chain amino acid sequences described in those Tables.

### b) ScFv

In various embodiments, a scFv may be a fusion protein comprising a V_{H} and a V_{L} of an immunoglobulin or an analog. In various embodiments, the V_{H} and the V_{L} may be connected by a linker. In various embodiments, an scFv does not include constant regions that are normally present in antibodies.

In various embodiments scFvs facilitate phage display, where it is convenient to express the antigen-binding domain as a single peptide. In other embodiments, a scFv may be created directly from subcloned heavy and light chains derived from a hybridoma. ScFvs may be used for a variety of different purposes. In various embodiments, scFvs may be incorporated into flow cytometry and immunohistochemistry diagnostic assays. In other embodiments, scFvs may be used as antigen-binding domains of artificial T cell receptors (chimeric antigen receptor).

### c) Linkers

Linker sequences may be peptide-based when employed in biotechnological and biotherapeutic applications and may serve a range of scientifically-relevant applications. For example, a linker can be used as simply a spacer moiety in order to impart a desired structural and/or functional property to a larger molecule. In another example, a linker can impart little or no structural or functional properties to a larger molecule, but can be used simply as a distinguishing feature (e.g., a “marker” or “biomarker” or “tag”), uniquely identifying a larger molecule. In still another example, a linker can be used to impart a recognizable feature that can serve as a binding site for an antibody directed against a larger molecule comprising the linker sequence.

In various embodiments, a linker may comprise a sequence of amino acids forming a peptide. For example, a linker may comprise a peptide sequence between about 20 to about 30 amino acids. In some embodiments, the linker may comprise a peptide sequence of about 25 amino acids. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids. In some embodiments, the linker comprises between about 8 amino acids and about 18 amino acids (e.g., 10 amino acids).

In various embodiments, a linker may comprise a flexible portion. For example, the flexible portion may be rich in glycine residues. In various embodiments, a linker may be soluble or partially soluble. For example, a linker may comprise one or more serine and/or threonine residues. In various embodiments, a linker may connect an N-terminus of a V_{H} with a C-terminus of a V_{L}. In other embodiments, a linker may connect a C-terminus of a V_{H} with an N-terminus of a VL.

When a linker sequence is used as a distinguishing, detectable or identifiable feature of a larger molecule, an antibody that specifically binds the linker sequence, to the exclusion of other sequences present in the larger molecule, the antibody can serve as a detection agent. Such antibodies can be labeled with a moiety that is detectable under certain conditions. Additional applications for such an antibody include purification and isolation of a molecule comprising the linker, characterization of a molecule in a particular setting, enrichment of the concentration of a population of molecules comprising and/or presenting the linker, and therapeutic applications as well.

In 1993, Whitlow et al. disclosed a synthetic linker peptide comprising the amino acid sequence GSTSGSGKPGSGEGSTKG (SEQ ID NO: 121) (Whitlow et al., (1993) Prot. Eng. 6(8):989-95). The disclosed peptide was studied as a component of an scFv, and was designed to remove a proteolytic site identified in a previous linker peptide. Whitlow et al. concluded that this newly-designed synthetic linker peptide was more stable to proteolysis in vitro when compared to the prior linker peptide upon which its sequence was based, and also showed less aggregation compared to the same prior linker. Whitlow et al. did not disclose any antigen binding molecules directed to their second-generation linker peptide.

In various embodiments, a “Whitlow” linker sequence may be included in an scFv antigen binding molecule described herein. In various embodiments, the linker sequence may be included in a larger amino acid sequence that also includes a heavy chain amino acid sequence and a light chain amino acid sequence.

There are various other linkers available in the art that may be suitable. One example is a “G4S linker” with the sequence: (Gly-Gly-Gly-Gly-Ser)3 (SEQ ID NO: 126). In various embodiments, a linker may include SEQ ID NO: 126 and 1 or more additional Gly residues. In various embodiments, a linker may include SEQ ID NO: 126 and 1 or fewer Gly residues.

Linkers can vary in length based on the number of Gly residues.

### V. Polynucleotides Encoding Antibodies and Antigen Binding Molecules

Anti-CD20 scFv-14 Binding Molecules

In various embodiments, a polynucleotide may encode antigen binding molecules (SEQ ID NOs: 1-120). In various embodiments, a polynucleotide may encode antigen binding molecules (SEQ ID NOs: 1-10). In various embodiments, a polynucleotide may encode antigen binding molecules (SEQ ID NOs: 11-20).

In some embodiments, a polynucleotide of the present invention encodes an antigen binding molecule, wherein the antigen binding molecule comprises a heavy chain variable region amino acid sequence that is at least about 75%, at least about 85%, at least about 85%, at least about 90%, at least about 95%, at least about 96%, at least about 97%, at least about 98%, at least about 99%, or 100% identical to a heavy chain variable region amino acid sequence selected from the group consisting of SEQ ID NOs: 1-10.

In some embodiments, a polynucleotide of the present invention encodes antigen binding molecule, wherein the antigen binding molecule comprises a light chain variable amino acid sequence that is at least about 75%, at least about 85%, at least about 85%, at least about 90%, at least about 95%, at least about 96%, at least about 97%, at least about 98%, at least about 99%, or 100% identical to a light chain variable region amino acid sequence selected from the group consisting of SEQ ID NOs: 11-20.

As will be appreciated by those of skill in the art, variations of the disclosed polynucleotide sequences are possible due to the degeneracy of the genetic code. Such variants of the disclosed polynucleotide sequences thus form an aspect of the instant disclosure.

### GALV Gp70 Binding Molecules

In various embodiments, a polynucleotide may encode antigen binding molecules (SEQ ID NOs: 127-324). In various embodiments, a polynucleotide may encode antigen binding molecules (SEQ ID NOs: 303-314). In various embodiments, a polynucleotide may encode antigen binding molecules (SEQ ID NOs: 315-324).

In some embodiments, a polynucleotide of the present invention encodes an antigen binding molecule, wherein the antigen binding molecule comprises a heavy chain variable region amino acid sequence that is at least about 75%, at least about 85%, at least about 85%, at least about 90%, at least about 95%, at least about 96%, at least about 97%, at least about 98%, at least about 99%, or 100% identical to a heavy chain variable region amino acid sequence selected from the group consisting of SEQ ID NOs: 303-314.

In some embodiments, a polynucleotide of the present invention encodes antigen binding molecule, wherein the antigen binding molecule comprises a light chain variable amino acid sequence that is at least about 75%, at least about 85%, at least about 85%, at least about 90%, at least about 95%, at least about 96%, at least about 97%, at least about 98%, at least about 99%, or 100% identical to a light chain variable region amino acid sequence selected from the group consisting of SEQ ID NOs: 315-324.

As will be appreciated by those of skill in the art, variations of the disclosed polynucleotide sequences are possible due to the degeneracy of the genetic code. Such variants of the disclosed polynucleotide sequences thus form an aspect of the instant disclosure.

TABLE 12
GALV gp70 heavy chain antigen binding molecule variable domain nucleic
acid sequences.

| Clone | | SEQ ID |
|---|---|---|
| ID # | Representative Nucleic Acid Sequence | NO. |
| 9G11 | CAGGTCCAACTGCAGCAGCCTGGGGCTGAGCTGGTGAGGCCTGGG | 347 |
| | TCTTCAGTGAAGCTGTCCTGCAAGGCTTCTGGCTACACCTTCACCA | |
| | ACTACTGGATGGATTGGGTGAAGCAGAGGCCTGGACAAGGCCTTG | |
| | AATGGATTGGTCACATTTACCCTTCTGATAGTGAAACTCACTACAT | |
| | TCAAAAGTTCAAGGACAAGGCCACATTGACTGTAGACAAATCCTC | |
| | CAACACAGCCTACATGCAGCTCAGCAGCCTGACATCTGAGGACTC | |
| | TGCGGTCTATTACTGTGCAAGAGATTACTACTGGGGCCAAGGCAC | |
| | CACTCTCACAGTCTCCTCA | |
| 40A6 | GAAGTGCAGCTGGTGGAGTCTGGGGGAGGCTTAGTGAAGCCTGGA | 326 |
| | GGGTCCCTGAAACTCTCCTGTGCAGCCTCTGGATTCACTTTCAGTA | |
| | GTTATGTCCTGTCTTGGGTTCGCCAGATTCCGGAGAAGAGGCTGGA | |
| | GTGGGTCGCAACCATTAATAGTGGTGGTAGTTACACTTACTATCCA | |
| | GACAGTGTGAAGGGTCGATTCACCATCTCCAGAGACAATGCCAAG | |
| | AACACCCTGTACCTGCAAATGAGCAGTCTGAGGTCTGAGGACACG | |
| | GCCATATATTACTGTGCACGACATCTCATCTACTATGATTACGACG | |
| | GTTACTACTTTGACACCTGGGGCCAAGGCACCTCTCTCACAGTCTC | |
| | CTCA | |
| 40A3 | GATGTGCAGCTTCAGGAGTCAGGACCTGACCTAGTGAAACCTTCTC | 327 |
| | AGTCGCTTTCACTCACCTGCACTGTCACTGACTTCTCCATCACCAG | |
| | TGATTATACCTGGCACTGGGTCCGGCAGTTTCCAGGAAACAAACT | |
| | GGAATGGATGGCCTACATACACTACAGTGGTGGCACTAACTACAA | |
| | TCCATCTCTCAGAAGTCGAATCTCTATCACTCGAGACACATCCAAG | |
| | AACCAGTTCTTCCTGCATATGAATTCTGTGACTACTGAGGACACAG | |
| | CCACATATTACTGTCTTTACTACGGTAGTAGAGATGCTGTGGACTA | |
| | CTGGGGTCAAGGAACCTCAGTCACCGTCTCCTCA | |
| 3C8 V2 | CAGGTCCAACTACAGCAACCTGGGGCTGAACTGGTGAGGCCTGGG | 328 |
| | TCTTCAGTGAAACTGTCCTGCAAGGCTTCTGGCTACACCTTCACCC | |
| | ACTACTGGATGGATTGGGTGAAGCAGAGGCCTGGACAAGGCCTTG | |
| | AATGGATTGGTCACATTTACCCTTCTGATGGTGAAACTCACTACAA | |
| | TCAAAAGTTCAGGGACAAGGCCACATTGACTGTAGACAAATCCTC | |
| | CAGCACAGCCTACGTGCAGCTCAGCAGCCTGACATCTGAGGACTC | |
| | TGCGGTCTATTATTGTGCAAGAGACTATTACTGGGGCCAAGGCACC | |
| | ACTCTCACAGTCTCCTCA | |
| 3C8 V1 | GAAGTCCAGCTGCARCAGTCTGGACCTGAGCTGGTGAAGCCTGGG | 329 |
| | GCTTCTGTGAAGATATCCTGTAAGGCTTCTGGATACACGTTCACTG | |
| | ACTACTACATGAACTGGGTGAAGCAGAGCCATGGAAAGAGCCTTG | |
| | AGTGGATTGGAGATATTACTCCTAACAAGGGTGATACTAACTACA | |
| | ACCAGAAGTTCAAGGACAGGGCCACATTGACTGTCGACAAGTCCT | |
| | CCAACACAGCCTACATGGAGCTCCGCAGCCTGACATCTGAGGACT | |
| | CTTCAGTCTATTACTGTGCAAGAGGGAAAAATTACTCCGGTAGTAG | |
| | CCTTCACTGGTACTTCGATGTCTGGGGCACAGGGACCACGGTCACC | |
| | GTCTCCTCA | |
| 36H3 | GAGGTGCAGCTGGTGGAGTCTGGGGGAGACTTAGTGAAGCCTGGA | 330 |
| | GGGTCCCTGAAACTCTCCTGTGCAGCCTCTGGATTCACTTTCAGTA | |
| | ACTATGGCATGTCTTGGGTTCGCCAGACTCCAGACAAGAGACTGG | |
| | AGTGGGTCGCAACCATTAGTTATAGTGGTGCTTACACCTACTATCC | |
| | AGACAGTGTGAAGGGGCGATTCACCATCTCCAGAGACAATGCCAA | |
| | GAACACCCTGTACCTGCAAATGGGCAGTCTGAAGTCTGAGGACAC | |
| | AGCCATGTTTTTCTGTTCAAGACATGTTGAGTACTATGATTACGAC | |
| | CCTTATGCTTTGGACTTCTGGGGTCAAGCAACCTCAGTCACCGTCT | |
| | CCTCA | |
| 35C11 | CAGGTCCAGCTGCAGCAGTCTGGGCCTGAGCTGGTGAGGCCTGGG | 331 |
| | GTCTCAGTGAAGATTTCCTGCAAGGTTTCCGGCTACACATTCACTG | |
| | ATTATACTATGCATTGGGTGAAGCAGAGTCATGCAAAGAGTCTAG | |
| | AGTGGATTGGACTTATTAGCACTTACTCTGGTAAAACAAACTACAA | |
| | CCAGAAACTTAAGGACAAGGCCACAATGACTGTGGACAAATCCTC | |
| | CAGCACAGCCTATATGGAACTTGCCAGATTGACATCTGAGGATTCT | |
| | GCCATCTTTTACTGTGCAAGAGGGGGATTAAGGGATGCTATGGAC | |
| | TACTGGGGTCAAGGAACCTCAGTCACCGTCTCCTCA | |
| 2D3 | CAGGTCCAACTGCAGCAGCCTGGGGCTGAGCTGGTGAGGCCTGGG | 332 |
| | TCTTCAGTGAAACTGTCCTGCAAGGCTTCTGGCTACACCTTCACCA | |
| | ACTACTGGATGGATTGGGTGAAACAGAGGCCTGGACAAGGCCTTG | |
| | AATGGATTGGTAACATTTACCCTTCTGATAGTGAAACTCACTACAA | |
| | TCAAAAGTTCAGGGACAAGGCCACATTGACTGTAGACAAACCCTC | |
| | CAGCACAGCCTACATGCAGCTCAGCAGCCTGACATCTGAGGACTC | |
| | TGCGGTCTATTATTGTGCAAGAGACTATTACTGGGGCCAAGGCACC | |
| | ACTCTCACAGTCTCCTCA | |
| 13E7 | CAGGTCCAACTGCAGCAGCCTGGGGCTGAGTTTGTGAAGCCTGGG | 333 |
| | GCTTCAGTGAAGCTGTCCTGTGAGGCTTCTGGCTACACCTTCACCA | |
| | GCCACTGGATACACTGGGTGAAGCAGAGGCCTGGACGAGGCCTTG | |
| | ACTGGATTGGAAGGATTGATCCTAATAGTGGTGGTACTAAGTACA | |
| | ATGAGAAGTTCAAGAGCAAGGCCACACTGACTGTAGACAAACCCT | |
| | CCAGCACAGCCTACATGCAGCTCAGCAGCCTGACATCTGAGGACC | |
| | CTGCGGTCCATTACTGTGCAAGAGAGGGCTATGAGGGTTACTTGG | |
| | GTTACTTTGACTTCTGGGGCCAAGGCACCACTCTCACAGTTTCCTC | |
| | A | |
| 4F | CAGGTCCAACTGCAGCAGCCTGGGGCTGAGCTGGTGAGGCCTGGG | 334 |
| | TCTTCAGTGAAACTATCCTGCAAGGCTTCTGGCTACACCTTCACCA | |
| | GCTACTGGATGGATTGGATGAAACAGAGGCCTGGACAAGGCCTTG | |
| | AATGGATTGGTCACATTTACCCTTCTGATAGTGAAACTCACTACAA | |
| | TCAAAAGTTCAAGGACAGGGCCACATTGACTGTAGACAAATCCTC | |
| | CAGCGCAGCCTTCATGCAACTCAGCAGCCTGACATCTGAGGACTCT | |
| | GCGGTCTATTACTGTGCAAGAGATTACTACTGGGGCCAAGGCACC | |
| | ACTCTCACAGTCTCCTCA | |
| 9A1 | CAGGTCCAACTACAGCAACCTGGGGCTGAGCTGGTGAGGCCTGGG | 335 |
| | TCTTCAGTGAGACTGTCCTGCAAGGCTTCTGGCTACACCTTCACCA | |
| | GCTACTGGATGGATTGGGTGAAGCAGAGGCCTGGACAGGGCCTTG | |
| | AATGGATTGGTAACATTTACCCTTCTGATAGTGAAACTCACTACAA | |
| | TCAAAAGTTCAAGGACAAGGCCACATTGACTGTAGACAAATCCTC | |
| | CAGCACAGCCTACATGCAGCTCAGCAGCCTGACATCTGAGGACTC | |
| | TGCGGTCTATTACTGTGCAAGAGACTACTACTGGGGCCAAGGCAC | |
| | CACTCTCACAGTCTCCTCA | |
| 8G8 | CAGGTCCAACTACAGCAACCTGGGGCTGAGCTGGTGAGGCCTGGG | 336 |
| | TCTTCAGTGAAACTGTCCTGCAAGGCTTCTGGCTACACCTTCACCA | |
| | ACTACTGGATGGATTGGGTGAAGCAGAGGCCTGGACAGGGCCTTG | |
| | AATGGATTGGTAACATTTACCCTTCTGATAGTGAAACTCACTACAA | |
| | TCAAAACTTCAAGGACAAGGCCACATTGACTGTAGACAAATCCTC | |
| | CAGCACAGCCTACATGCAGCTCAGCAGCCTGACATCTGAGGACTC | |
| | TGCGGTCTATTACTGTGCAAGAGACTACTACTGGGGCCAAGGCAC | |
| | CACTCTCACAGTCTCCTCA | |

TABLE 13
GALV gp70 light chain antigen binding molecule variable domain nucleic acid
sequences.

| Clone | | SEQ ID |
|---|---|---|
| ID # | Representative Nucleic Acid Sequence | NO. |
| 13E7 | GATGTCCAGATACTCCAGTCTCCATCTTATCTTGCTGCATCTCCTGGA | 337 |
| | GAAACCATTACTATTAATTGCAGGGCAAGTAAGAACATTAGCAAAT | |
| | ATTTAGCCTGGTATCAAGAGAAACCTGGGAAAACTAATAAACTTCTT | |
| | ATCTACTCTGGATCCACTTTGCAATCTGGAAATCCATCAAGGTTCAG | |
| | TGCCAGTGGATCTGGTACAGATTTCACTCTCACCATCAGTAGCCTGG | |
| | AGCCTGAAGATTTTGCAATGTATTACTGTCAACAGCATTATGAATAC | |
| | CCGTACACGTTCGGAGGGGGGACCAAGCTGGAAATAAAA | |
| 2D3 | CAGGCTGTTGTGACTCAGGAATCTGCACTCACCACATCACCTGGTGA | 338 |
| | AACAGTCACACTCACTTGTCGCTCAAGTACTGGGGCTGTTACAACTA | |
| | GTAACTATGCCAACTGGGTCCAAGAAAAACCAGATCATTTATTCACT | |
| | GGTCTAATAGGTGGTACCAACAACCGAGCTCTAGGTGTTCCTGCCAG | |
| | ATTCTCAGGCTCCCTGATTGGAGACAAGGCTGCCCTCACCATCACAG | |
| | GGGCACAGATTGAGGATGAGGCAATATATTTCTGTGCTCTATGGTAC | |
| | AGCAACCATTTGGTGTTCGGTGGAGGAACCAAACTGACTGTCCTA | |
| 35C11 | GACATTGTGCTGACCCAATCTCCAGCTTCTTTGGCTGTGTCTCTAGG | 339 |
| | GCAGGGGGCCACCATATCCTGCAGAGCCAGTGAAAGTGTTGATAGT | |
| | TATGGCAATAGTTTTATGCACTGGTACCAGCAGAAACCAGGACAGC | |
| | CACCCAAACTCCTCATCTATCGTGCATCCAACCTAGAATCTGGGATC | |
| | CCTGCCAGGTTCAGTGGCAGTGGGTCTGGGACAGACTTCACCCTCAC | |
| | CATTAATCCTGTGGAGGCTGATGATGTTGCAACCTATTACTGTCTGC | |
| | AAAGTAATGAGGATCCTCGGACGTTCGGTGGAGGCACCAAGCTGGA | |
| | AATCAAA | |
| 36H3 | CAAATTGTTCTCACCCAGTCTCCAGCAATCATGTCTGCATCTCCAGG | 340 |
| | GGAGAAGGTCACCATGACCTGCAGTACCAGCTCAAGTGTAACTTAT | |
| | ATGCACTGGTACCAGCAGAAGTCAGGCACCTCCCCCAAAAGATGGA | |
| | TTTATGACACATCCAAACTGGCTTCTGGAGTCCCTGCTCGCTTCAGT | |
| | GGCAGTGGGTCTGGGACCTCTTACTCTCTCACAATCAGCAGCATGGA | |
| | GGCTGAAGATGCTGCCACTTATTACTGCCAGCAGTGGAGTAGTAGCC | |
| | CATACACGTTCGGAGGGGGGACCAAGCTGGAAATAAAA | |
| 3C8 | CAGGCTGTTGTAACTCAGGAATCTGCACTCACCACATCACCTGGTGA | 341 |
| | AACAGTCACACTCACTTGTCGCTCAAATACTGGGGCTGTTACAACCA | |
| | GTAACTATGCCAACTGGGTCCAAGAAAGACCAGATCATTTATTCACT | |
| | GGTCTAATAGGTGGTACCAACAACCGAGTTCCAGGTGTTCCTGCCAG | |
| | ATTCTCAGGCTCCCTGATTGGAGACAAGGCTGCCCTCACCATCACAG | |
| | GGGCACAGACTGAGGATGAGGCAATATATTTCTGTGCTCTATGGTAC | |
| | AGCAACCATTGGGTGTTCGGTGGAGGAACCAAACTGACTGTCCTA | |
| 40A6 | GATGTTGTGATGACCCAAACTCCACTCTCCCTGCCTGTCAGTCTTGG | 342 |
| | AGATCAAGCCTCCATCTCTTGCAGATCTAGTCAGACCCTTGTTCACA | |
| | GTAATGGAAATATCTATTTACATTGGTACCTGCAGAAGCCAGGCCAG | |
| | TCTCCAAAGCTCCTGATCTACAAAGTTTCCAACCGATTTTCTGGAGT | |
| | CCCAGACAGGTTCAGTGGCAGTGGATCAGGGACAGATTTCACACTC | |
| | AAGATCAGCAGAGTGGAGACTGAGGATCTGGGAATTTATTTCTGCTC | |
| | TCAAACCACACATGTTCCTCCGACGTTCGGTGGAGGCACCAAACTGG | |
| | AAATCAAA | |
| 4F1 | CAGGCTGTTGTGACTCAGGAATCTGCACTCACCACATCACCTGGTGA | 343 |
| | AACAGTCACACTCACTTGTCGCTCAAGTACTGGGGCTGTTACAACTA | |
| | GTAACTATGCCAACTGGGTCCAAGAAAAACCAGATCATTTATTCACT | |
| | GGTCTAATAGGTGGTACCAACAACCGAGCTCCAGGTGTTCCTGCCAG | |
| | ATTCTCAGGCTCCCTGATTGGAGACAAGGCTGCCCTCACCATCACAG | |
| | GGGCACAGACTGAGGATGAGGCAATATATTTCTGTGCTCTATGGTAC | |
| | AGCAACCATTTGGTGTTCGGTGGAGGAACCAAACTGACTGTCCTA | |
| 8G8 | CAGGCTGTTGTGACTCAGGAATCTGCACTCACCACATCACCTGGTGA | 344 |
| | AACAGTCACACTCACTTGTCGCTCAAGTACTGGGGCTGTTACAAGGA | |
| | GTAACTATGCCAACTGGGTCCAAGAAAAACCAGATCATTTATTCACT | |
| | GGTCTAATAGGTGGTACCAACAATCGAGCTCTAGGTGTTCCTGCCAG | |
| | ATTCTCAGGCTCCCTGATTGGAGACAAGGCTGCCCTCACCATCACAG | |
| | GGGCACAGACTGAGGATGAGGCAATATATTTCTGTGCTCTATGGTAC | |
| | AGCAACCATTTGGTGTTCGGTGGGGGAACCAAACTGACTGTCCTA | |
| 9A1 | CAGGCTGTTGTGACTCAGGAATCTGCACTCACCACATCACCTGGTGA | 345 |
| | AACAGTCACACTCACTTGTCGCTCAAGTACTGGGGCTGTTACAAGGA | |
| | GTAACTATGCCAACTGGGTCCAAGAAAAACCAGATCATTTATTCACT | |
| | GGTCTAATAGGTGGTACCAACAACCGAGCTCCAGGTGTTCCTGCCAG | |
| | ATTCTCAGGCTCCCTGATTGGAGACAAGGCTGCCCTCACCATCACAG | |
| | GGGCACAGACTGAGGATGAGGCAATATATTTCTGTGCTCTATGGTAC | |
| | AGCAACCATTTGGTGTTCGGTGGAGGAACCAAACTGACTGTCCTA | |
| 9G11 | CAGGCTGTTGTGACTCAGGAATCTGCACTCACCACATCACCTGGTGA | 346 |
| | AACAGTCACACTCACTTGTCGCTCAAGTACTGGGGCTGTTACAACTA | |
| | GTAACTATGCCAACTGGGTCCAAGAAAAACCAGATCATTTATTCACT | |
| | GGTCTAATAGGTGGTACCAACAACCGAGCTCCAGGTGTTCCTGCCAG | |
| | ATTCTCAGGCTCCCTGATTGGAGACAAGGCTGCCCTCACCATCACAG | |
| | GGGCACAGACTGAGGATGAGGCAATATATTTCTGTGCTCTATGGTAC | |
| | AGTAGCCAATTGGTGTTCGGTGGAGGAACCAAACTGACTGTCCTA | |

### VI. Vectors, Cells, and Pharmaceutical Compositions

In certain aspects, provided herein are vectors comprising a polynucleotide of the present disclosure. In some embodiments, the present invention is directed to a vector or a set of vectors comprising a polynucleotide encoding an antibody or antigen binding molecule that specifically binds anti-CD20 scFv-14 (SEQ ID NOs: 1-120). In some embodiments, the present invention is directed to a vector or a set of vectors comprising a polynucleotide encoding an antibody or antigen binding molecule that specifically binds GALV gp70 (SEQ ID NOs.127-324).

Any vector known in the art can be suitable for expressing the antibodies and antigen binding molecules of the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

In other aspects, provided herein are cells comprising a polynucleotide or a vector of the present invention. In some embodiments, the present invention is directed to cells, in vitro cells, comprising a polynucleotide encoding an antigen binding molecule, as described herein. In some embodiments, the present invention is directed to cells, e.g., in vitro cells, comprising a polynucleotide encoding an antibody or an antigen binding molecule thereof that specifically binds to anti-CD20 scFv-14 or GALV gp70 protein, molecules comprising these sequences and cells presenting such molecules, as disclosed herein.

Any cell can be used as a host cell for the polynucleotides and vectors encoding all or a fragment of the antibodies and antigen binding molecules of the present invention. In some embodiments, a host cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobacteriaceae such as Escherichia, e.g., E. coli; Bacilli such as B. subtilis and B. licheniformis; Pseudomonas such as P. aeruginosa; and Streptomyces. In some embodiments, a host cell is a mammalian cell, such as a human cell. In some embodiments, a host cell is a CHO cell and in other embodiments, a host cell is a sP2/0 or other murine cell. A host cell of the present invention can be obtained through any source known in the art.

Other aspects of the present disclosure may be directed to compositions comprising a polynucleotide described herein, a vector described herein, an antibody an antigen binding molecule described herein, and/or an in vitro cell described herein. In some embodiments, the composition comprises a pharmaceutically acceptable carrier, diluent, solubilizer, emulsifier, preservative and/or adjuvant. In some embodiments, the composition comprises an excipient.

In one embodiment, the composition comprises a polynucleotide encoding an antibody or antigen binding molecule that specifically binds to that specifically binds to anti-CD20 scFV-14 or GALV gp70 protein, and molecules comprising these sequences and cells presenting such molecules. In another embodiment, the composition comprises an in vitro cell comprising a polynucleotide encoding an antibody or an antigen binding molecule thereof encoded by a polynucleotide disclosed herein.

In some embodiments, the composition comprises more than one different antibody or antigen binding molecule that specifically binds to anti-CD20 scFv-14 or GALV gp70 protein, and molecules comprising these sequences and cells presenting such molecules. In some embodiments, the composition includes more than one antibody or antigen binding molecule that specifically binds to anti-CD20 scFv-14 or GALV gp70 protein, and molecules comprising these sequences and cells presenting such molecules, wherein the antibodies or antigen binding molecules bind more than one epitope. In some embodiments, the antibodies or antigen binding molecules will not compete with one another for binding to that epitope. In some embodiments, two or more of the antibodies or antigen binding molecules provided herein are combined together in a pharmaceutical composition. Preferably such a composition will be suitable for administration to a subject, including a human.

### VII. Exemplary Methods

The following section describes various exemplary methods of using the disclosed antigen binding molecules herein. Any of the antigen binding molecules, and fragments thereof, disclosed herein including those described in the Tables, Figures, and the attached Sequence Listing may be employed in the disclosed methods.

In some of the disclosed methods T cells can be employed. Such T cells can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

In various embodiments, the antigen binding molecule specifically binds to at least a portion of scFv-14 and/or molecules comprising similar sequences and cells presenting such sequences. In various further embodiments, the antigen binding molecule specifically binds to at least a portion of a GALV gp70 protein and/or molecules comprising similar sequences and viral particles presenting such sequences. In various embodiments, the antigen binding molecule may comprise one or more of (a) a light chain CDR1, (b) a light chain CDR2, (c) a light chain CDR3, (d) a heavy chain CDR1, (e) a heavy chain CDR2, and (f) a heavy chain CDR3. In various embodiments, a light chain and the heavy chain may be connected by a linker.

In various embodiments, an antigen binding molecule may comprise a variable heavy chain. In various embodiments of anti-CD20 scFv-14 binding molecules, the variable heavy chain variable region may comprise one of SEQ ID NOs: 1-10. In various embodiments, an antigen binding molecule can be employed which comprises a V_{H} amino acid sequence that is at least about 70%, at least about 75%, at least about 80%, at least about 85%, at least about 90%, at least about 95%, at least about 96%, at least about 97%, at least about 98%, at least about 99%, or about 100% identical to a V_{H} of an antigen binding molecule of claim disclosed herein (e.g., an antigen binding molecule comprising a V_{H} sequence comprising SEQ ID NO: 1-10.

In various embodiments, an antigen binding molecule may comprise a variable heavy chain. In various embodiments of GALV gp70 binding molecules, the variable heavy chain variable region may comprise one of SEQ ID NOs: 303-314. In various embodiments, an antigen binding molecule can be employed which comprises a V_{H} amino acid sequence that is at least about 70%, at least about 75%, at least about 80%, at least about 85%, at least about 90%, at least about 95%, at least about 96%, at least about 97%, at least about 98%, at least about 99%, or about 100% identical to a V_{H} of an antigen binding molecule of claim disclosed herein (e.g., an antigen binding molecule comprising a V_{H} sequence comprising SEQ ID NO: 303-314).

In various embodiments of anti-CD20 scFv-14 binding molecules a variable heavy chain may comprise one of a CDR1, wherein the CDR1 comprises one of SEQ ID NOs: 21-41. In various embodiments a heavy chain may comprise one of a CDR2, wherein the CDR2 comprises one of SEQ ID NOs: 42-65. In various embodiments a heavy chain may comprise one of a CDR3, wherein the CDR3 comprises one of SEQ ID NOs: 66-85.

In various embodiments of GALV gp70 binding molecules a variable heavy chain may comprise one of a CDR1, wherein the CDR1 comprises one of SEQ ID NOs: 127-138, 157-168, and 187-198. In various embodiments a heavy chain may comprise one of a CDR2, wherein the CDR2 comprises one of SEQ ID NOs: 139-150, 169-180, and 199-210. In various embodiments a heavy chain may comprise one of a CDR3, wherein the CDR3 comprises one of SEQ ID NOs: 151-156, 181-186, 211-222 and DYY.

In various embodiments of anti-CD20 scFv-14 binding molecules, a light chain may comprise one of SEQ ID NOs: 11-20. In various embodiments, an antigen binding molecule can be employed which comprises a V_{L} amino acid sequence that is at least about 70%, at least about 75%, at least about 80%, at least about 85%, at least about 90%, at least about 95%, at least about 96%, at least about 97%, at least about 98%, at least about 99%, or about 100% identical to a V_{L} of an antigen binding molecule of claim disclosed herein (e.g., an antigen binding molecule comprising a V_{L} sequence comprising SEQ ID NO: 11-20).

In various embodiments of GALV gp70 binding molecules, a light chain may comprise one of SEQ ID NOs: 315-324. In various embodiments, an antigen binding molecule can be employed which comprises a V_{L} amino acid sequence that is at least about 70%, at least about 75%, at least about 80%, at least about 85%, at least about 90%, at least about 95%, at least about 96%, at least about 97%, at least about 98%, at least about 99%, or about 100% identical to a V_{L} of an antigen binding molecule of claim disclosed herein (e.g., an antigen binding molecule comprising a V_{L} sequence comprising SEQ ID NO: 315-324).

In various embodiments of anti-CD20 scFv-14 binding molecules, a variable light chain may comprise one of a CDR1, wherein the CDR1 comprises one of SEQ ID NOs: 86-99. In various embodiments a light chain may comprise one of a CDR2, wherein the CDR2 comprises one of SEQ ID NOs: 100-111. In various embodiments a light chain may comprise one of a CDR3, wherein the CDR3 comprises one of SEQ ID NOs: 112-120.

In various embodiments of GALV gp70 binding molecules, a variable light chain may comprise one of a CDR1, wherein the CDR1 comprises one of SEQ ID NOs: 223-232, 253-262, and 283-292. In various embodiments a light chain may comprise one of a CDR2, wherein the CDR2 comprises one of SEQ ID NOs: 233-242, 263-272, SGS, GTN, RAS, DTS, and KVS. In various embodiments a light chain may comprise one of a CDR3, wherein the CDR3 comprises one of SEQ ID NOs: 243-252, 273-282, and 293-302.

In various embodiments, a variable light chain may be linked to a variable heavy chain by a linker (e.g., a Whitlow linker).

In further embodiments of the disclosed methods, the antigen binding molecule comprises one or more of (a) a light chain CDR1, (b) a light chain CDR2, (c) a light chain CDR3, (d) a heavy chain CDR1, (e) a heavy chain CDR2, and (f) a heavy chain CDR3 (SEQ ID NOs: 21-120).

In various embodiments of the disclosed methods, an antigen binding molecule of anti-CD20 scFv-14 comprises a heavy chain (HC), and the HC can comprise a heavy chain variable region (VH) sequence comprising one of SEQ ID NOs: 1-10. Moreover, in embodiments of the disclosed methods, an antigen binding molecule can be employed which comprises a VH amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule of claim disclosed herein (e.g., an antigen binding molecule comprising a variable region (VH) sequence comprising one of SEQ ID NOs: 1-10).

In various embodiments of the disclosed methods, an antigen binding molecule of GALV gp70 comprises a heavy chain (HC), and the HC can comprise a heavy chain variable region (VH) sequence comprising one of SEQ ID NOs: 303-314. Moreover, in embodiments of the disclosed methods, an antigen binding molecule can be employed which comprises a VH amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule of claim disclosed herein (e.g., an antigen binding molecule comprising a variable region (VH) sequence comprising one of SEQ ID NOs: 303-314).

In various embodiments of the disclosed methods, an antigen binding molecule of anti-CD20 scFv-14 comprises a light chain (LC), and the LC can comprise a light chain variable region (LH) sequence comprising one of SEQ ID NOs: 11-20. In various embodiments of the disclosed methods the light chain comprises a light chain CDR1, a light chain CDR2, and a light chain CDR3. Moreover, in embodiments of the disclosed methods, an antigen binding molecule can be employed which comprises a VL amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule of claim disclosed herein (e.g., an antigen binding molecule comprising a variable region (VL) one of a sequence comprising SEQ ID NO: 11-20).

In various embodiments of the disclosed methods, an antigen binding molecule of GALV gp70 comprises a light chain (LC), and the LC can comprise a light chain variable region (LH) sequence comprising one of SEQ ID NOs: 315-324. In various embodiments of the disclosed methods the light chain comprises a light chain CDR1, a light chain CDR2, and a light chain CDR3. Moreover, in embodiments of the disclosed methods, an antigen binding molecule can be employed which comprises a VL amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule of claim disclosed herein (e.g., an antigen binding molecule comprising a variable region (VL) one of a sequence comprising SEQ ID NO: 315-324).

In specific embodiments of the disclosed methods, a variable heavy chain comprises SEQ ID NO: 5.

In specific embodiments of the disclosed methods, a variable light chain comprises SEQ ID NO: 15.

In specific embodiments of the disclosed methods, the antigen binding molecule of anti-CD20 scFv-14 comprises a heavy chain CDR1 comprising the amino acid sequence SEQ ID NO: 25, a heavy chain CDR2 comprising the amino acid sequence SEQ ID NO: 46, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed methods, the antigen binding molecule of anti-CD20 scFv-14 comprises a heavy chain CDR1 comprising the amino acid sequence SEQ ID NO: 32, a heavy chain CDR2 comprising the amino acid sequence SEQ ID NO: 54, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed methods, the antigen binding molecule of anti-CD20 scFv-14 comprises a heavy chain CDR1 comprising the amino acid sequence SEQ ID NO: 39, a heavy chain CDR2 comprising the amino acid sequence SEQ ID NO: 62, a heavy chain CDR3 comprising the amino acid sequence SEQ ID NO: 80, a light chain CDR1 comprising the amino acid sequence SEQ ID NO: 98, a light chain CDR2 comprising the amino acid sequence SEQ ID NO: 110, and a light chain CDR3 comprising the amino acid sequence SEQ ID NO: 116.

In specific embodiments of the disclosed method, with respect to the GALV gp70 binding molecule, a heavy chain variable region of the binding molecule comprises: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY.

In specific embodiments of the disclosed method, with respect to the GALV gp70 binding molecule, a light chain variable region of the binding molecule comprises: a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments of the disclosed method, with respect to the GALV gp70 binding molecule, a light chain variable region and a heavy chain variable region of the binding molecule comprise: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY; a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In view of the above description of antigen binding molecules that can be employed in the disclosed methods, representative methods will now be discussed in more detail.

### a) Method of Determining a Number of Cells or Viral Particles Presenting a Molecule of Interest

There are situations in which it may be desirable to determine the number of cells present in a sample that are expressing a molecule of interest. For example, it may be desirable to determine the number of immune cells present a sample obtained from a subject that are expressing a molecule of interest. Or it may be desirable to determine the number of cells transfected and expressing a molecule of interest, which can be used as a measure of the level of efficiency of the transfection. The disclosed method can be employed in these and other applications in which it is desirable to determine the number of cells present in a sample that are expressing a molecule of interest.

Thus, a method of determining a number of cells presenting a molecule in a sample wherein the molecule comprises an amino acid sequence selected from the group consisting of any one or more of the amino acid sequences described in Tables 3A-3C, 4A-4C or 7A-7C, 8A-8C, 9-10 is provided.

In one embodiment, a sample comprising cells known or suspected to be expressing a molecule of interest comprising an amino acid sequence selected from the group consisting of any of the amino acid sequences described herein is provided.

In specific embodiments the selected amino acid sequence is QVQLQQSGAELMKPGASVKLSCKATGHTFTGYWIEWVKQRPGHGLEWIGEILPGSGST NYNEKFKGKATFTADTSSNTAYMQLSSLTTEDSAIYYCAREGFAYWGQGTLVTVSA (SEQ ID NO: 5); in other embodiments the selected amino acid sequence is DIVMTQSHKFMSTSVGDRVSITCKASQDVGIAVAWYQQKPGQSPKLLIYWASTRHTGV PDRFTGSGSGTDFTLTISNVQSEDLADYFCQQYSSYPYTFGGGTKLEIK (SEQ ID NO: 15); in other embodiments the selected amino acid sequence is selected from (SEQ ID NOs: 25, 46, 70, 32, 54, 39, 62, 80, 90, 98, 104, 116) or any combination.

In specific embodiments the selected amino acid sequence is

(SEQ ID NO: 325)
MVLLPGSMLLTSNLHHLRHQMSPGSWKRLIILLSCVFGGGGTSLQNKNPH
QPMTLTWQVLSQTGDVVWDTKAVQPPWTWWPTLKPDVCALAASLESWDIP
GTDVSSSKRVRPPDSDYTAAYKQITWGAIGCSYPRARTRMASSTFYVCPR
DGRTLSEARRCGGLESLYCKEWDCETTGTGYWLSKSSKDLITVKWDQNSE
WTQKFQQCHQTGWCNPLKIDFTDKGKLSKDWITGKTWGLRFYVSGHPGVQ
FTIRLKITNMPAVAVGPDLVLVEQGPPRTSLALPPPLPPREAPPPSLPDS
NSTALATSAQTPTVRKTIVTLNTPPPTTGDRLFDLVQGAFLTLNATNPGA
TESCWLCLAMGPPYYEAIASSGEVAYSTDLDRCRWGTQGKLTLTEVSGHG
LCIGKVPFTHQHLCNQTLSINSSGDHQYLLPSNHSWWACSTGLTPCLSTS
VFNQTRDFCIQVQLIPRIYYYPEEVLLQAYDNSHPRTKREAVSLTLAVLL
GLGITAGIGTGSTALIKGPIDLQQGLTSLQIAIDADLRALQDSVSKLEDS
LTSLSEVVLQNRRGLDLLFLKEGGLCAALKEECCFYIDHSGAVRDSMKKL
KEKLDKRQLERQKSQNWYEGWENNSPWFTTLLSTIAGPLLLLLLLLILGP
CIINKLVQFINDRISAVKILVLRQKYQALENEGNL.

The cell can be of any type, and can be human or non-human (e.g., mouse, rat, rabbit, hamster, etc). In a preferred embodiment, the cell is an immune cell. An immune cell of the method can be any type of immune cell (e.g., B lymphocytes, monocytes, dendritic cells, Langerhans cells, keratinocytes, endothelial cells, astrocytes, fibroblasts, and oligodendrocytes). T cells (including T cytotoxic, T helper and Treg cells) are especially preferred. In specific embodiments, the cells are T cells, which can be obtained as described herein and by methods known in the art. Any type of immune cell can be employed in this embodiment of the disclosed method. Exemplary cells include, but are not limited to immune cells such as T cells, tumor infiltrating lymphocytes (TILs), NK cells, TCR-expressing cells, dendritic cells, and NK-T cells. The T cells can be autologous, allogeneic, or heterologous. The T cells can be CD4+ T cells or CD8+ T cells. When a T cell is employed in the disclosed methods, the T cell can be an in vivo T cell or an in vitro T cell. Moreover, the cells can be disposed in, or isolated from, any environment capable of maintaining the cells in a viable form, such as blood, tissue or any other sample obtained from a subject, cell culture media, tissue grown ex vivo, a suitable buffer, etc.

In specific embodiments, the molecule of interest is a CAR. When the molecule is a CAR it can comprise a molecule, or fragment thereof, selected from the group consisting of CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, and combinations thereof.

The sample is then contacted with an antigen binding molecule that specifically binds the molecule of interest and comprises a detectable label, under conditions that permit the formation of a binding complex comprising a cell present in the sample and the antigen binding molecule. The antigen binding molecule is preferably an antigen binding molecule (or fragment thereof) disclosed herein, e.g., in the Figures, Sequence Listing or the instant section of the disclosure. Any antigen binding molecule that specifically binds all or a portion of an anti-CD20 scFv-14 molecule can be employed in the disclosed method. Multiple examples of suitable antigen binding molecules are provided herein, e.g., those having one or more of the CDRs shown in any of the Tables presented herein such as Tables 3A-3C, 4A-4C or 7A-7C, 8A-8C.

Any detectable label can be employed in the method, and suitable labels can be selected using a desired set of criteria. Examples of types of detectable labels include a fluorescent dye, which can be selected from the group consisting of an Atto dye, an Alexafluor dye, quantum dots, Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhodamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midoriishi Cyan, Wild Type GFP, GFP (S65C mutation), TurboGFP, TagGFP, GFP (S65L mutation), Emerald, GFP (S65T mutation), EGFP, Azami Green, ZsGreen1, TagYFP, EYFP, Topaz, Venus, mcitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoerythrin (BPE), mCherry, HcRed1, Katusha, P3, Peridinin Chlorophyll (PerCP), mKate (TagFP635), TurboFP635, mPlum, and mRaspberry. Other types of detectable labels include optical dyes, which are described in Johnson, Molecular Probes Handbook: A Guide to Fluorescent Probes and Labeling Techniques, 11^{th} Edition, Life Technologies, (2010), hereby expressly incorporated by reference, radiolabels (e.g., isotope markers such as ³H, ¹¹C, ¹⁴C, ¹⁵N, ¹⁸F, ³⁵S, ⁶⁴CU, ⁹⁰Y, ⁹⁹Tc, ¹¹¹In, ¹²⁴I, ¹²⁵I, ¹³¹I), photochromic compounds, magnetic labels (e.g., DYNABEADS), etc. Strategies for the labeling of proteins are known in the art and can be employed in the disclosed method.

The label can be associated with the antigen binding molecule at any position in the molecule, although it is preferable to associate the label with the molecule at a position (or positions, if multiple labels are employed) at a point such that the binding properties of the molecule are not modified (unless such modified binding activity is desired). Any antigen binding molecule or fragment thereof that specifically binds all or a part of the molecule of interest comprising an anti-CD20 scFv-14 molecule can be employed in the disclosed method. In other aspects, any antigen binding molecule or fragment thereof that specifically binds all or a part of the molecule of interest comprising GALV gp70 molecule can be employed in the disclosed method.

In specific embodiments of the disclosed method, with respect to the anti-CD20 scFv-14 molecule, the antigen binding molecule comprises a heavy chain CDR1 comprising the amino acid sequence SEQ ID NO: 25, a heavy chain CDR2 comprising the amino acid sequence SEQ ID NO: 46, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, with respect to the anti-CD20 scFv-14 molecule, the antigen binding molecule comprises a heavy chain CDR1 comprising the amino acid sequence SEQ ID NO: 32, a heavy chain CDR2 comprising the amino acid sequence SEQ ID NO: 54, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, with respect to the anti-CD20 scFv-14 molecule, the antigen binding molecule comprises a heavy chain CDR1 comprising the amino acid sequence SEQ ID NO: 39, a heavy chain CDR2 comprising the amino acid sequence SEQ ID NO: 62, a heavy chain CDR3 comprising the amino acid sequence SEQ ID NO: 80, a light chain CDR1 comprising the amino acid sequence SEQ ID NO: 98, a light chain CDR2 comprising the amino acid sequence SEQ ID NO: 110, and a light chain CDR3 comprising the amino acid sequence SEQ ID NO: 116.

In specific embodiments of the disclosed method, with respect to the GALV gp70 binding molecule, the HCDR1 has a sequence according to any one of SEQ ID NOs: 127-138, 157-168, and 187-198; the HCDR2 has a sequence according to any one of SEQ ID NOs:139-150, 169-180, and 199-210; the HCDR3 has a sequence according to any one of SEQ ID NOs:151-156, 181-186, 211-222 and DYY; the LCDR1 has a sequence according to any one of SEQ ID NOs: 223-232, 253-262, and 283-292; the LCDR2 has a sequence according to any one of SEQ ID NOs: 233-242, 263-272, SGS, GTN, RAS, DTS, and KVS; and the LCDR3 has a sequence according to any one of SEQ ID NOs: 243-252, 273-282, and 293-302.

In specific embodiments of the disclosed method, with respect to the GALV gp70 binding molecule, a heavy chain variable region of the binding molecule comprises: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY.

In specific embodiments of the disclosed method, with respect to the GALV gp70 binding molecule, a light chain variable region of the binding molecule comprises: a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments of the disclosed method, with respect to the GALV gp70 binding molecule, a light chain variable region and a heavy chain variable region of the binding molecule comprise: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY; a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

The antigen binding molecule can be disposed on any surface, or no surface at all. For example, the antigen binding molecule can be present in a buffer and the buffer-antigen binding molecule can be contacted with the sample. Alternatively, the antigen binding molecule can be associated with a surface. Suitable surfaces include agarose beads, magnetic beads such as DYNABEADS™, or a plastic, glass or ceramic plate such as a welled plate, a bag such as a cell culture bag, etc. The surface can itself be disposed in another structure, such as a column.

Conditions that permit the formation of a binding complex will be dependent on a variety of factors, however generally aqueous buffers at physiological pH and ionic strength, such as in phosphate-buffered saline (PBS), will favor formation of binding complexes and are preferred in the disclosed method.

Continuing, the number of cells present in a binding complex in the sample is determined. The specific method employed to determine the number of cells present in a binding complex will be dependent on the nature of the label selected. For example, FACS can be employed when a fluorescent label is selected; when an isotope label is selected mass spectrometry, NMR or other technique can be employed; magnetic-based cell sorting can be employed when a magnetic label is chosen; microscopy can also be employed. The output of these detection methods can be in the form of a number of cells or the output can be of a form that allows the calculation of the number of cells based on the output.

### b) Method of Determining the Presence or Absence of a Molecule

It is of value to have the ability to separate populations of different molecules, and particularly biologically-relevant molecules, from one another. Using the antigen binding molecules provided herein, such separation can be achieved and employed in a range of biotechnological, biopharmaceutical and therapeutic applications.

In one aspect of the instant disclosure, a method of isolating a molecule comprising all or a portion of an anti-CD20 scFv-14 molecule is provided. In another aspect of the instant disclosure, a method of isolating a molecule comprising a molecule comprising one or more of the amino acid sequences described in Tables 3A-3C and 4A-4C is provided.

In a further aspect of the instant disclosure, a method of isolating a molecule comprising all or a portion of a GALV gp70 molecule is provided. In another aspect of the instant disclosure, a method of isolating a molecule comprising a molecule comprising one or more of the amino acid sequences described in Tables 7A-7C and 8A-8C is provided.

In one embodiment, the method comprises providing a sample known or suspected to comprise all or a portion of an anti-CD20 scFv-14 molecule. In one embodiment, the method comprises providing a sample known or suspected to comprise all or a portion of GALV gp70 molecule.

In one embodiment, the method comprises providing a sample known or suspected to comprise one of more of the amino acid sequences of interest.

In specific embodiments the selected amino acid sequence is QVQLQQSGAELMKPGASVKLSCKATGHTFTGYWIEWVKQRPGHGLEWIGEILPGSGST NYNEKFKGKATFTADTSSNTAYMQLSSLTTEDSAIYYCAREGFAYWGQGTLVTVSA (SEQ ID NO: 5); in other embodiments the selected amino acid sequence is DIVMTQSHKFMSTSVGDRVSITCKASQDVGIAVAWYQQKPGQSPKLLIYWASTRHTGV PDRFTGSGSGTDFTLTISNVQSEDLADYFCQQYSSYPYTFGGGTKLEIK (SEQ ID NO: 15); in other embodiments the selected amino acid sequence is selected from (SEQ ID NOs: 25, 46, 70, 32, 54, 39, 62, 80, 90, 98, 104, 116) or any combination.

In specific embodiments the selected amino acid sequence is

(SEQ ID NO: 325)
MVLLPGSMLLTSNLHHLRHQMSPGSWKRLIILLSCVFGGGGTSLQNKNPH
QPMTLTWQVLSQTGDVVWDTKAVQPPWTWWPTLKPDVCALAASLESWDIP
GTDVSSSKRVRPPDSDYTAAYKQITWGAIGCSYPRARTRMASSTFYVCPR
DGRTLSEARRCGGLESLYCKEWDCETTGTGYWLSKSSKDLITVKWDQNSE
WTQKFQQCHQTGWCNPLKIDFTDKGKLSKDWITGKTWGLRFYVSGHPGVQ
FTIRLKITNMPAVAVGPDLVLVEQGPPRTSLALPPPLPPREAPPPSLPDS
NSTALATSAQTPTVRKTIVTLNTPPPTTGDRLFDLVQGAFLTLNATNPGA
TESCWLCLAMGPPYYEAIASSGEVAYSTDLDRCRWGTQGKLTLTEVSGHG
LCIGKVPFTHQHLCNQTLSINSSGDHQYLLPSNHSWWACSTGLTPCLSTS
VFNQTRDFCIQVQLIPRIYYYPEEVLLQAYDNSHPRTKREAVSLTLAVLL
GLGITAGIGTGSTALIKGPIDLQQGLTSLQIAIDADLRALQDSVSKLEDS
LTSLSEVVLQNRRGLDLLFLKEGGLCAALKEECCFYIDHSGAVRDSMKKL
KEKLDKRQLERQKSQNWYEGWENNSPWFTTLLSTIAGPLLLLLLLLILGP
CIINKLVQFINDRISAVKILVLRQKYQALENEGNL.

In specific embodiments, the molecule of interest is a CAR. When the molecule is a CAR it can comprise a molecule, or fragment thereof, selected from the group consisting of CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, and combinations thereof.

An antigen binding molecule that specifically binds all or a portion of an anti-CD20 scFv-14 molecule or GALV gp70 protein and optionally comprises a detectable label is provided. When it is decided to employ a detectable label, any detectable label can be employed in the method, as described herein, and suitable labels can be selected using a desired set of criteria. Examples of types of detectable labels include fluorescent labels (e.g., fluorescein, rhodamine, tetramethylrhodamine, eosin, erythrosin, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cas-cade Yellow and R-phycoerythrin (PE) (Molecular Probes), FITC, Rhodamine, and Texas Red (Pierce), Cy5, Cy5.5, Cy7 (Amersham Life Science)). Suitable optical dyes, including fluorophores, are described in Johnson, Molecular Probes Handbook: A Guide to Fluorescent Probes and Labeling Techniques, 11^{th} Edition, Life Technologies, (2010), hereby expressly incorporated by reference, radiolabels (e.g., isotope markers such as ³H, ¹¹C, ¹⁴C, ¹⁵N, ¹⁸F, ³⁵S, ⁶⁴CU, ⁹⁰Y, ⁹⁹Tc, ¹¹¹In, ¹²⁴I, ¹²⁵I, ¹³¹I). Photochromic compounds, a Halo-tag, Atto dyes, Tracy dyes, proteinaceous fluorescent labels (e.g., proteinaceous fluorescent labels also include, but are not limited to, green fluorescent protein, including a Renilla, Ptilosarcus, or Aequorea species of GFP (Chalfie et al., (1994) Science 263:802-805), EGFP (Clon-tech Labs., Inc., Genbank Accession Number U55762), blue fluorescent protein (BFP, Quantum Biotechnologies, Inc; Stauber, (1998) Biotechniques 24:462-471; Heim et al., (1996) Curr. Biol. 6: 178-182), enhanced yellow fluorescent protein (Clontech Labs., Inc.), luciferase (Ichiki et al., (1993) J. Immunol. 150:5408-5417), magnetic labels (e.g., DYNABEADS), etc can also be employed. Strategies for the labeling of proteins are well known in the art and can be employed in the disclosed method.

The label can be associated with the antigen binding molecule at any position in the molecule, although it is preferable to associate the label with the molecule at a position (or positions, if multiple labels are employed) at a point such that the binding properties of the molecule are not modified (unless such modified binding activity is desired). With respect to an anti-CD20 scFv-14 molecule, any antigen binding molecule, or fragment thereof, that specifically binds all or a portion of an anti-CD20 scFv-14 molecule can be employed, such as those disclosed herein, e.g., those having one or more of the CDRs shown in Tables 3A-3C and 4A-4C. With respect to a GALV gp70 protein, any antigen binding molecule, or fragment thereof, that specifically binds all or a portion of a GALV gp70 protein can be employed, such as those disclosed herein, e.g., those having one or more of the CDRs shown in Tables 7A-7C and 8A-8C.

In specific embodiments of the disclosed method, the antigen binding molecule of anti-CD20 scFv-14 comprises a heavy chain CDR1 comprising the amino acid sequence SEQ ID NO: 25, a heavy chain CDR2 comprising the amino acid sequence SEQ ID NO: 46, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 32, a heavy chain CDR2 comprising the amino acid sequence SEQ ID NO: 54, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 39, a heavy chain CDR2 comprising the amino acid sequence SEQ ID NO: 62, a heavy chain CDR3 comprising the amino acid sequence SEQ ID NO: 80, a light chain CDR1 comprising the amino acid sequence SEQ ID NO: 98, a light chain CDR2 comprising the amino acid sequence SEQ ID NO: 110, and a light chain CDR3 comprising the amino acid sequence SEQ ID NO: 116.

In specific embodiments of the disclosed method, with respect to the GALV gp70 binding molecule, a heavy chain variable region of the binding molecule comprises: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY.

In specific embodiments of the disclosed method, with respect to the GALV gp70 binding molecule, a light chain variable region of the binding molecule comprises: a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments of the disclosed method, with respect to the GALV gp70 binding molecule, a light chain variable region and a heavy chain variable region of the binding molecule comprise: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY; a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

The antigen binding molecule can be disposed on any surface, or no surface at all. For example, the antigen binding molecule can be present in a buffer and the buffer-antigen binding molecule can be contacted with the sample. Alternatively, the antigen binding molecule can be associated with a surface. Suitable surfaces include agarose beads, magnetic beads such as DYNABEADS™, or a plastic, glass or ceramic plate such as a welled plate, a bag such as a cell culture bag, etc. The surface can itself be disposed in another structure, such as a column.

The sample is contacted with the antigen binding molecule, under conditions that permit the formation of a binding complex comprising a molecule comprising the selected amino acid sequence and the antigen binding molecule. Conditions that permit the formation of a binding complex will be dependent on a variety of factors, however generally aqueous buffers at physiological pH and ionic strength, such as in phosphate-buffered saline (PBS), will favor formation of binding complexes and are preferred in the disclosed method. Since the component parts of a binding complex can be disposed on surfaces as described herein, formed binding complexes can also be disposed on surfaces.

At this stage, no binding complexes may have formed, or a plurality of binding complexes comprising one or more antigen binding molecules bound to a molecule comprising all or a portion of an anti-CD20 scFv-14 molecule or a GALV gp70 protein may have formed. Unbound molecules comprising the selected amino acid sequence and/or unbound antigen binding molecules may also be present in the local environment of any formed binding complexes.

Any molecules not part of a binding complex are then separated from any formed binding complexes. The method of the removal will depend on the structure and/or local environment of the binding complexes. For example, if the antigen binding molecule is disposed on a bead, plate or bag the unbound components of the reaction mixture can be washed away using a solution that leaves formed binding complexes intact. If a binding complex is disposed on a bead, the bead itself may be situated in a column or other structure and the same approach can be used.

The solution used to induce the formation of binding complexes can be used, for example, as a wash solution to remove unbound components. Any suitable buffer or solution that does not disrupt formed binding complexes can be used. Typically, buffers having high salt concentrations, non-physiological pH, containing chaotropes or denaturants, are preferably avoided when performing this step of the method.

A formed binding complex is then separated into (a) a molecule comprising all or a portion of an anti-CD20 scFv-14 molecule or a GALV gp70 protein, and (b) an antigen binding molecule (e.g., one or more of SEQ ID NOs. 21-120) or comprising a sequence according to one of more of SEQ ID NOs: 127-324. The separation can be achieved using standard methodologies known to those of skill in the art. For example, a solution of suitable pH and composition can be washed over the complexes. A solution that is commonly employed for this purpose is 0.1 M glycine HCl, pH 2.5-3.0, and this solution can be employed to achieve the separation. Other solutions that can be employed include 100 mM citric acid, pH 3.0, 50-100 mM triethylamine or triethanolamine, pH 11.5; 150 mM ammonium hydroxide, pH 10.5; 0.1 M glycine-NaOH, pH 10.0; 5 M lithium chloride, 3.5 M magnesium or potassium chloride, 3.0 M potassium chloride, 2.5 M sodium or potassium iodide, 0.2-3.0 M sodium thiocyanate, 0.1 M Tris-acetate with 2.0 M NaCl, pH 7.7; 2-6 M guanidine HCl, 2-8 M urea, 1.0 M ammonium thiocyanate, 1% sodium deoxycholate 1% SDS; and 10% dioxane 50% ethylene glycol, pH 8-11.5.

Following the separation, if the molecule comprising all or a portion of an anti-CD20 scFv-14 molecule or a GALV gp70 protein is of primary interest it can be collected; alternatively, if the antigen binding molecule is of primary interest it can be collected.

### c) Method of Determining the Presence or Absence of a Molecule

As disclosed herein, it may sometimes be desirable to isolate a molecule comprising all or a portion of an anti-CD20 scFv-14 molecule. In other cases, simply knowing whether a molecule comprising all or a portion of an anti-CD20 scFv-14 molecule, is present or absent from a sample is enough information. For example, it may be beneficial to know that such a molecule is being expressed, regardless of the level of expression. In other cases it may be desirable to know if a purification process or step designed to remove such a molecule has been effectively. Thus, the qualitative determination of the presence or absence of a molecule comprising all or a portion of an anti-CD20 scFv-14 molecule, can be useful in multiple applications. The amino acid sequences described in Tables 3A-3C and 4A-4C may be used in any of the quantitative or qualitative methods described herein to determine the presence, absence, and/or quantity of all or a portion of an anti-CD20 scFv-14 molecule.

As disclosed herein, it may sometimes be desirable to isolate a molecule comprising all or a portion of a GALV gp70 protein. In other cases, simply knowing whether a molecule comprising all or a portion of a GALV gp70 protein molecule, is present or absent from a sample is enough information. For example, it may be beneficial to know that such a molecule is being expressed, regardless of the level of expression. In other cases it may be desirable to know if a purification process or step designed to remove such a molecule has been effectively. Thus, the qualitative determination of the presence or absence of a molecule comprising all or a portion of a GALV gp70 protein, can be useful in multiple applications. The amino acid sequences described in Tables 7A-7C and 8A-8C may be used in any of the quantitative or qualitative methods described herein to determine the presence, absence, and/or quantity of all or a portion of a GALV gp70 protein.

In view thereof, a method of determining the presence or absence in a sample of a molecule comprising all or a portion of an anti-CD20 scFv-14 molecule or a GALV gp70 protein in a sample is provided.

In one embodiment, the method comprises providing a sample known or suspected to comprise all or a portion of an anti-CD20 scFv-14 molecule or a GALV gp70 protein.

In specific embodiments, all or a portion of an anti-CD20 scFv-14 molecule is a CAR. When the molecule is a CAR it can comprise a molecule, or fragment thereof, selected from the group consisting of CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, and combinations thereof.

An antigen binding molecule (e.g., SEQ ID NOs: 20-120) comprising a detectable label, which antigen binding molecule (e.g. all or a portion of an anti-CD20 scFv-14 molecule) specifically binds is provided. In other aspects, an antigen binding molecule (e.g., SEQ ID NOs: 127-324) comprising a detectable label, which antigen binding molecule (e.g. all or a portion of a GALV gp70 protein) specifically binds is provided. Suitable labels can be selected using a desired set of criteria. Examples of types of detectable labels include fluorescent labels (e.g., fluorescein, rhodamine, tetramethylrhodamine, eosin, erythrosin, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cas-cade Yellow and R-phycoerythrin (PE) (Molecular Probes), FITC, Rhodamine, and Texas Red (Pierce), Cy5, Cy5.5, Cy7 (Amersham Life Science)). Suitable optical dyes, including fluorophores, are described in Johnson, Molecular Probes Handbook: A Guide to Fluorescent Probes and Labeling Techniques, 11^{th} Edition, Life Technologies, (2010), hereby expressly incorporated by reference, radiolabels (e.g., isotope markers such as ³H, ¹¹C, ¹⁴C, ¹⁵N, ¹⁸F, ³⁵S, ⁶⁴CU, ⁹⁰Y, ⁹⁹Tc, ¹¹¹In, ¹²⁴I, ¹²⁵I, ¹³¹I) Photochromic compounds, a Halo-tag, Atto dyes, Tracy dyes, proteinaceous fluorescent labels (e.g., proteinaceous fluorescent labels also include, but are not limited to, green fluorescent protein, including a Renilla, Ptilosarcus, or Aequorea species of GFP (Chalfie et al., (1994) Science 263:802-805), EGFP (Clon-tech Labs., Inc., Genbank Accession Number U55762), blue fluorescent protein (BFP, Quantum Biotechnologies, Inc; Stauber, (1998) Biotechniques 24:462-471; Heim et al., (1996) Curr. Biol. 6: 178-182), enhanced yellow fluorescent protein (Clontech Labs., Inc.), luciferase (Ichiki et al., (1993) J. Immunol. 150:5408-5417), magnetic labels (e.g., DYNABEADS), etc can also be employed. Strategies for the labeling of proteins are well known in the art and can be employed in the disclosed method.

In specific embodiments of the disclosed method, the antigen binding molecule of anti-CD20 scFv-14 comprises a heavy chain CDR1 comprising the amino acid sequence SEQ ID NO: 25, a heavy chain CDR2 comprising the amino acid sequence SEQ ID NO: 46, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 32, a heavy chain CDR2 comprising the amino acid sequence SEQ ID NO: 54, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 39, a heavy chain CDR2 comprising the amino acid sequence SEQ ID NO: 62, a heavy chain CDR3 comprising the amino acid sequence SEQ ID NO: 80, a light chain CDR1 comprising the amino acid sequence SEQ ID NO: 98, a light chain CDR2 comprising the amino acid sequence SEQ ID NO: 110, and a light chain CDR3 comprising the amino acid sequence SEQ ID NO: 116.

In specific embodiments of the disclosed method, with respect to the GALV gp70 binding molecule, a heavy chain variable region of the binding molecule comprises: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY.

In specific embodiments of the disclosed method, with respect to the GALV gp70 binding molecule, a light chain variable region of the binding molecule comprises: a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments of the disclosed method, with respect to the GALV gp70 binding molecule, a light chain variable region and a heavy chain variable region of the binding molecule comprise: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY; a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

The label can be associated with the antigen binding molecule at any position in the molecule, although it is preferable to associate the label with the molecule at a position (or positions, if multiple labels are employed) at a point such that the binding properties of the molecule are not modified (unless such modified binding activity is desired). Any antigen binding molecule that specifically binds all or a portion of an anti-CD20 scFv-14 molecule or a GALV gp70 protein can be employed, such as those disclosed herein, e.g., those having one or more of the CDRs shown in Tables 3A-3C, 4A-4C or 7A-7C, 8A-8C.

Continuing, the sample is contacted with the antigen binding molecule under conditions that permit the formation of a binding complex comprising a cell present in the sample and the antigen binding molecule. The antigen binding molecule can be disposed on any surface, or no surface at all. For example, the antigen binding molecule can be present in a buffer and the buffer-antigen binding molecule can be contacted with the sample. Alternatively, the antigen binding molecule can be associated with a surface. Suitable surfaces include agarose beads, magnetic beads such as DYNABEADS™, or a plastic, glass or ceramic plate such as a welled plate, a bag such as a cell culture bag, etc. The surface can itself be disposed in another structure, such as a column.

The sample is contacted with the antigen binding molecule, under conditions that permit the formation of a binding complex comprising a molecule comprising the molecule comprising all or a portion of an anti-CD20 scFv-14 molecule or GALV gp70 protein and the antigen binding molecule. Conditions that permit the formation of a binding complex will be dependent on a variety of factors, however generally aqueous buffers at physiological pH and ionic strength, such as in phosphate-buffered saline (PBS), will favor formation of binding complexes and are preferred in the disclosed method. Since the component parts of a binding complex can be disposed on surfaces as described herein, formed binding complexes can also be disposed on surfaces.

At this stage, no binding complexes may have formed, or a plurality of binding complexes comprising one or more antigen binding molecules bound to a molecule comprising all or a portion of an anti-CD20 scFv-14 molecule (or one or more molecules comprising the all or a portion of an anti-CD20 scFv-14 molecule bound to an antigen binding molecule [e.g., SEQ ID NOs: 1-120]) may have formed. Unbound molecules comprising the all or a portion of an anti-CD20 scFv-14 molecule and/or unbound antigen binding molecules may also be present in the local environment of any formed binding complexes.

In other aspects, at this stage, no binding complexes may have formed, or a plurality of binding complexes comprising one or more antigen binding molecules bound to a molecule comprising all or a portion of a GALV gp70 protein may have formed. Unbound molecules comprising the all or a portion of a GALV gp70 protein and/or unbound antigen binding molecules may also be present in the local environment of any formed binding complexes.

Any molecules not part of a binding complex are then separated from any formed binding complexes. The method of the removal will depend on the structure and/or local environment of the binding complexes. For example, if the antigen binding molecule is disposed on a bead, plate or bag the unbound components of the reaction mixture can be washed away using a solution that leaves formed binding complexes intact. If a binding complex is disposed on a bead, the bead itself may be situated in a column or other structure and the same approach can be used.

The solution used to induce the formation of binding complexes can be used, for example, as a wash solution to remove unbound components. Any suitable buffer or solution that does not disrupt formed binding complexes can also be used. Typically, buffers having high salt concentrations, non-physiological pH, containing chaotropes or denaturants, should be avoided when performing this step of the method.

Lastly, the presence or absence of a binding complex, which will comprise a molecule comprising all or a portion of an anti-CD20 scFv-14 molecule or GALV gp70 protein binding molecule (e.g., one or more described in Tables 3A-3C, 4A-4C or 7A-7C, 8A-8C, 9-10) is detected. The specific method employed to detect the presence or absence of a binding complex will be dependent on the nature of the label selected. For example, FACS can be employed when a fluorescent label is selected; when an isotope label is selected mass spectrometry, NMR or other technique can be employed; magnetic-based cell sorting can be employed when a magnetic label is chosen; microscopy can also be employed. The end result of the method is a qualitative assessment of the presence or absence of the antigen binding molecule comprising the detectable label, and thus, the presence or absence of its binding partner, the molecule comprising all or a portion of an anti-CD20 scFv-14 molecule or GALV gp70 protein.

As is the case with all of the disclosed methods, the molecule comprising all or a portion of an anti-CD20 scFv-14 molecule or GALV gp70 protein can be disposed in any environment. In preferred embodiments, the molecule comprising all or a portion of an anti-CD20 scFv-14 molecule or GALV gp70 protein is expressed on the surface of a cell. In this embodiment, the cell can be of any type, and can be human or non-human (e.g., mouse, rate, rabbit, hamster, etc). In a preferred embodiment, the cell is an immune cell. An immune cell of the method can be any type of immune cell (e.g., B lymphocytes, monocytes, dendritic cells, Langerhans cells, keratinocytes, endothelial cells, astrocytes, fibroblasts, and oligodendrocytes). T cells (including T cytotoxic, T helper and Treg cells) are especially preferred. In specific embodiments, the cells are T cells, which can be obtained as described herein and by methods known in the art. Any type of immune cell can be employed in this embodiment of the disclosed method, and the cell can be a human or non-human cell. Exemplary cells include, but are not limited to immune cells such as T cells, tumor infiltrating lymphocytes (TILs), NK cells, TCR-expressing cells, dendritic cells, and NK-T cells. The T cells can be autologous, allogeneic, or heterologous. In additional embodiments, the cells are T cells presenting a CAR. The T cells can be CD4+ T cells or CD8+ T cells. When a T cell is employed in the disclosed methods, the T cell can be an in vivo T cell or an in vitro T cell.

In additional embodiment, the cell can be disposed in, or isolated from, any environment capable of maintaining the cell in a viable form, such as blood, tissue or any other sample obtained from a subject, cell culture media, tissue grown ex vivo, a suitable buffer, etc.

### d) Method of Increasing the Concentration of a Molecule

Very often a molecule of interest is present in a sample in lower-than-desired levels. For example, when a cell is transfected with a foreign gene expression levels of the protein(s) encoded by the foreign gene are low. The same can be true for molecules secreted from a cell; such molecules are often present in low quantities but can still be detected using the methods provided herein, if the molecule comprises one of all or a portion of an anti-CD20 scFv-14 molecule and/or one of more of the molecules described in Tables 3A-3C and 4A-4C. The same can also be true for viral particles; such viral particles are often present in low quantities but can still be detected using the methods provided herein, if the viral particles comprises all or a portion of a GALV gp70 protein. One solution to the problem of low expression levels is to increase the concentration of the molecule of interest, which can be free in solution, or expressed on the surface of a cell. The concentration of intracellularly-expressed molecules of interest can also be enhanced, however the cells must first be lysed to release the molecule.

To address this problem, a method of increasing the concentration of cells presenting a molecule comprising all or a portion of an anti-CD20 scFv-14 molecule and/or one or more molecules described in Tables 3A-3C and 4A-4C. In a further aspect, a method of increasing the concentration of viral particles presenting a molecule comprising all or a portion of a GALV gp70 protein and/or one or more molecules is described herein.

In one embodiment, the method comprises providing a sample comprising cells known or suspected to comprise a molecule comprising an amino acid sequence selected from the group consisting of all or a portion of an anti-CD20 scFv-14 molecule and/or one or more molecules described in Tables 3A-3C and 4A-4C. In a further embodiment, the method comprises providing a sample comprising cells known or suspected to comprise a molecule comprising an amino acid sequence selected from the group consisting of all or a portion of an GALV gp70 protein molecule and/or one or more molecules described in Tables 7A-7C and 8A-8C.

In specific embodiments of the disclosed method, the antigen binding molecule of anti-CD20 scFv-14 comprises a heavy chain CDR1 comprising the amino acid sequence SEQ ID NO: 25, a heavy chain CDR2 comprising the amino acid sequence SEQ ID NO: 46, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 32, a heavy chain CDR2 comprising the amino acid sequence SEQ ID NO: 54, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 39, a heavy chain CDR2 comprising the amino acid sequence SEQ ID NO: 62, a heavy chain CDR3 comprising the amino acid sequence SEQ ID NO: 80, a light chain CDR1 comprising the amino acid sequence SEQ ID NO: 98, a light chain CDR2 comprising the amino acid sequence SEQ ID NO: 110, and a light chain CDR3 comprising the amino acid sequence SEQ ID NO: 116.

In specific embodiments of the disclosed method, with respect to the GALV gp70 binding molecule, a heavy chain variable region of the binding molecule comprises: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY.

In specific embodiments of the disclosed method, with respect to the GALV gp70 binding molecule, a light chain variable region of the binding molecule comprises: a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments of the disclosed method, with respect to the GALV gp70 binding molecule, a light chain variable region and a heavy chain variable region of the binding molecule comprise: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY; a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments, the molecule comprising all or a portion of an anti-CD20 scFv-14 molecule and/or one or more molecules described in Tables 3A-3C and 4A-4C is a CAR. When the molecule is a CAR it can comprise a molecule, or fragment thereof, selected from the group consisting of CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, and combinations thereof.

An antigen binding molecule (e.g., one or more of the molecules described in Tables 3A-3C and 4A-4C) that specifically binds all or a portion of an anti-CD20 scFv-14 molecule or an antigen binding molecule that binds specifically to a GALV gp70 protein and optionally comprises a detectable label is provided. When it is preferable to employ a detectable label, any detectable label can be employed in the method, as described herein, and suitable labels can be selected using a desired set of criteria. Examples of types of detectable labels include fluorescent labels (e.g., fluorescein, rhodamine, tetramethylrhodamine, eosin, erythrosin, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cas-cade Yellow and R-phycoerythrin (PE) (Molecular Probes), FITC, Rhodamine, and Texas Red (Pierce), Cy5, Cy5.5, Cy7 (Amersham Life Science)). Suitable optical dyes, including fluorophores, are described in Johnson, Molecular Probes Handbook: A Guide to Fluorescent Probes and Labeling Techniques, 11^{th} Edition, Life Technologies, (2010), hereby expressly incorporated by reference, radiolabels (e.g., isotope markers such as ³H, ¹¹C, ¹⁴C, ¹⁵N, ¹⁸F, ³⁵S, ⁶⁴CU, ⁹⁰Y, ⁹⁹Tc, ¹¹¹In, ¹²⁴I, ¹²⁵I, ¹³¹I) Photochromic compounds, a Halo-tag, Atto dyes, Tracy dyes, proteinaceous fluorescent labels (e.g., proteinaceous fluorescent labels also include, but are not limited to, green fluorescent protein, including a Renilla, Ptilosarcus, or Aequorea species of GFP (Chalfie et al., (1994) Science 263:802-805), EGFP (Clon-tech Labs., Inc., Genbank Accession Number U55762), blue fluorescent protein (BFP, Quantum Biotechnologies, Inc; Stauber, (1998) Biotechniques 24:462-471; Heim et al., (1996) Curr. Biol. 6: 178-182), enhanced yellow fluorescent protein (Clontech Labs., Inc.), luciferase (Ichiki et al., (1993) J. Immunol. 150:5408-5417), magnetic labels (e.g., DYNABEADS), etc can also be employed. Strategies for the labeling of proteins are well known in the art and can be employed in the disclosed method.

The label can be associated with the antigen binding molecule at any position in the molecule, although it is preferable to associate the label with the molecule at a position (or positions, if multiple labels are employed) at a point such that the binding properties of the molecule are not modified (unless such modified binding activity is desired). With respect to an anti-CD20 scFv-14 molecule, any antigen binding molecule (e.g., one or more of the molecules described in Tables 3A-3C and 4A-4C) that specifically binds the molecule comprising the all or a portion of an anti-CD20 scFv-14 molecule; or one or more molecules comprising the all or a portion of an anti-CD20 scFv-14 molecule bound to an antigen binding molecule or fragment thereof) can be employed, such as those disclosed herein, e.g., those having one or more of the CDRs described in Tables 3A-3C and 4A-4C. With respect to a GALV gp70 protein, any antigen binding molecule, or fragment thereof, that specifically binds all or a portion of a GALV gp70 protein can be employed, such as those disclosed herein, e.g., those having one or more of the CDRs shown in Tables 7A-7C and 8A-8C.

In specific embodiments of the disclosed method, the antigen binding molecule of anti-CD20 scFv-14 comprises a heavy chain CDR1 comprising the amino acid sequence SEQ ID NO: 25, a heavy chain CDR2 comprising the amino acid sequence SEQ ID NO: 46, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 32, a heavy chain CDR2 comprising the amino acid sequence SEQ ID NO: 54, a heavy chain CDR3 comprising the amino acid sequence SEQ ID NO: 70, a light chain CDR1 comprising the amino acid sequence SEQ ID NO: 90, a light chain CDR2 comprising the amino acid sequence SEQ ID NO: 104, and a light chain CDR3 comprising the amino acid sequence SEQ ID NO: 116.

In specific embodiments of the disclosed method, the antigen binding molecule of anti-CD20 scFv-14 comprises a heavy chain CDR1 comprising the amino acid sequence SEQ ID NO: 39, a heavy chain CDR2 comprising the amino acid sequence SEQ ID NO: 62, a heavy chain CDR3 comprising the amino acid sequence SEQ ID NO: 80, a light chain CDR1 comprising the amino acid sequence SEQ ID NO: 98, a light chain CDR2 comprising the amino acid sequence SEQ ID NO: 110, and a light chain CDR3 comprising the amino acid sequence SEQ ID NO: 116.

In specific embodiments of the disclosed method, with respect to the GALV gp70 binding molecule, a heavy chain variable region of the binding molecule comprises: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY.

In specific embodiments of the disclosed method, with respect to the GALV gp70 binding molecule, a light chain variable region of the binding molecule comprises: a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

In specific embodiments of the disclosed method, with respect to the GALV gp70 binding molecule, a light chain variable region and a heavy chain variable region of the binding molecule comprise: a HCDR1 according to any of SEQ ID NOs: 127, 157, and 187; a HCDR2 according to any of SEQ ID NOs: 139, 169, and 199; a HCDR3 according to SEQ ID NO: 211 or DYY; a LCDR1 according to any of SEQ ID NOs: 232, 262, and 292; a LCDR2 according to any of SEQ ID NOs: 242, 272 and GTN; a LCDR3 according to any one of SEQ ID NOs: 252, 282, and 302; a HCDR1 according to any of SEQ ID NOs: 128, 158 and 188; a HCDR2 according to any of SEQ ID NOs: 140, 170, and 200; a HCDR3 according to any one of SEQ ID NOs: 151, 181, and 212; a LCDR1 according to any of SEQ ID NOs: 228, 258, and 288; a LCDR2 according to any of SEQ ID NOs: 238, 268, and KVS; a LCDR3 according to any one of SEQ ID NOs: 248, 278, and 298; a HCDR1 according to any of SEQ ID NOs: 129, 159, and 189; a HCDR2 according to any of SEQ ID NOs: 141, 171, and 201; a HCDR3 according to any one of SEQ ID NOs: 152, 182, and 213; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 130, 160, and 190; a HCDR2 according to any of SEQ ID NOs: 142, 172, and 202; a HCDR3 according to SEQ ID NO: 214 or DYY; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 131, 161, and 191; a HCDR2 according to any of SEQ ID NOs: 143, 173, and 203; a HCDR3 according to any one of SEQ ID NOs: 153, 183, and 215; a LCDR1 according to any of SEQ ID NOs: 227, 257, and 287; a LCDR2 according to any of SEQ ID NOs: 237, 267, and GTN; a LCDR3 according to any one of SEQ ID NOs: 247, 277, and 297; a HCDR1 according to any of SEQ ID NOs: 132, 162 and 192; a HCDR2 according to any of SEQ ID Nos: 144, 174 and 204; a HCDR3 according to any one of SEQ ID NOs: 154, 184, and 216; a LCDR1 according to any of SEQ ID NOs: 226, 256, and 286; a LCDR2 according to any of SEQ ID NOs: 236, 266 and DTS; a LCDR3 according to any one of SEQ ID NOs: 246, 276, and 296; a HCDR1 according to any of SEQ ID NOs: 133, 163, and 193; a HCDR2 according to any of SEQ ID NOs: 145, 175, and 205; a HCDR3 according to any one of SEQ ID NOs: 155, 185, and 217; a LCDR1 according to any of SEQ ID NOs: 225, 255, and 285; a LCDR2 according to any of SEQ ID NOs: 235, 265, and RAS; a LCDR3 according to any one of SEQ ID NOs: 245, 275, and 295; a HCDR1 according to any of SEQ ID NOs: 134, 164, and 194; a HCDR2 according to any of SEQ ID NOs: 146, 176, and 206; a HCDR3 according to SEQ ID NO: 218 or DYY; a LCDR1 according to any of SEQ ID NOs: 224, 254, and 284; a LCDR2 according to any of SEQ ID NOs: 234, 264, and GTN; a LCDR3 according to any one of SEQ ID NOs: 244, 274, and 294; a HCDR1 according to any of SEQ ID NOs: 135, 165, and 195; a HCDR2 according to any of SEQ ID NOs: 147, 177, and 207; a HCDR3 according to any one of SEQ ID NOs: 156, 186 and 219; a LCDR1 according to any of SEQ ID NOs: 223, 253, and 283; a LCDR2 according to any of SEQ ID NOs: 233, 263, and SGS; a LCDR3 according to any one of SEQ ID NOs: 243, 273, and 293; a HCDR1 according to any one of SEQ ID NOs: 136, 166, and 196; a HCDR2 according to any of SEQ ID NOs: 148, 178, and 208; a HCDR3 according to SEQ ID NO: 220 or DYY; a LCDR1 according to any of SEQ ID NOs: 229, 259, and 289; a LCDR2 according to any of SEQ ID NOs: 239, 269, and GTN; a LCDR3 according to any one of SEQ ID NOs: 249, 279, and 299; a HCDR1 according to any one of SEQ ID NOs: 137, 167, and 197; a HCDR2 according to any of SEQ ID NOs: 149, 179, and 209; a HCDR3 according to SEQ ID NO: 221 or DYY; a LCDR1 according to any of SEQ ID NOs: 231, 261 and 291; a LCDR2 according to any of SEQ ID NOs: 241, 271, and GTN; a LCDR3 according to any one of SEQ ID NOs: 251, 281, and 301; or a HCDR1 according to any one of SEQ ID NOs: 138, 168, and 198; a HCDR2 according to any of SEQ ID NOs: 150, 180, and 210; a HCDR3 according to SEQ ID NO: 222 or DYY; a LCDR1 according to any of SEQ ID NOs: 230, 260, 290; a LCDR2 according to any of SEQ ID NOs: 240, 270, and GTN; a LCDR3 according to any one of SEQ ID NOs: 250, 280, and 300.

The antigen binding molecule (e.g., one or more molecules described in Tables 3A-3C, 4A-4C, 7A-7C, 8A-8C) can be disposed on any surface, or no surface at all. For example, the antigen binding molecule can be present in a buffer and the buffer-antigen binding molecule can be contacted with the sample. Alternatively, the antigen binding molecule can be associated with a surface. Suitable surfaces include agarose beads, magnetic beads such as DYNABEADS™, or a plastic, glass or ceramic plate such as a welled plate, a bag such as a cell culture bag, etc. The surface can itself be disposed in another structure, such as a column.

A cell expressing a molecule comprising all or a portion of an anti-CD20 scFv-14 molecule can be of any type, and can be human or non-human (e.g., mouse, rate, rabbit, hamster, etc). In a preferred embodiment, the cell is an immune cell. An immune cell of the method can be any type of immune cell (e.g., B lymphocytes, monocytes, dendritic cells, Langerhans cells, keratinocytes, endothelial cells, astrocytes, fibroblasts, and oligodendrocytes). T cells (including T cytotoxic, T helper and Treg cells) are especially preferred. In specific embodiments, the cells are T cells, which can be obtained as described herein and by methods known in the art. Any type of immune cell can be employed, and the cell can be a human or non-human cell. Exemplary cells include, but are not limited to immune cells such as T cells, tumor infiltrating lymphocytes (TILs), NK cells, TCR-expressing cells, dendritic cells, and NK-T cells. The T cells can be autologous, allogeneic, or heterologous. In additional embodiments, the cells are T cells presenting a CAR. The T cells can be CD4+ T cells or CD8+ T cells. When a T cell is employed in the disclosed methods, the T cell can be an in vivo T cell or an in vitro T cell. Moreover, the cells can be disposed in, or isolated from, any environment capable of maintaining the cells in a viable form, such as blood, tissue or any other sample obtained from a subject, cell culture media, tissue grown ex vivo, a suitable buffer, etc.

The sample comprising cells and/or viral particles is contacted with the antigen binding molecule, under conditions that permit the formation of a binding complex comprising all or a portion of an anti-CD20 scFv-14 molecule or GALV gp70 protein and the antigen binding molecule (e.g., one or more molecules described in Tables 3A-3C, 4A-4C, 7A-7C, or 8A-8C). Conditions that permit the formation of a binding complex will be dependent on a variety of factors, however generally aqueous buffers at physiological pH and ionic strength, such as in phosphate-buffered saline (PBS), will favor formation of binding complexes and are preferred in the disclosed method. Since the component parts of a binding complex can be disposed on surfaces as described herein, formed binding complexes can also be disposed on surfaces.

At this stage, no binding complexes may have formed, or a plurality of binding complexes comprising one or more antigen binding molecules (e.g., one or more molecules described in Tables 3A-3C, 4A-4C, 7A-7C, 8A-8C) bound to all or a portion of an anti-CD20 scFv-14 molecule or GALV gp70 protein may have formed. Unbound molecules comprising all or a portion of an anti-CD20 scFv-14 molecule, GALV gp70 protein and/or unbound antigen binding molecules (e.g., one or more molecules described in Tables 3A-3C, 4A-4C, 7A-7C, 8A-8C) may also be present in the local environment of any formed binding complexes.

Any molecules or cells not part of a binding complex are then separated from any formed binding complexes. The method of the removal will depend on the structure and/or local environment of the binding complexes. For example, if the antigen binding molecule is bound on a bead, plate or bag the unbound components of the reaction mixture can be washed away using a solution that leaves formed binding complexes intact. If a binding complex is bound on a bead, the bead itself may be situated in a column or other structure and the same approach can be used.

The solution used to induce the formation of binding complexes can be used, for example, as a wash solution to remove unbound components. Any suitable buffer or solution that does not disrupt formed binding complexes can also be used. Typically, buffers having high salt concentrations, non-physiological pH, containing chaotropes or denaturants, should be avoided when performing this step of the method.

At this stage of the method, a population of cells presenting a molecule comprising the all or a portion of an anti-CD20 scFv-14 molecule or viral particles presenting GALV gp70 protein will be present. If a detectable label was employed, the concentration of the cells or viral particles can be easily determined, consistent with the nature of the label. Cells or viral particles not expressing the molecule comprising the all or a portion of an anti-CD20 scFv-14 molecule or GALV gp70 protein will be absent, and thus the population (or concentration) of cells presenting a molecule comprising the all or a portion of an anti-CD20 scFv-14 molecule or viral particles presenting GALV gp70 protein will be increased compared to the levels prior to performing the method.

If the concentration of the molecule comprising the all or a portion of an anti-CD20 scFv-14 molecule and/or one or more molecules described in Tables 3A-3C, 4A-4C or viral particles presenting GALV gp70 protein and/or one or more molecules described in Tables 7A-7C and 8A-8C is not at a desired level, the above steps can be repeated a desired number of times. In the context of this step of the method, a desired number of times can also be zero, if the desired concentration of cells is already present.

### EXAMPLES

Hyperimmune mice were immunized with scFv14 fused to a fragment crystallizable (Fc) domain derived from a mouse immunoglobulin protein, isotype G2a. These culture supernatants were screened for the presence of antibodies that showed specific binding to scFv14. Antibody heavy and light chain gene sequences (e.g., SEQ ID NOs: 1-20) from hybridomas selected from this screen were used to generate antibodies that were again checked for specificity to scFv14. Selected antibodies from this screen were coupled to phycoerythrin (PE) and fluorescein isothiocyanate (FITC) fluorophores and characterized by flow cytometry.

Of the 25 hybridoma supernatants, 14 showed specific binding to human CAR T cells expressing scFv14, an anti-CD20 targeting scFv, but not to irrelevant anti-CD20 CAR scFvs, an FMC63-bearing anti-CD19 CAR, or non-transduced T cells from the same healthy donor. 10 of the 14 clones were found to be suitable for antibody production. After antibody production all 10 clones were re-tested against scFv14 and unrelated anti-CD20 CARs and the FMC63-bearing anti-CD19 CAR for selectivity and sensitivity. Of these, 5 were coupled to fluorophores for characterization by flow cytometry. One of these, clone 24C12, showed robust, sensitive, and specific binding to scFv14, but not to unrelated anti-CD20 CARs or FMC63-bearing anti-CD19 CAR, and was selected for use as a flow cytometry reagent to characterize scFv14 expression in anti-CD20 samples.

An antibody generation and characterization campaign yielded antibody clone 24C12, which binds strongly to the anti-CD20 scFv contained in anti-CD20 CARs but does not bind to irrelevant anti-CD20 CARs or to anti-CD19 CAR scFvs, regardless of conjugation to either PE or FITC. The below example sections here detail the related methods.

Reagents used in this study included FACS stain buffer, Goat anti-mouse IgG AF-488, Mouse IgG isotype control, Whitlow Linker Control (LC) PE, LC AF-647, Live/Dead fixable violet stain, Live/Dead fixable aqua stain, 24C12 PE, and 24C12 FITC Abbreviations: AF, Alexa Fluor; FACS, fluorescence activated cell sorter; FITC, fluorescein isothiocyanate, IgG, immunoglobulin G; PE, phycoerythrin. LC is a custom-made antibody that binds the peptide linker between the light and heavy chains of the chimeric antigen receptor (CAR) single-chain variable fragments (scFv), enabling assessment of total CAR transduction efficiency. Abbreviations: AF, Alexa Fluor; FACS, fluorescence activated cell sorter; FITC, fluorescein isothiocyanate, IgG, immunoglobulin G; PE, phycoerythrin. KIP-1 is a custom-made antibody that binds the peptide linker between the light and heavy chains of the chimeric antigen receptor (CAR) single-chain variable fragments (scFv), enabling assessment of total CAR transduction efficiency. Equipment used in this study included Sorvall Legend XTR Centrifuge, Vi-Cell XR, and FACs Fortessa X-20 II. Abbreviations: FACS, fluorescence activated cell sorter.

These studies used an anti-CD19 CAR and three anti-CD20 CARs (Table 14). The anti-CD19 CAR, FMC63-28z, included an FMC63 anti-CD19 targeting scFv. The three anti-CD20 CARs differed only in the scFv used to target CD20; the scFvs included scFv2, scFv14, and Leu16. scFv14 and scFv2 were fully human anti-CD20 scFvs that differ in their complementarity determining regions. Leu16 is a murine anti-human CD20 scFv. See, e.g., Wu et al., (2001) Protein Eng. 2001; 14 (12):1025-33, which is incorporated herein by reference for any purpose in its entirety. Sequence analysis with IgBLAST (Ig Basic Local Alignment Search Tool) for Leu16 and FMC63 germline gene identification was completed.

TABLE 14
CAR scFvs Used in this Study

| CAR scFv domain | Vl Gene | Vh Gene | Target |
|---|---|---|---|
| scFv2 | Human Vk1-39 | Human Vh1-18 | CD20 |
| scFv14 | Human Vk1-39 | Human Vh4-39 | CD20 |
| Leu16 | Murine Vk4-72 | Murine Vh1-12 | CD20 |
| FMC63 | Murine Vk10-96 | Murine Vh2-6 | CD19 |

Abbreviations: CAR, chimeric antigen receptor; scFv, single chain variable fragment Vh; variable heavy chain; Vl, variable light chain; Vk, variable region gene segments.

### I. Example 1: Hybridoma Generation

Abveris DiversimAb™ hyperimmune mice (Canton, MA) were immunized with an scFv-Fc protein derived from scFv14 and a murine IgG2a Fc. Hybridoma supernatants were tested first in a dilution series for sensitivity to an scFv14-bearing CAR and, as a negative control, to an FMC63-bearing CAR, then tested in selectivity screens against CARs containing scFv2, scFv14, and Leu16. Select binders to scFv14, but not to FMC63, scFv2, or Leu16 underwent antibody sequencing and recombinant protein production. Purified recombinant antibodies were tested again to confirm specificity to scFv14 and subsequently conjugated to fluorophores. Conjugated antibodies were rescreened for specificity and sensitivity and one clone was selected for use in detecting scFv14.

### II. Example 2: Hybridoma Sensitivity Screening

To test the sensitivity of the hybridoma supernatants, a dilution series screen was performed. Healthy donor T cells that were either non-transduced (NTD) or transduced with an scFv 14-bearing CAR or an FMC63-bearing CAR were incubated with hybridoma supernatants and serially diluted with stain buffer. Negative controls consisted of an immunoglobulin (Ig) isotype control, conditioned media from an unrelated hybridoma, and pooled sera from mice pre-immunization. Polyclonal sera from mouse immunizations were used as a positive control.

Cells were incubated with the diluted supernatants for 45 minutes at room temperature (RT), then harvested and washed twice with stain buffer. Samples were stained. Supernatant and control samples were stained with goat anti-mouse IgG coupled to Alexa Fluor (AF)-488 (1:4,000). Controls to determine overall CAR expression were stained with the anti-linker specific antibody LC conjugated to phycoerythrin (PE) (1:1,000). All samples were incubated in stain buffer containing the viability dye Live-Dead Fixable Violet (1:2,000). The cells were stained for 45 minutes at RT, harvested, and washed twice with stain buffer then read immediately on a BD Fortessa™ flow cytometer. Data was analyzed using FlowJo™ software (BD, version 10.6) and events were systematically gated on cells (using forward scatter [FSC]-area by side scatter [SSC]-area plot), single cells (using FSC-area by FSC-height plot), live cells (viability dye), and either phycoerythrin (PE) (for the CAR control antibody) or AF-488 (for the supernatant samples) where the gating threshold was set based on the NTD control cells.

### III. Example 3: Hybridoma Specificity Testing

For specificity testing, human T cells transduced with CARs bearing either scFv2, scFv14, or Leu16 scFv were used. To ensure robust staining in the specificity screen, supernatants showing binding to scFv14 were tested at the highest concentration used in the dilution screen. Cells were stained and analyzed.

### IV. Example 4: Recombinant Antibody Characterization

After sequencing of the antibody variable heavy chain (VH) and variable light chain (VL) domains from the hybridomas chosen from the dilution series and specificity screens, antibodies were manufactured by Genscript (Piscataway, NJ) following standard procedures. Briefly, protein was expressed using Expi293F cells and one-step affinity purified using MabSelect SuRe LX (GE Healthcare, Catalog number 17-5474-02). Purity was assessed by SDS-PAGE and SEC-HPLC. Antibodies were sterile-filtered with 0.22 m filters, packaged aseptically, and stored at −80° C.

For recombinant antibody specificity and selection studies, healthy human donor T cells transduced with CARs bearing either scFv2, scFv 14, or FMC63 were harvested, washed twice with stain buffer then incubated with each of the anti-scFv14 antibodies or a mouse IgG isotype control, both at 300 ng/mL, or in the absence of primary antibodies as a negative control. NTD healthy donor T cell controls were also included. Cells were stained and analyzed.

### V. Example 5: Fluorophore Conjugation to Recombinant Antibodies

The anti-scFv14 antibody, 24C12, was sent to BD Pharmingen™ for custom conjugation to fluorophores PE and fluorescein isothiocyanate (FITC). Briefly, a heterobifunctional crosslinking reagent, succinimidyl 4-(N-maleimidomethyl) cyclohexane-1-carboxylate (SMCC), was coupled to PE and the SMCC-PE was covalently conjugated to reduced antibody. Subsequently, 1:1 PE-conjugated antibody was purified, and buffer exchanged into PBS, using size exclusion chromatography. Reactive FITC molecules were coupled to EV-aFMC63 following standard protocols to couple the fluorophore to primary amines on the antibody. Nonconjugated fluorophore was removed and the buffer was exchanged into PBS, pH 7.4, using standard size exclusion chromatography methods.

### VI. Example 6: Fluorophore-Conjugated Antibody Characterization

For characterization of the fluorophore-conjugated 24C12 antibody, donor T cells transduced with CARs bearing either scFv2, scFv 14, or FMC63 were harvested, washed twice with stain buffer then incubated with serial dilutions of 24C12 labeled with either PE or FJTC. NTD T cells from the same healthy donor, isotype controls, and unstained controls were included. All samples were incubated in stain buffer containing the viability dye Live-Dead Fixable Aqua (1:1,000). The cells were stained for 15 minutes at 4° C., harvested, washed twice with stain buffer then fixed in 0.6% (v/v) paraformaldehyde for 10 minutes at RT and stored at 4° C. until read on the BD Fortessa™ flow cytometer. Data was analyzed using FlowJo™ software (BD, version 10.6) and events were systematically gated on cells (using forward scatter [FSC]-area by side scatter [SSC]-area plot), single cells (using FSC-area by FSC-height plot), live cells (viability dye), and either PE, FITC, or AF-647 where the gating threshold was set based on the NTD control cells.

### VI. Example 7: Hybridoma Supernatant Characteristics

A panel of the 25 cryopreserved hybridomas and associated culture supernatants (study report KIT19025-1) were sent to Kite for screening (Table 15). Supernatants were characterized according to the methods described herein. Based on selection criteria herein, hybridomas (Table 15) were sequenced and produced as recombinant antibodies and characterized.

TABLE 15
Hybridoma Supernatant Characteristics

| Hybridoma Identity | Concentration (μg/mL) | KD (M) |
|---|---|---|
| 23D4 | 21.1 | 1.61E × 10¹² |
| 24C12 | under detection limit | 1.38E × 10¹² |
| 29F8 | 20.9 | 1.31E × 10¹² |
| 25F10 | 22.6 | 4.08E × 10¹² |
| 29F1 | 13.8 | 5.34E × 10¹¹ |
| 25B2 | under detection limit | 3.82E × 10¹⁰ |
| 21B5 | 13.3 | 8.23E × 10⁰⁹ |
| 22H5 | 6.6 | 6.33E × 10¹⁰ |
| 26G4 | 33.2 | 1.66E × 10⁰⁸ |
| 20C4 | 36.1 | 1.02E × 10⁰⁸ |
| 24H1 | 33.2 | 3.12E × 10⁰⁸ |
| 13C2 | 28.6 | 2.41E × 10⁰⁹ |
| 24D9 | 10.2 | 8.80E × 10⁰⁹ |
| 27B9 | 17.1 | 2.87E × 10⁰⁸ |
| 23C11 | 2.9 | 3.72E × 10⁰⁹ |
| 23E1 | 34.2 | 3.28E × 10⁰⁹ |
| 24C7 | 4.3 | 3.90E × 10⁰⁹ |
| 26E1 | 16.9 | 5.54E × 10⁰⁹ |
| 23G3 | 31.4 | 1.04E × 10⁰⁷ |
| 23A9 | 22.9 | 1.78E × 10⁰⁸ |
| 12F8 | 28.2 | 1.34E × 10⁰⁵ |
| 13G4 | 8.8 | NB |
| 18D7 | 7.4 | NB |
| 19E5 | 34.8 | NB |
| 29C7 | 20.5 | NB |

Abbreviations: NB, no binding.
Note:
This table lists the hybridoma, the corresponding total protein concentration in the supernatants and the affinity coefficient (KD (M)) as estimated by Octet equilibrium binding assays (see ELN EXP-20-BD1634).

### VIII. Example 8: Hybridoma Sensitivity and Dilution Series Screens

In order to evaluate the relative binding sensitivity of hybridoma culture supernatants to scFv-14, supernatants were serially diluted and screened against NTD T cells, scFv-14-containing CAR T cells, or FMC63-containing CAR T cells. Polyclonal sera from the mouse immunization (Abveris™ KIT19025-1) were used as a positive control. Pooled normal sera from unimmunized mice, conditioned media (CM) from an irrelevant hybridoma, and a mouse IgG isotype control were used as negative controls.

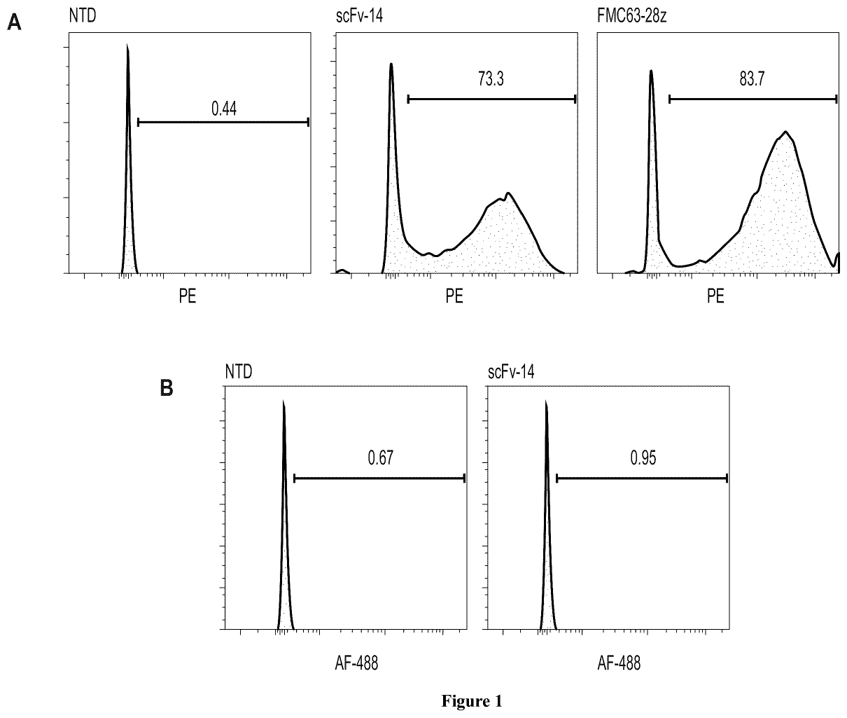

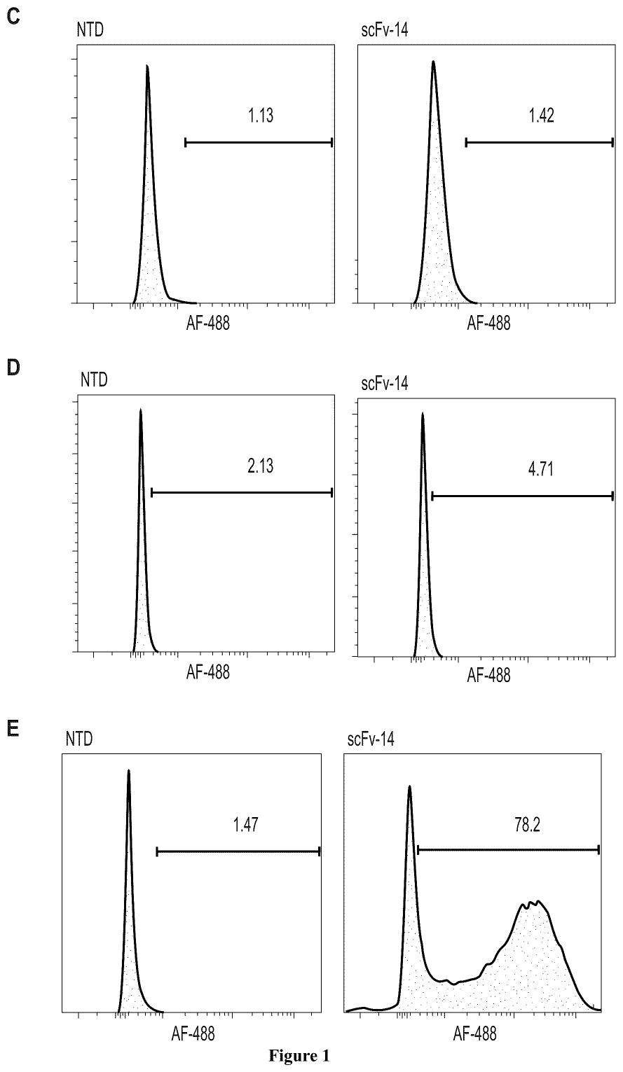

FIG. 1, panel A shows that both scFv14- and FMC63-bearing CARs are expressed by the transduced T cells (73.3% and 83.7% LC⁺, respectively) as determined by staining with the control anti-linker antibody LC. LC is a custom-made antibody that binds the peptide linker between the light and heavy chains of the CAR scFv, enabling assessment of total CAR transduction efficiency. The CM, isotype control, and normal mouse serum controls showed no binding to scFv-14 (FIG. 1, panels B-D). No binding of these controls to the FMC63-28z CAR cells was observed (data not shown). As expected, scFv14-bearing CAR T cells stained positively with the polyclonal antisera (FIG. 1, panel E). None of the supernatants showed binding to NTD T cells (data not shown) or T cells expressing the FMC63-bearing CAR (data not shown).

Since it is possible that relatively poor binding may be caused by low antibody concentration in the supernatants, a range-finding preliminary screen was performed to determine a suitable range to test the relative sensitivity in the serial dilution assays. Based on the results of the preliminary screen (data not shown), the 25 hybridoma culture supernatants were binned into 2 groups; 1 group of 9 of the 25 supernatants were tested in a 2-fold dilution series beginning at 1:500 (v/v) dilution with fluorescence-activated cell sorting (FACS) stain buffer. Binding to scFv-14 was determined by staining with goat anti-mouse IgG coupled to AF-488 (data not shown). The remaining 16 were tested in a 2-fold dilution series beginning at 1:125 (v/v) dilution with FACS stain buffer. Binding to scFv-14 was determined by staining with goat anti-mouse IgG coupled to AF-488 (data not shown).

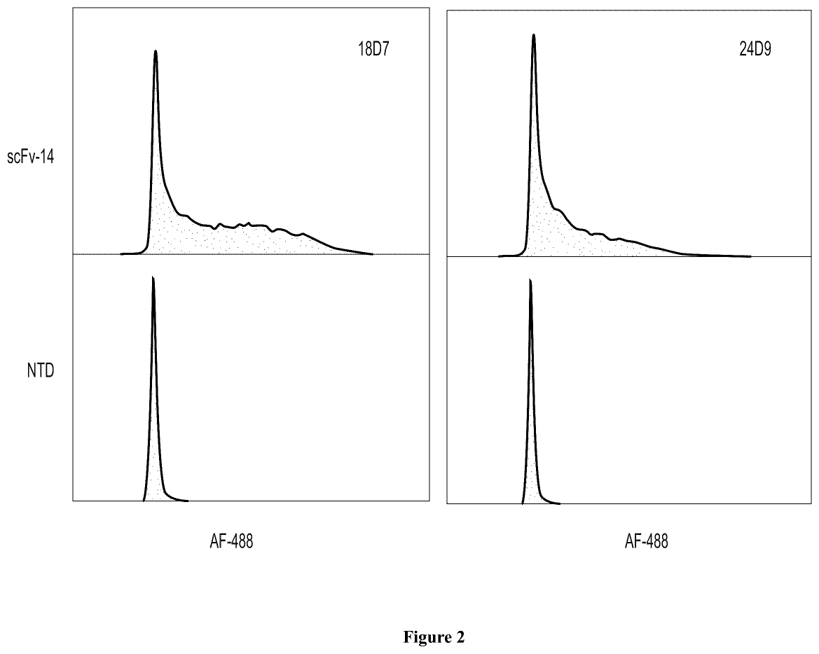

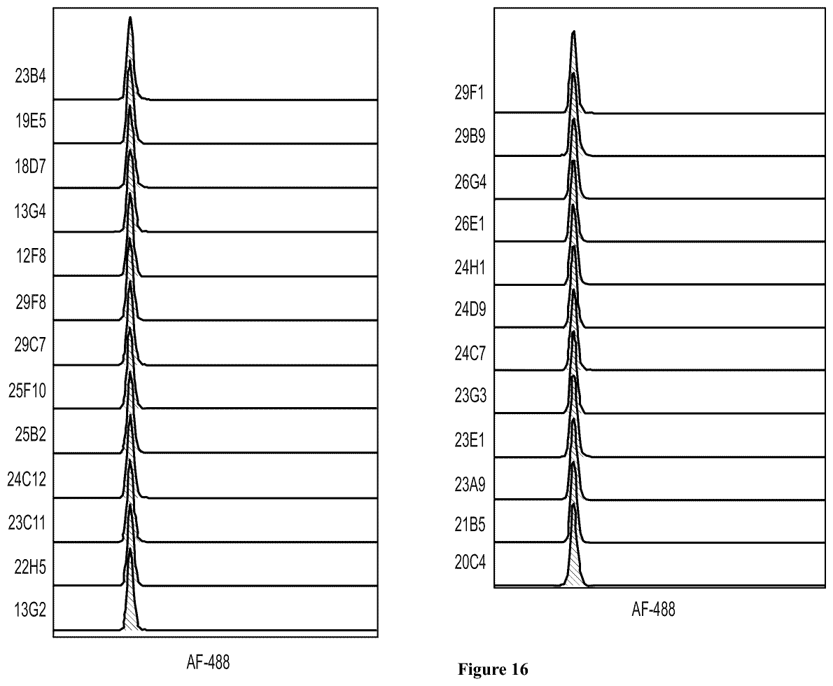

As a final check, the supernatant that showed no binding in the first dilutions series (29C7) as well as the 12 samples that had no binding in the second dilution series (23A9, 23G3, 24D9, 24H1, 26G4, 27B9, 12F8, 13G4, 18D7, 19E5, 20C4, and 21B5) were tested at a single dilution of 1:2 with stain buffer. This final dilution revealed 2 supernatants that showed binding to an scFv14-bearing CAR but not to NTD controls (see FIG. 2) or to a FMC63-bearing CAR (FIG. 16). Hybridomas that demonstrated binding to scFv14 at the lowest dilution in each of their respective series, but not to NTD T cells or FMC63-28z, were selected for the specificity screen.

The results from the dilution series show that overall, 14 hybridoma supernatants bind to scFv-14, but neither to the NTD controls or to the anti-CD19 CAR T cells. The following hybridomas were further chosen for the specificity screen: 13C2, 22H5, 23C11, 24C12, 25B2, 25F10, 29F8, 29F1, 23D4, 23E1, 24C7, 26E1, 18D7, and 24D9.

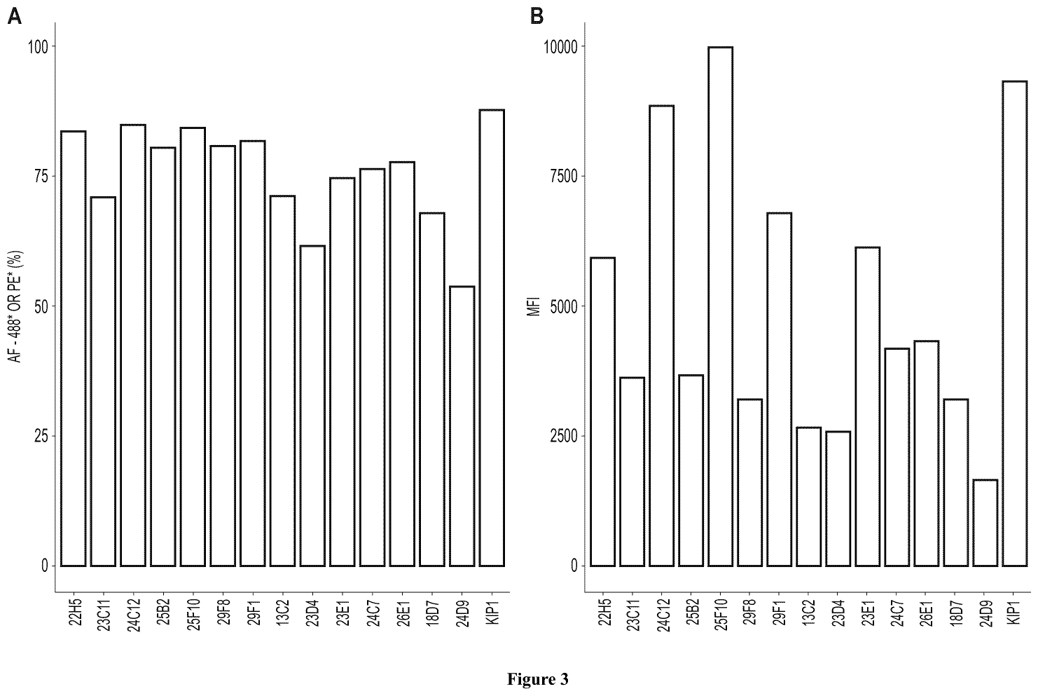

There were large shifts in the mean fluorescence intensity (MFI) between the hybridomas chosen. FIG. 3 summarizes binding at the highest concentration tested for each of the 14 hybridomas selected for specificity screening compared with the MFI of the samples. Shown are the fraction of cells that are AF-488+(see FIG. 3A) and the MFI for the AF-488⁺ populations in each sample (see FIG. 3B).

### IX. Example 9: Hybridoma Specificity Screen

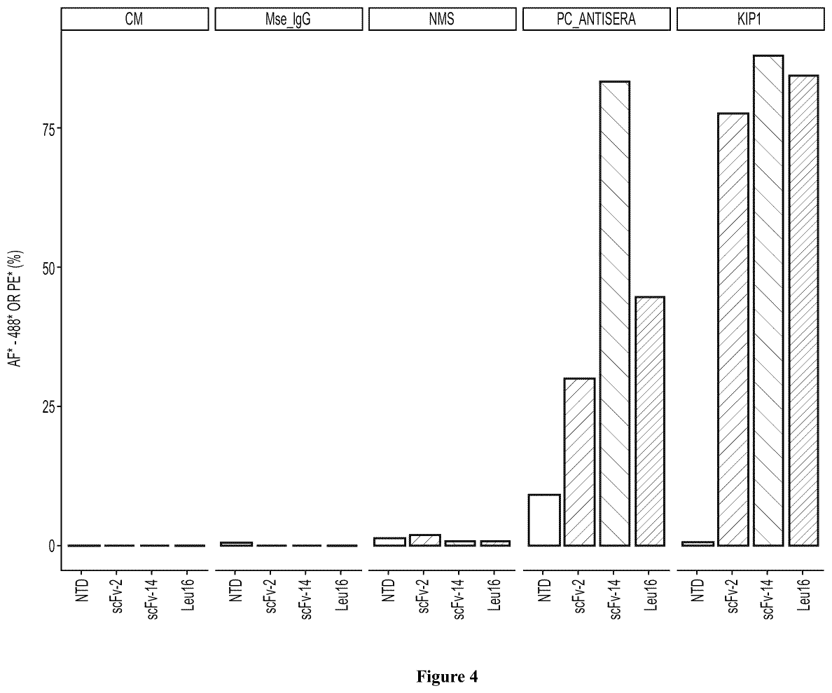

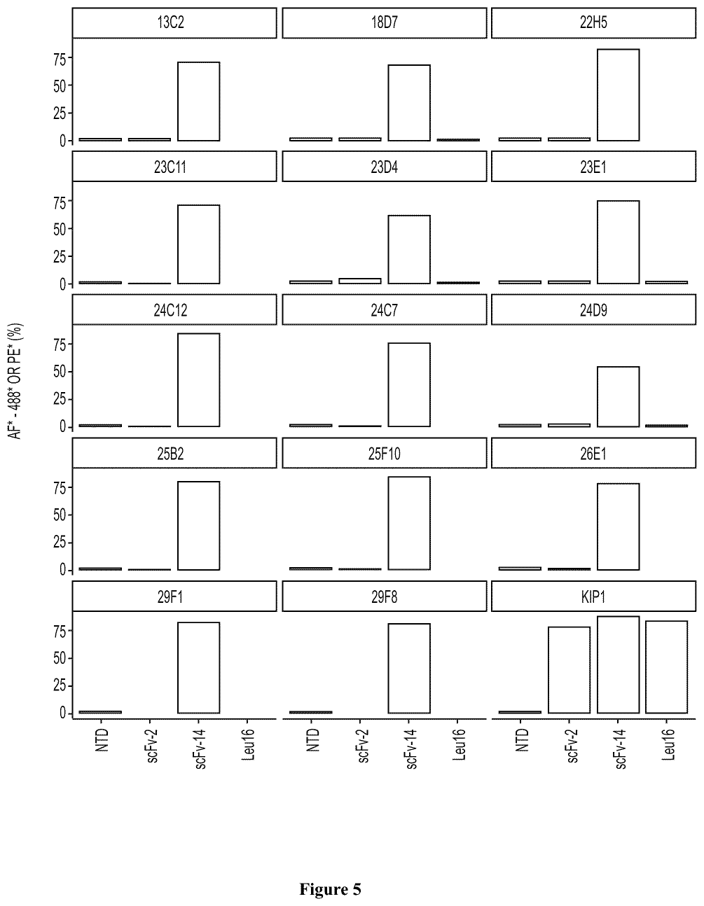

In order to evaluate the specificity to scFv14, supernatants were screened for binding to T cells expressing the scFv14-bearing CAR, the irrelevant anti-CD20 scFv2-bearing or Leu16-bearing CARs or NTD T cell controls. Pooled polyclonal post-immune antisera was used as a positive control while negative controls consisted of pooled pre-immune mouse sera, CM from an irrelevant hybridoma, and a mouse IgG isotype control (see FIG. 4). Overall CAR expression for the scFv2-, scFv14- and Leu16-bearing CAR T cells, as determined by LC PE staining, was 77.7%, 88.0%, and 84.5%, respectively (see FIG. 4). All 3 CARs showed some binding to polyclonal antisera. Since the immunogen was an anti-CD20 CAR, the polyclonal antiserum is expected to react to various shared domains in the CARs tested in this assay. Subsequently, the 14 hybridomas showing binding to scFv14 in the dilution series were screened at the highest concentrations used in the series as herein (see FIG. 5). LC PE⁺ control was included as a reference to determine the percentage expression of each CAR. The results indicate that all 14 hybridoma supernatants bind specifically to scFv-14, but not to any of the negative controls.

### X. Example 10: Selection of Hybridomas for Sequencing and Antibody Production

The goal of the dilution series and selection screening described in the examples herein was to choose candidate hybridomas for production. Based on these results, 10 of the 14 hybridomas tested for specificity were chosen for sequencing and antibody production. Some clones were not selected if the sequence was too similar to another clone or it appeared to be a weak binder in the hybridoma supernatant screening. The 10 hybridomas included 18D7, 22H5, 23C11, 23E1, 24C12, 24C7, 25B2, 25F10, 29F1, 29F8.

### XI. Example 11: Antibody Binding Confirmation and Selection for Characterization

In order to confirm selectivity of the antibody clones to scFv-14, they were screened against NTD T cells or T cells expressing CARs bearing either scFv-14, scFv2, or FMC63. CAR expression was determined by LC staining and was found to be 68.4%, 67.5%, and 67.7% respectively. In flow cytometry experiments, antibody clones 24C12, 29F1, 24C7, 23E1, 23C11, 18D7, 25B2, and 29F8 showed specific binding to scFv-14. Based on these results, all 8 clones were sent to BD Biosciences™ (San Diego, CA) for conjugation to PE and FITC.

### XI. Example 12: Post-Conjugation Characterization of 24C12

Of the 8 recombinant antibodies sent to BD™ for conjugation to fluorochromes, only 5 had arrived at Kite Pharma™ in time for inclusion in this report. Although all 5 of the anti-scFv-14 antibodies were found to be selective to the anti-CD20 scFv component of KITE-363 (data not shown) based on overall superior binding characteristics, 1 of the antibodies, 24C12, was chosen for final characterization.

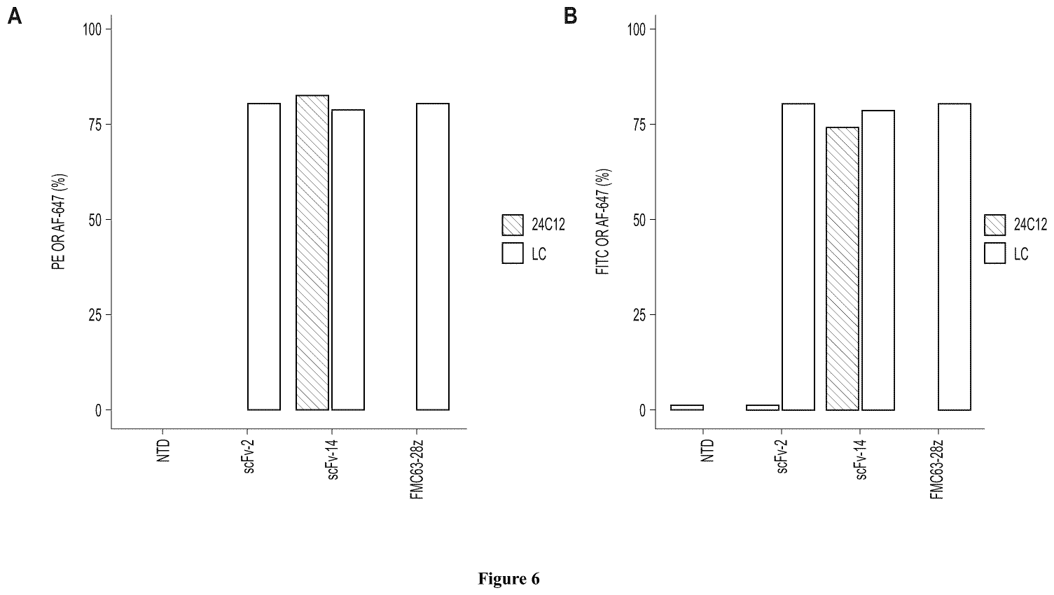

Antibody clone 24C12 conjugated to either PE or FITC was screened against healthy donor T cells that were left NTD or transduced to express CARs bearing either scFv2, scFv14, or FMC63. Antibody 24C12 shows specific binding to scFv14 regardless of fluorophore conjugate (see FIG. 6). The fraction of PE⁺ (see FIG. 6A) or FITC⁺ (see FIG. 6B) cells at the highest concentration of antibody tested (256 ng/mL) shows selective binding to an scFv14-bearing CAR and lack of binding to irrelevant CARs bearing scFv2 or FMC63. For reference, overall CAR expression in scFv2-, scFv14- and FMC63-bearing CAR transduced T cells, as determined by staining with LC-AF647, was 80.6%, 78.7%, and 80.7%, respectively (see FIG. 6).

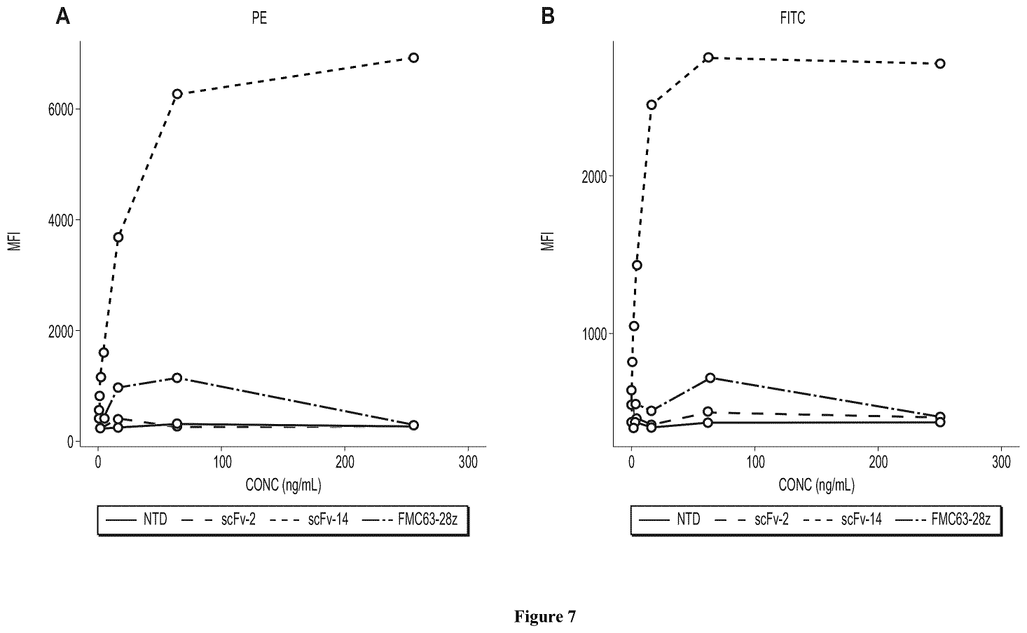

Serial dilutions of 24C12 PE (see FIG. 7A) and 24C12 FITC (see FIG. 7B) show that both are selective for scFv14.

### XIII. Example 13: Recombinant Protein Immunogen and Screening Reagent Generation

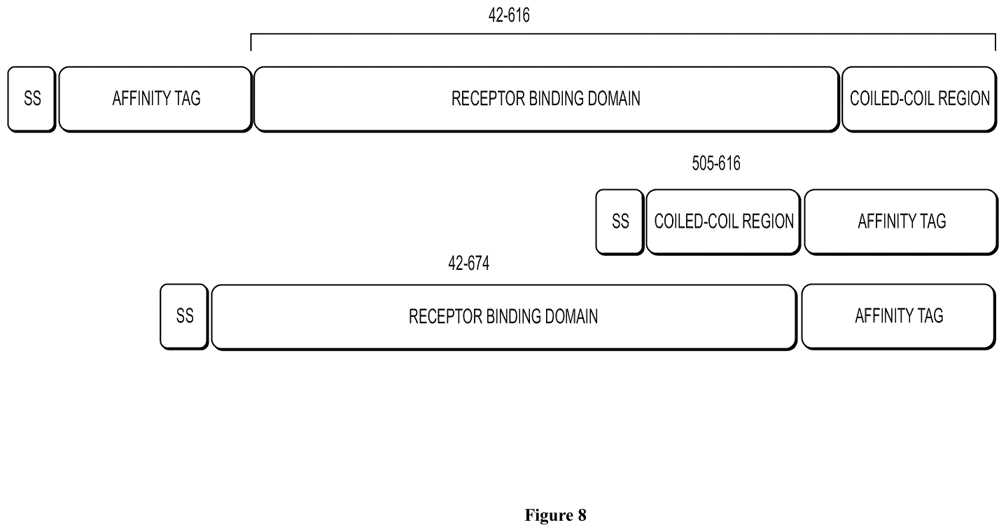

To facilitate the development and screening of antibodies that recognize the envelope protein, Gibbon ape leukemia virus (GALV) gp70 (Uniprot P21415), several recombinant proteins were designed, expressed and purified from human Expi293 cells containing murine Fc, human mono Fc or His tags, to be used as potential immunogens and screening reagents. These proteins included the receptor binding domain (RBD) of GALV gp70 (residues 42-474), the coiled-coil (CC) domain of GALV gp70 (residues 505-616) or the entire predicted viral surface exposed portion of GALV gp70 encompassing the RBD+CC domains (residues 42-616) (FIG. 8). The CC domain is composed of heptad repeats which associate to form a non-covalent trimer. Analytical size exclusion chromatography demonstrated that the CC domain containing recombinant proteins formed trimers.

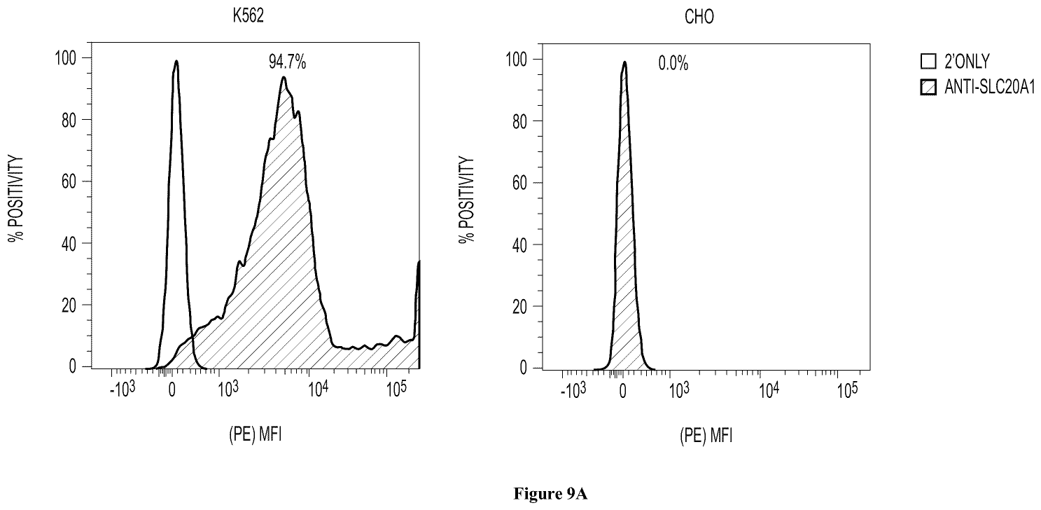

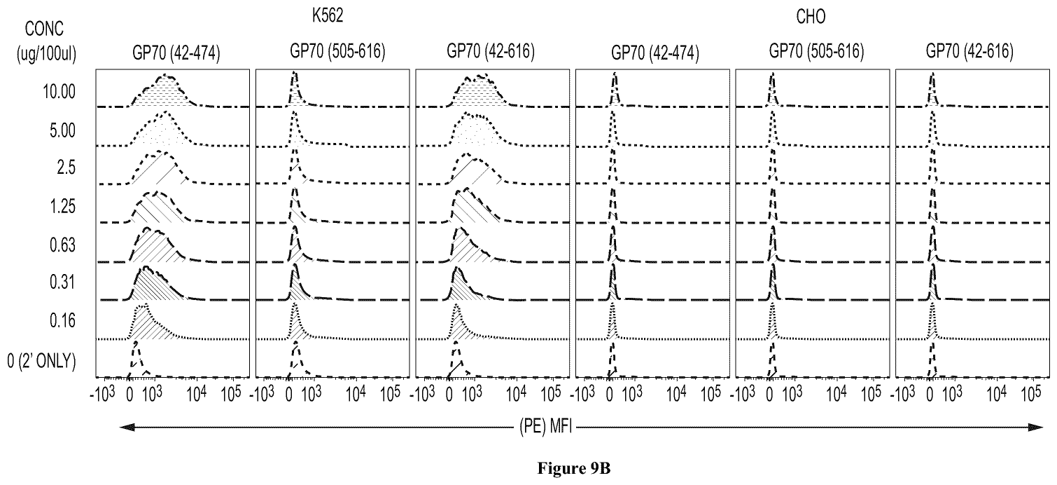

K562 cells expressing endogenous SLC20A1 (PIT1) receptor or a negative control cell line (CHO) were stained with a dilution series of each recombinant protein containing a human monoFc tag and fluorophore conjugated anti-human Fc secondary antibody. An anti-SLC20A1 antibody (Proteintech, cat #12423-1-AP) was included as a control. Recombinant GALV gp70 proteins containing the RBD bound to K562 cells in a dose dependent manner, while the protein consisting of only the CC domain did not. No binding was observed to the negative control cells (FIGS. 9A-9B). Taken together this data suggested proper structure and function, giving us confidence in our reagents for antibody generation and screening.

### XIV. Example 14: Hybridoma Campaign and Screening

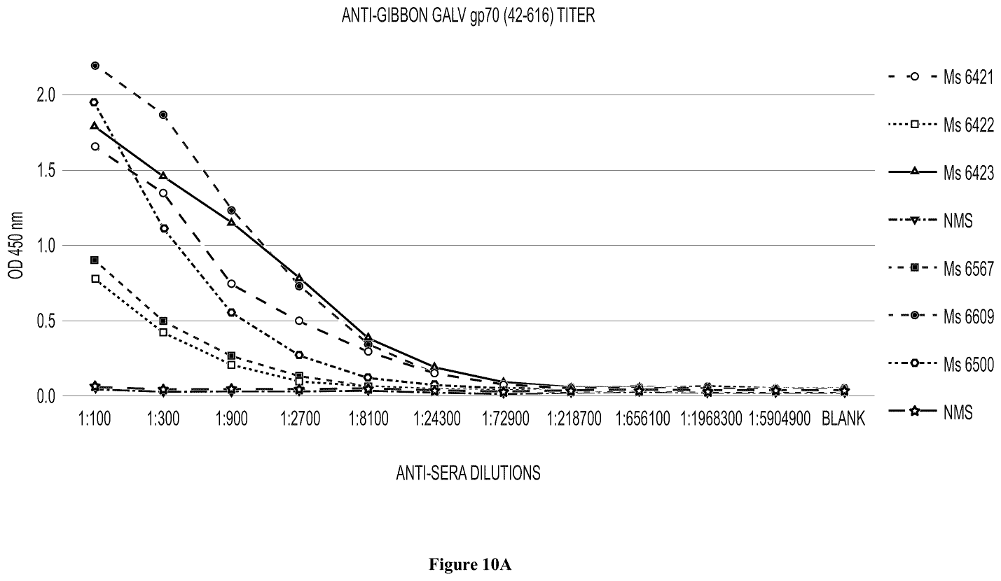

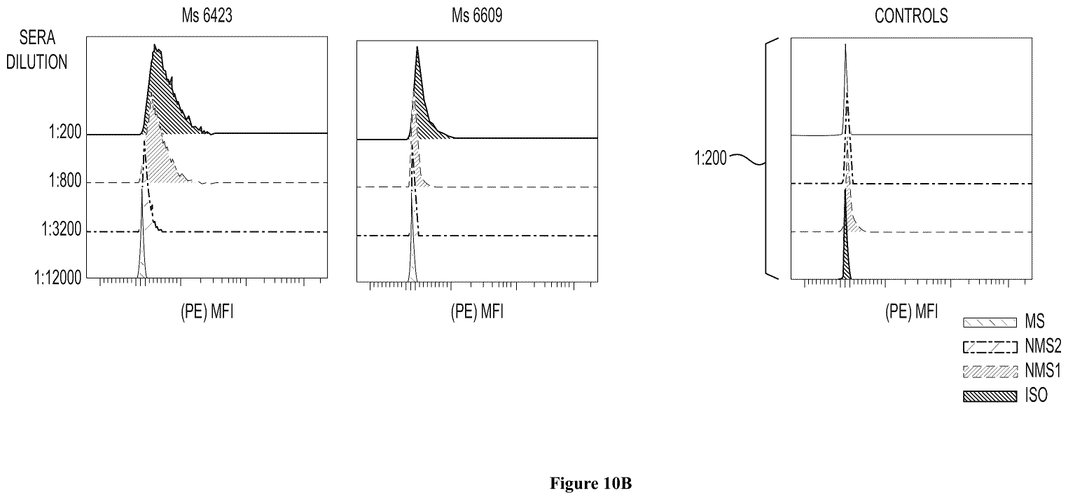

Abveris DiversimAb™ and DiverGimab™ hyperimmune mice (Canton, MA) were immunized with replication incompetent, empty retroviral (RVV) particles (no payload) containing the envelope protein, GALV gp70. Mice were boosted with recombinant murine IgG2a Fc tagged GALV gp70 protein (residues 42-616) or the empty RVV particles. Mouse serum was titer tested by dilution series by indirect ELISA coated with recombinant soluble huIgG1 monoFc tagged Gibbon GALV gp70 proteins (residues 42-616, residues 42-474 or residues 505-616). An irrelevant huIgG1 monoFc tagged protein was used as a negative control. Mouse serum was also tested by flow cytometry in a dilution series for sensitivity to a PG13-based stable packaging cell line, constitutively producing viral particles containing the GALV gp70 envelope protein. PG13 cells were stained with normal mouse serum (NMS), no staining (NS), or an isotype control as negative controls. Two mice were chosen for hybridoma fusion based on positive ELISA and flow cytometry data (FIG. 10A-10B).

### XV. Example 15: Hybridoma Supernatant Screening

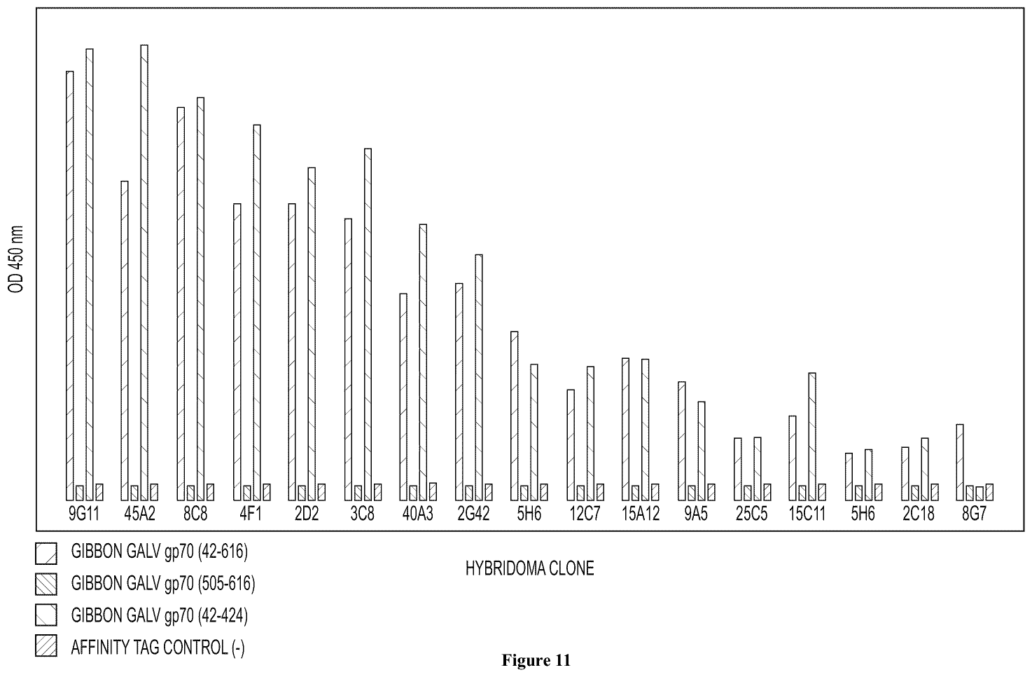

Thousands of hybridoma fusion supernatants were screened using indirect ELISA coated with recombinant soluble huIgG1 monoFc tagged Gibbon GALV gp70 proteins (residues 42-616). An irrelevant huIgG1 monoFc tagged protein was used as a negative control. Only 60 positive binders were identified, which were then subjected to a secondary ELISA screen coated with recombinant soluble huIgG1 monoFc tagged Gibbon GALV gp70 proteins (residues 42-616, residues 505-616 or residues 42-474). An irrelevant huIgG1 monoFc tagged protein was used as a negative control. This further narrowed the list of positive antibodies to 17 clones (FIG. 11). Following this analysis, one of the hybridoma cell lines was found to be non-viable, decreasing the number of positive hybridomas to 16.

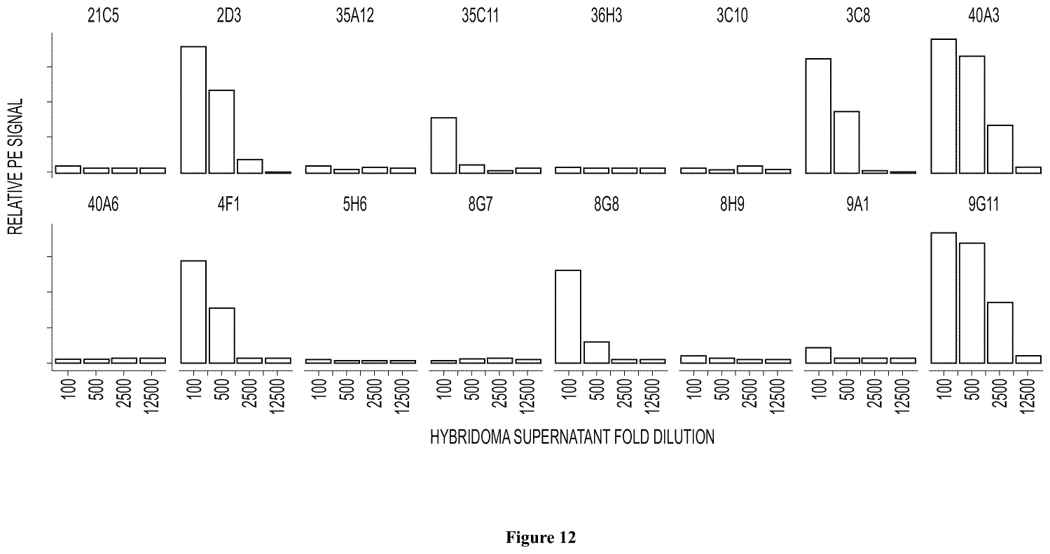

Supernatants of the 16 viable positive hybridoma lines tested in the ELISA-based screen were screened by flow cytometry in a dilution series for sensitivity to the PG13-based stable packaging cell line, constitutively producing viral particles containing the GALV gp70 envelope protein (FIG. 12). NIH-3T3 parental cells were used as a negative control and incubated with the same hybridoma supernatants and serially diluted with stain buffer. Negative controls consisted of an immunoglobulin (Ig) isotype control and normal mouse sera from two different mice (non-immunized). Cells were incubated with the diluted supernatants for 45 minutes at room temperature (RT), then harvested and washed twice with stain buffer. Supernatant and control samples were stained with PE conjugated secondary antibody. Data was analyzed using FlowJo™ software (BD, version 10.6) and events were systematically gated on cells (using forward scatter [FSC]-area by side scatter [SSC]-area plot), single cells (using FSC-area by FSC-height plot) and phycoerythrin (PE), where the gating threshold was set based on the negative control cells. 8 out of 16 of the viable hybridoma supernatants that were positive by ELISA were also confirmed positive by flow cytometry. The positive clones in this experiment were: 35C11, 40A3, 8G8, 9A1, 9G11, 4F1, 2D3 and 40A6.

### XVI. Example 16: Recombinant Antibody Sensitivity and Specificity Testing

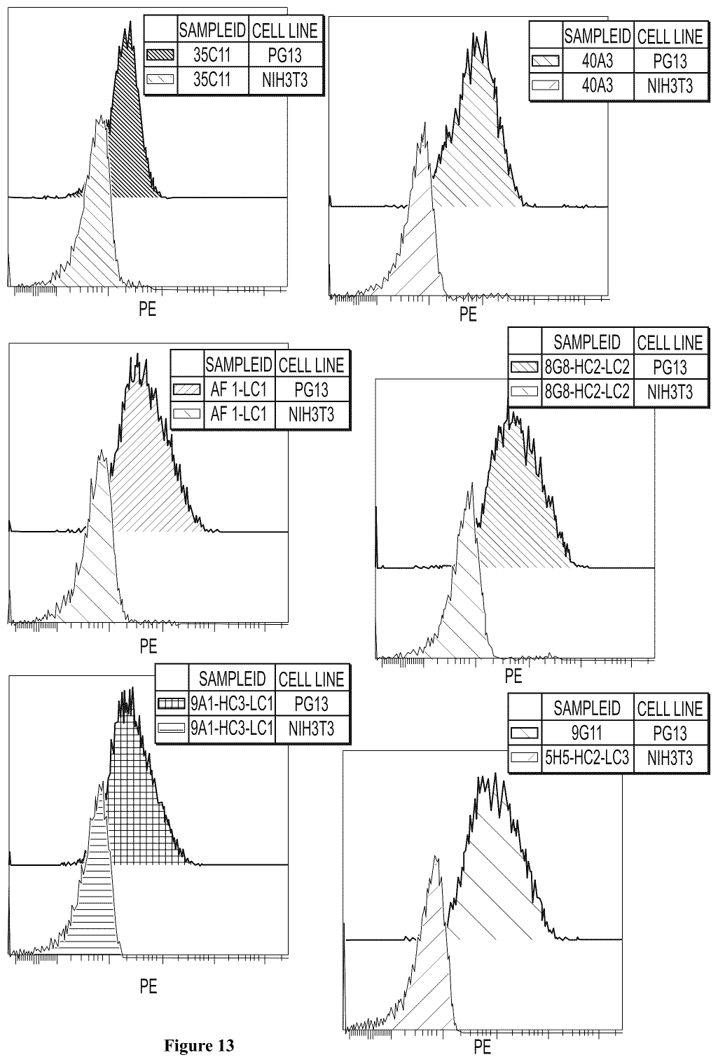

All hybridoma clones positive for binding GALV gp70 by ELISA were sequenced using next generation sequencing (NGS). After sequencing of the antibody variable heavy chain (VH) and variable light chain (VL) domains from the hybridomas, antibodies were cloned into standard mammalian expression vectors as murine IgG2a format and manufactured at small scale. All unique VH and VL sequences resulted in a panel of 12 recombinant clonal antibodies produced, which were functionally tested at small scale, if purified yields were high enough for testing. To test the sensitivity and specificity of the purified antibodies, a single 10-fold dilution of each antibody was incubated with PG13 cells or NIH-3T3 cells as a negative control (FIG. 13). The following clones had sufficient purified yields for testing and were positive for PG13 staining: 8G8, 9A1, 40A3, 4F1, 35C11 and 9G11.

### XVII. Example 17: Purified Antibody Sensitivity and Dilution Series Screen

To confirm the small-scale manufacturing and screening results, and to retest the clones with poor small scale purified yields, the antibodies were manufactured at larger scale. Briefly, antibodies were expressed in ExpiCHO cells using the ExpiFectamine CHO Transfection Kit (Thermo Fisher, Cat. No A29133). Antibodies were affinity purified over a HiTrap MabSelect SuRe column (Cytiva, Product No 11003493) followed by size exclusion chromatography on a HiLoad Superdex 200 16/600 (Cytiva, Product No. 28989335). A panel of 9 antibodies was successfully produced and the purity of each antibody was determined to be >95% by SDS-PAGE gel and analytical SEC (SEC-UPLC). Antibodies were sterile filtered with 0.22 m filters and stored at −80° C. The following clones were selected for further analysis: 35C11, 3C8, 40A3, 40A6, 8G8, 9A1, 9G11, 2D3 and 4F1.

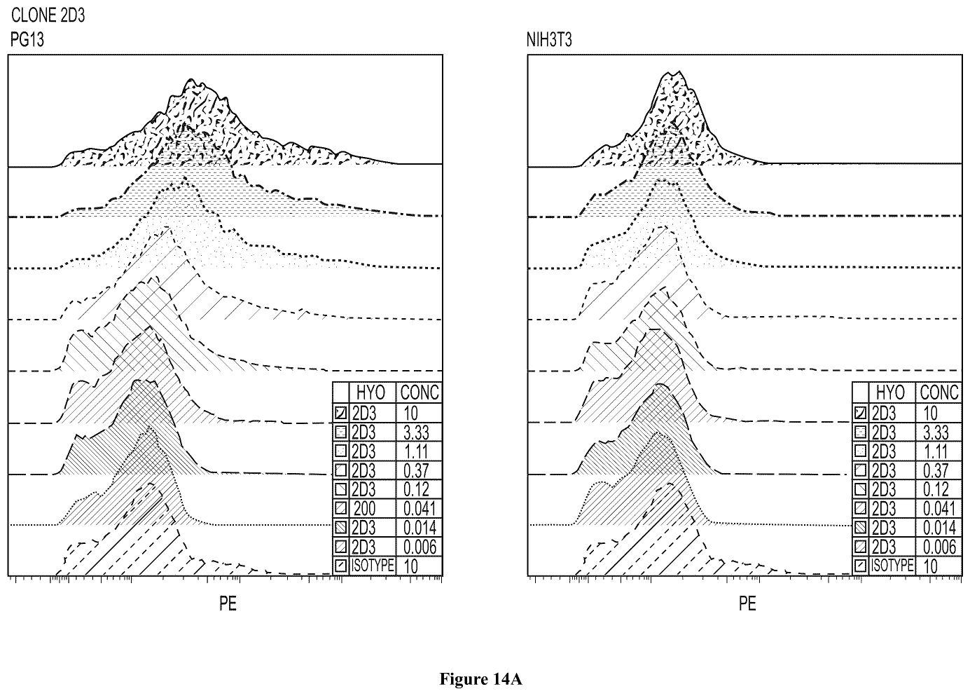

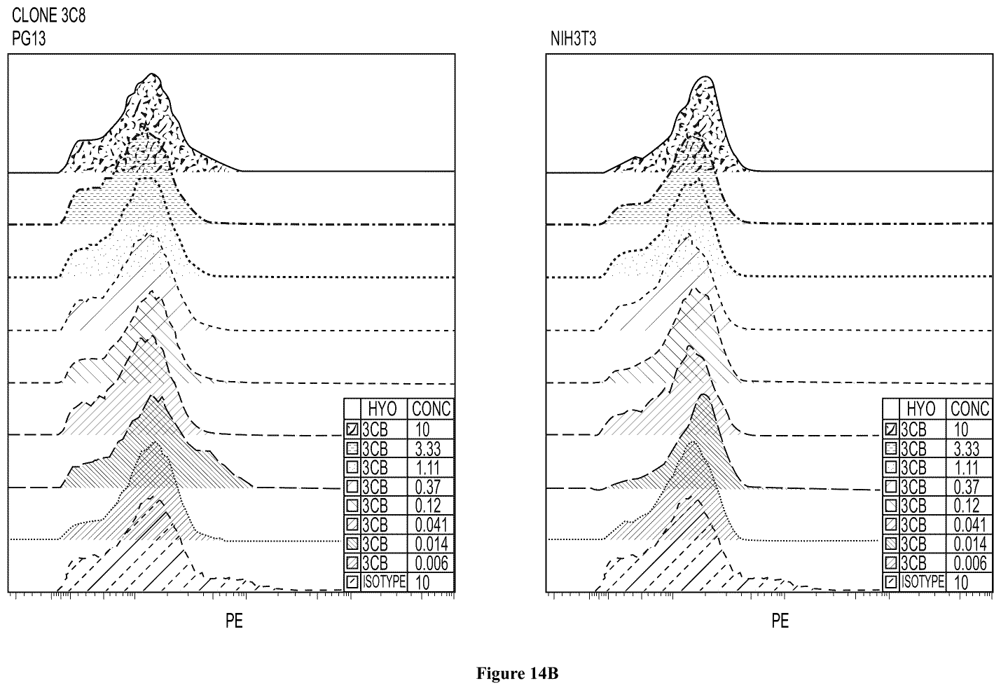

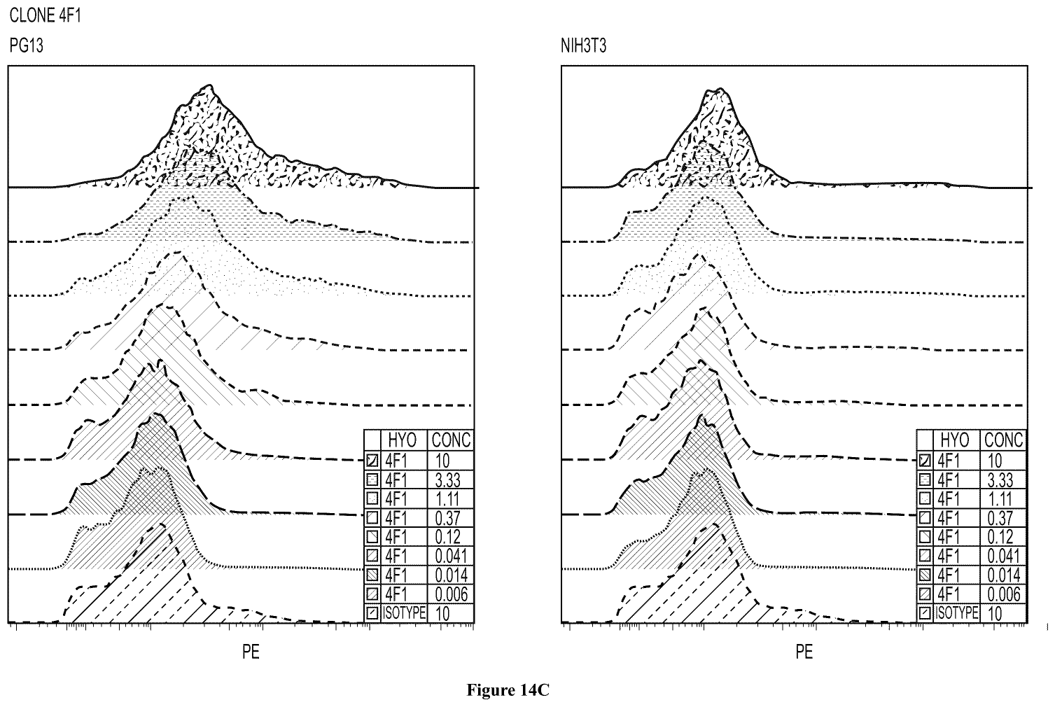

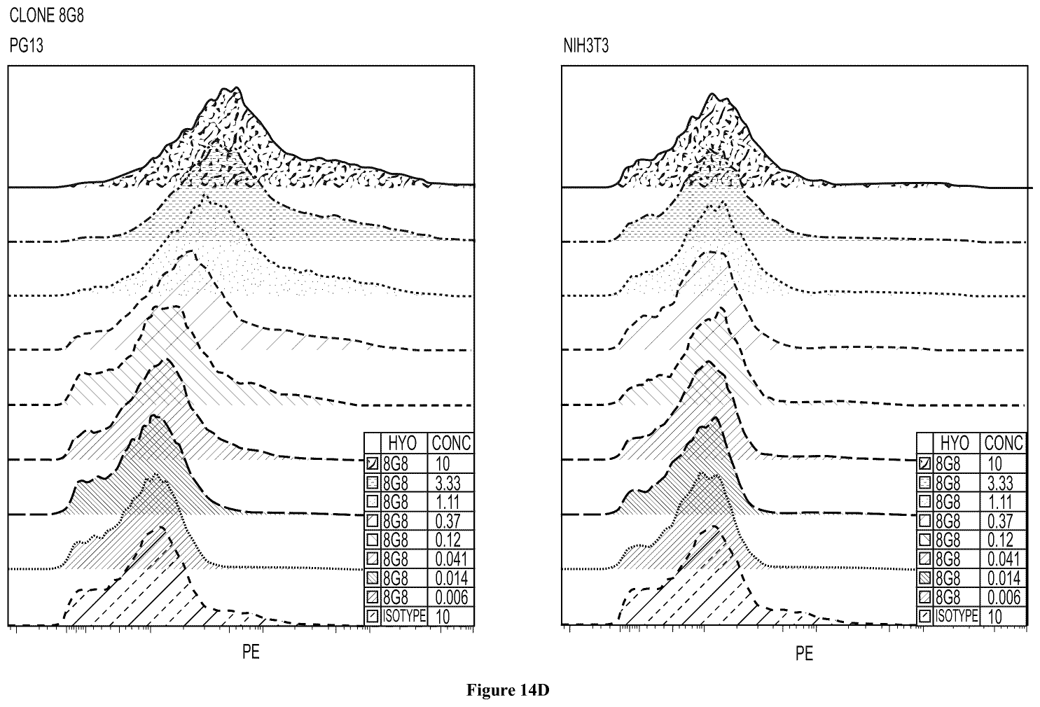

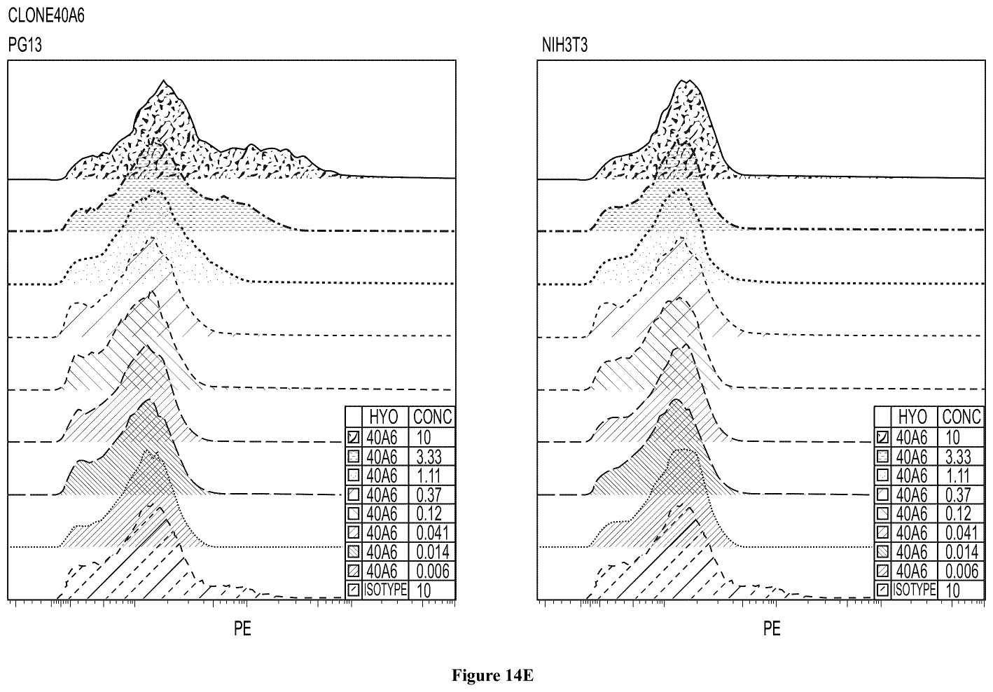

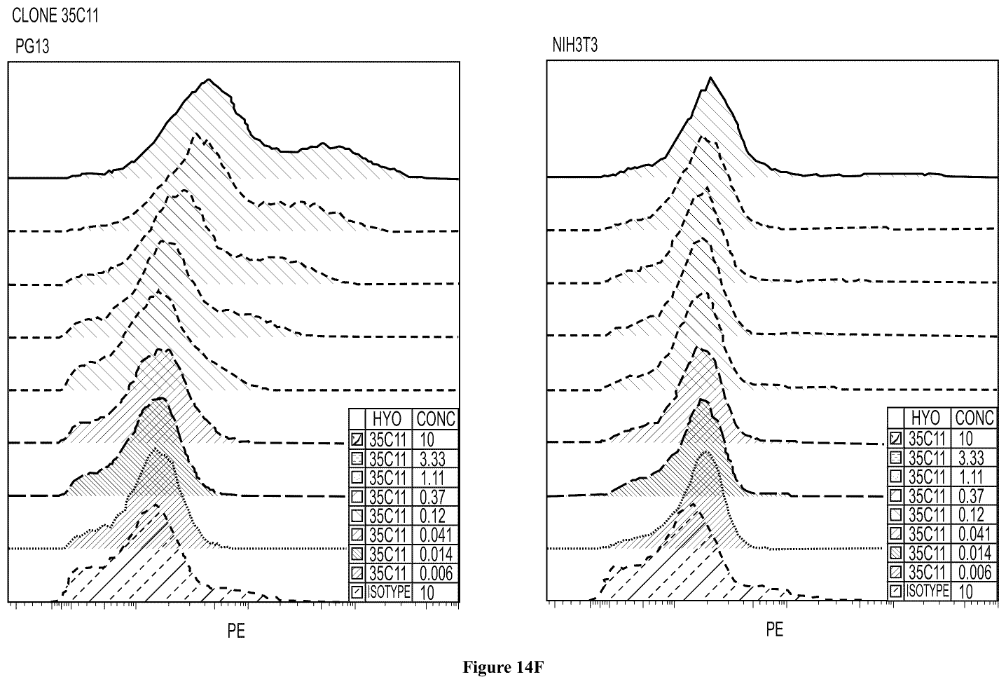

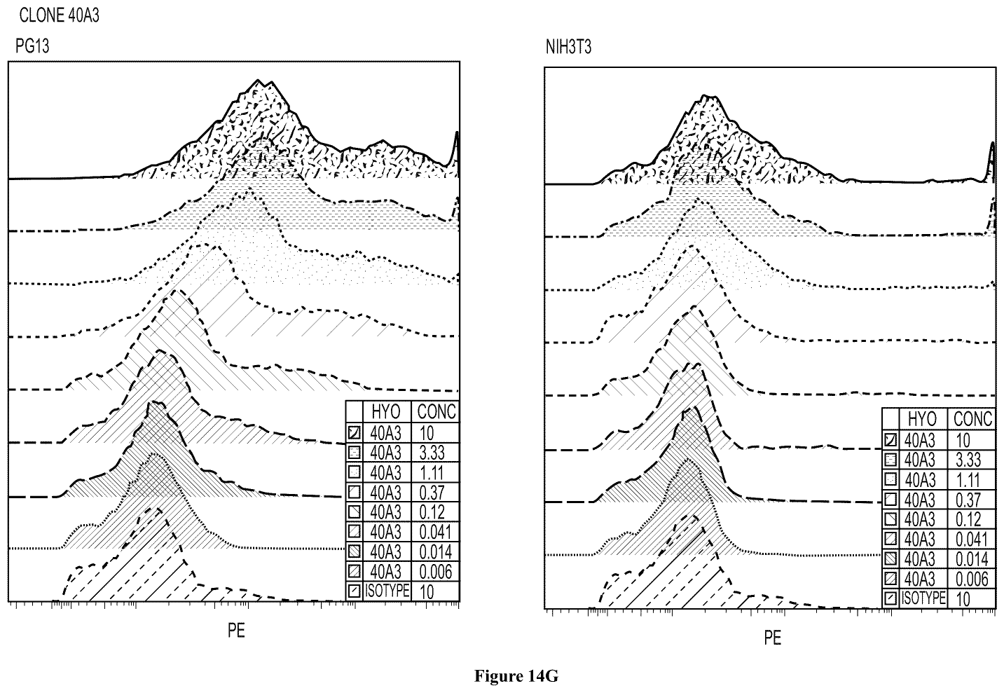

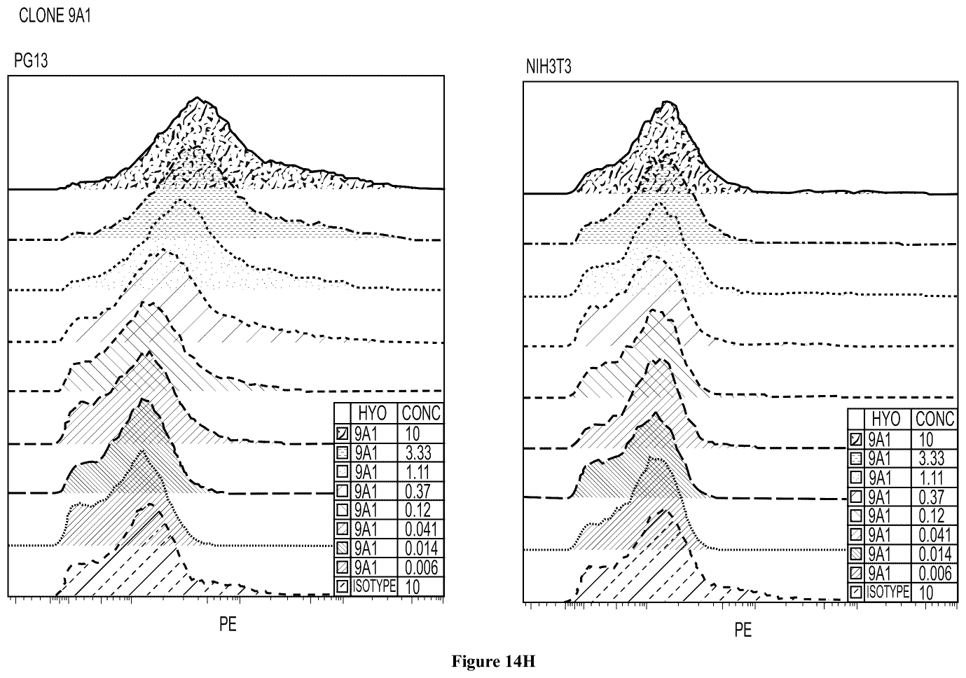

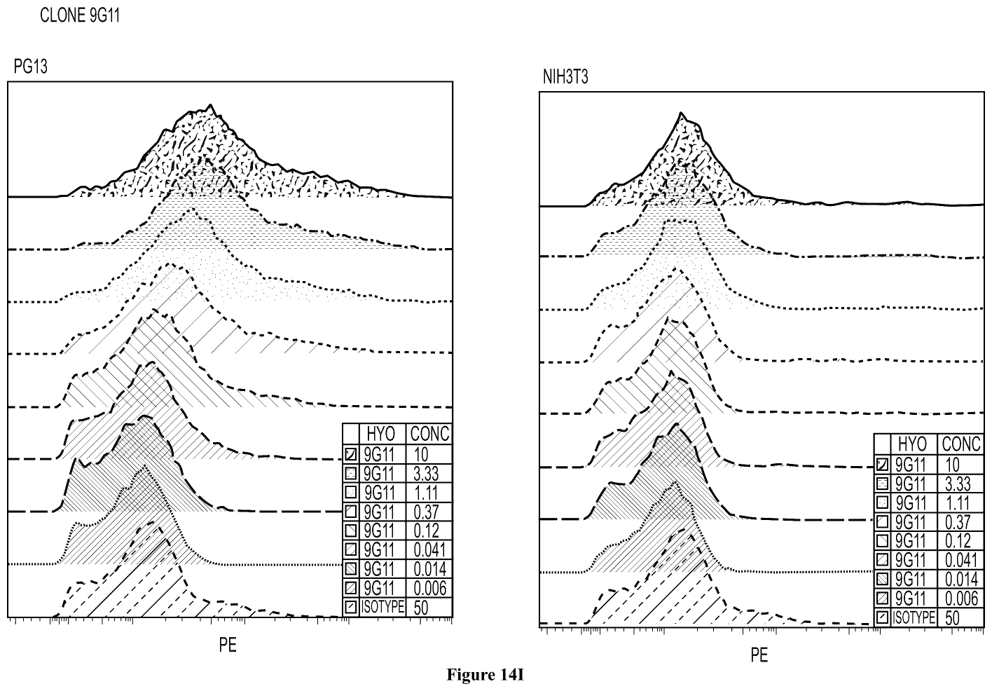

To test the sensitivity and specificity of the purified antibodies, a titration/3-fold dilution series was performed (top concentration of 10 ug/mL down to 0.005 ug/mL) and PG13 cells were incubated with each of the antibodies, or a mouse IgG isotype control at the top concentration (FIG. 14A-14I). NIH-3T3 cells were used as a negative control and incubated with the same purified antibody dilution series with stain buffer. Both cell lines were also stained with secondary antibody only (in the absence of primary antibodies) as a negative control. Cells were stained and analyzed.

### XVIII. Example 18: Purified Antibody Affinity Ranking and Specificity Testing Using BioLayer Interferometry

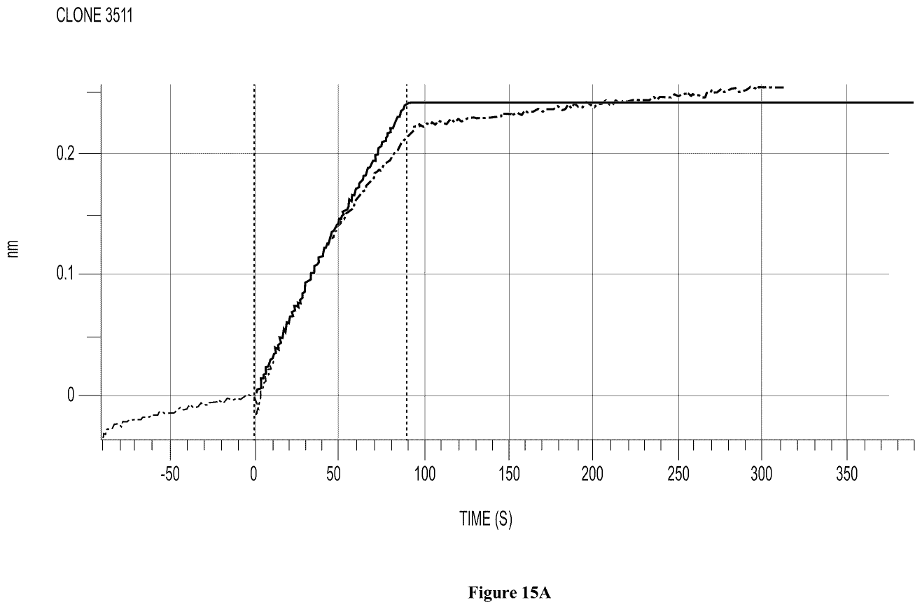

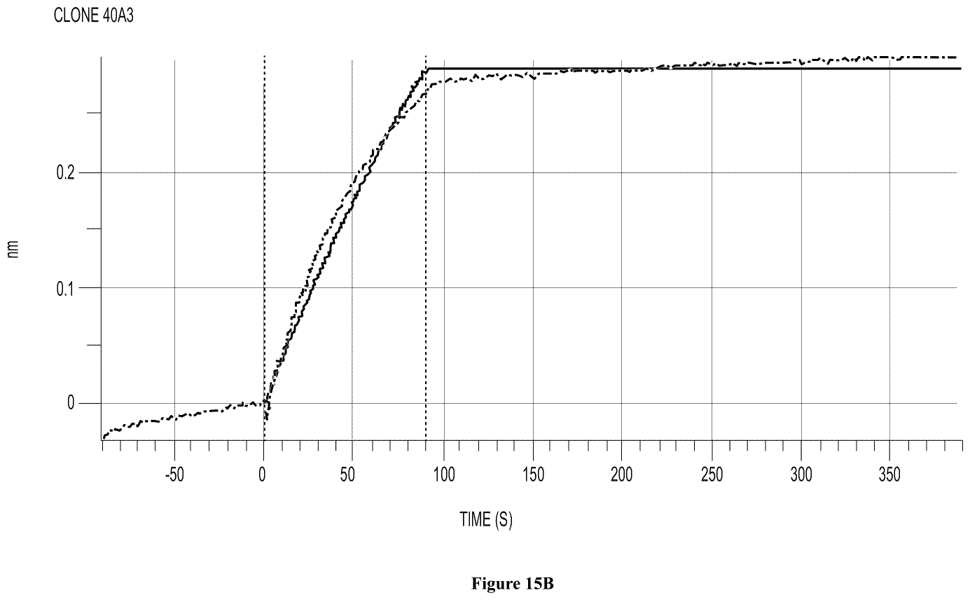

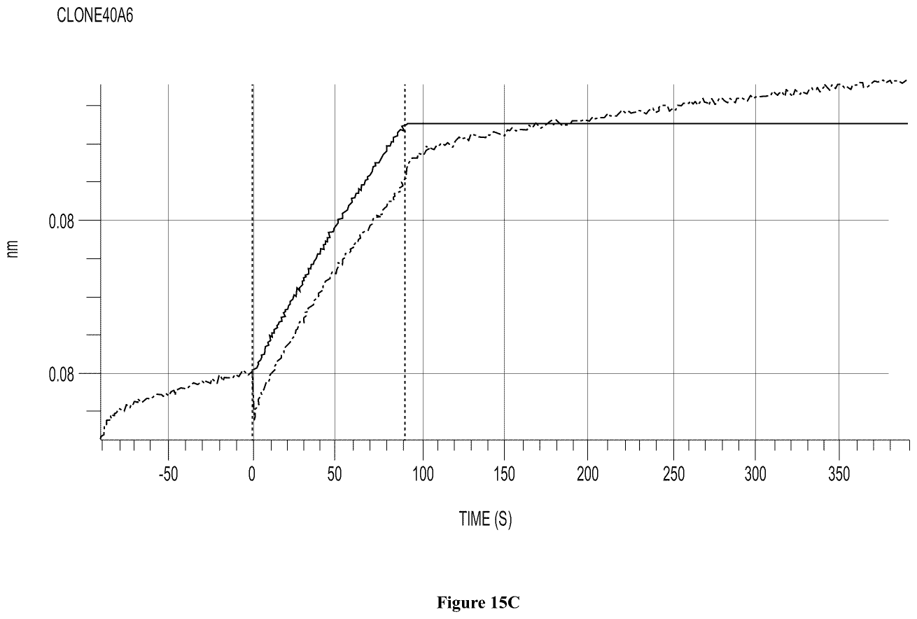

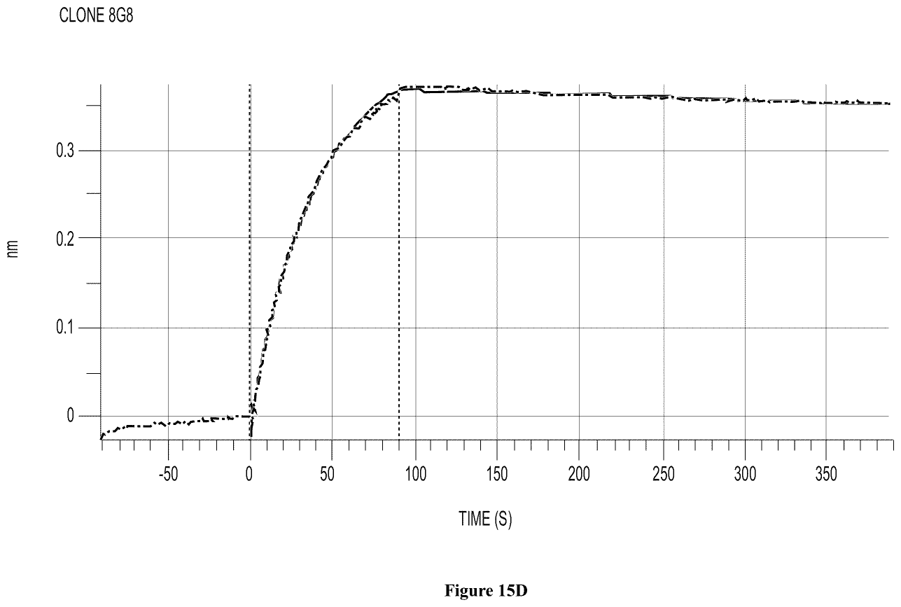

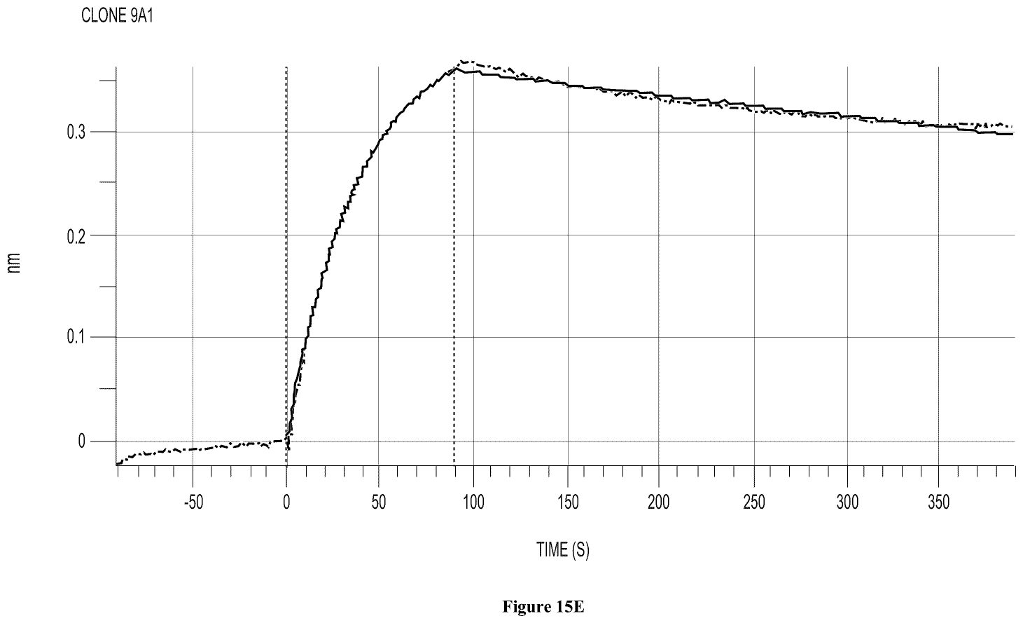

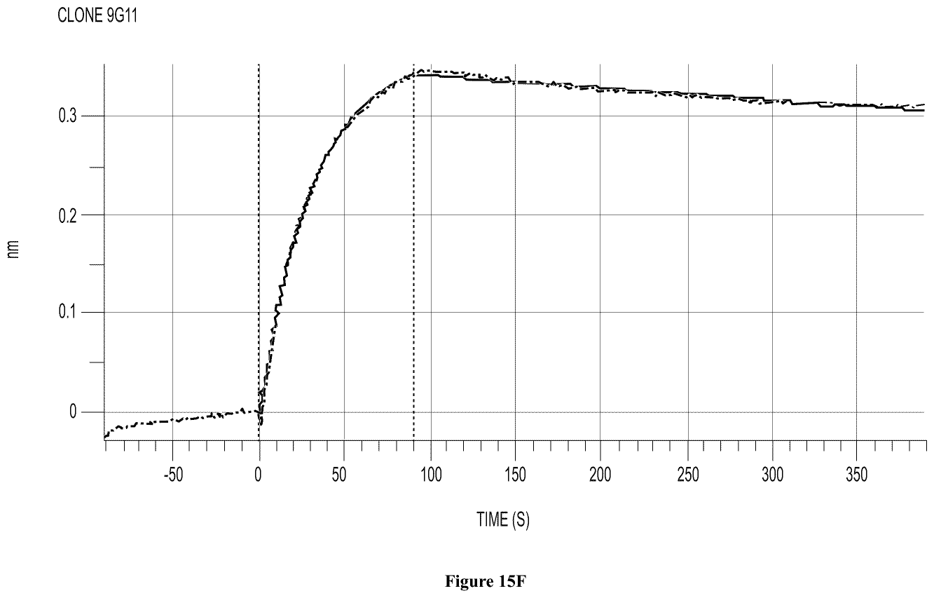

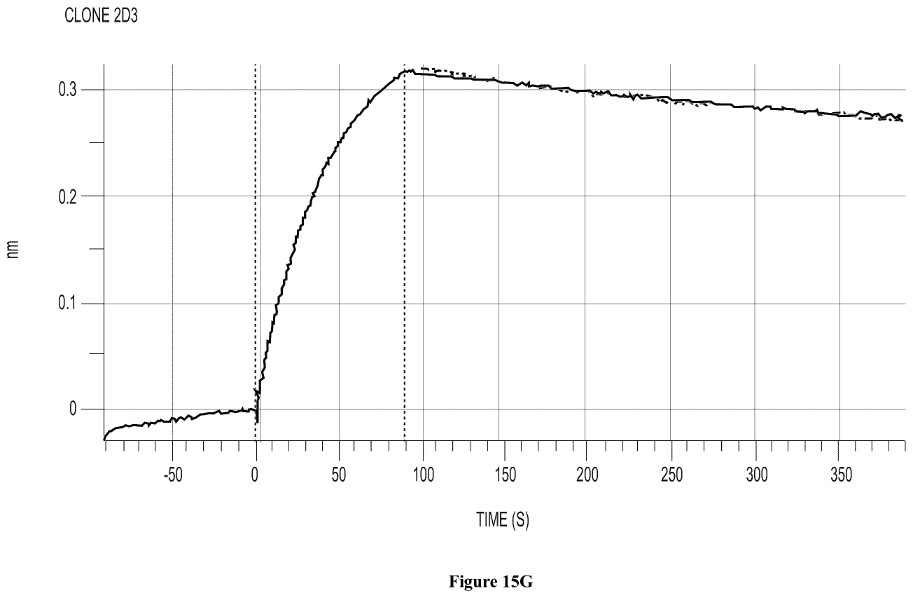

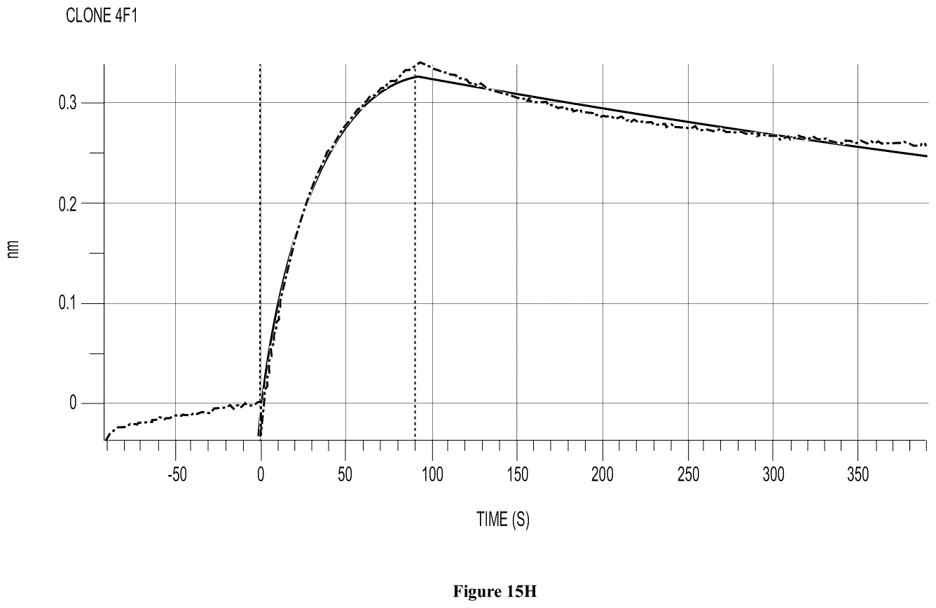

Relative affinity ranking, epitope binning and specificity of the purified antibodies were measured by BioLayer interferometry using an Octet Red96 (Sartorius). Briefly, the purified antibodies were loaded onto AMC biosensors (product number 18-5088) at 2 ug/mL and tested for binding to 100 nM recombinant soluble huIgG1 monoFc tagged Gibbon GALV gp70 protein analytes (residues 42-616, residues 42-474 or residues 505-616) (FIG. 15A-15H). A negative control analyte, soluble VSV-G (residues 17-467) huIgG1 monoFc tagged protein was used to confirm antibody specificity. Competition assays were performed to identify clones with unique or non-overlapping epitopes. These experiments identified clones 35C11, 8G8 and 40A3 to have non-overlapping or non-competing epitopes within residues 42-474 of GALV-gp70.

### XIX. Example 19: Purified Antibody Characteristics

A panel of 9 recombinant antibodies were successfully produced. The antibodies were characterized and screened according to the methods described in Example 18. Affinity values in Table 16 are for the antibody clone to hulgG1 monoFc tagged Gibbon GALV gp70 protein (residues 42-474).

TABLE 16
Purified Antibody Characteristics

| | Purified | | | |
|---|---|---|---|---|
| Antibody | Yield | | | |
| clone | (mg/L) | Kon (M−1 s−1) | Koff (s−1) | KD (M) |
| 35C11 | 7.8 | 5.15E+04 | 6.29E−07 | 1.22E−11 |
| 40A3 | 4.8 | 1.14E+05 | 6.00E−07 | 5.24E−12 |
| 40A6 | 5.5 | 3.83E+04 | 6.72E−07 | 1.75E−11 |
| 8G8 | 10.9 | 3.28E+05 | 6.31E−05 | 1.93E−10 |
| 9A1 | 37.5 | 3.18E+05 | 4.47E−04 | 1.40E−09 |
| 9G11 | 6.0 | 3.66E+05 | 2.57E−04 | 7.04E−10 |
| 2D3 | 42.0 | 2.81E+05 | 4.80E−04 | 1.71E−09 |
| 4F1 | 82.0 | 3.59E+05 | 6.07E−04 | 1.69E−09 |
| 3C8 | 37.1 | NB | NB | NB |

Abbreviations: NB, no binding.
Note:
This table lists the antibody clones and purified yields in mg/L when produced in ExpiCHO cells and two-step purified. Affinity values ((KD (M)) were estimated by Octet binding assays.

### XX. Example 20: Fluorophore Conjugation to Recombinant Antibodies

Three anti-GALV gp70 antibodies, clones 8G8, 40A3 and 35C11 were chosen as lead antibody candidates due to their binding properties conjugated to Dylight™ 650 (Thermno Fisher) following standard protocols to couple the fluorophore to primary amines on the antibody. Nonconjugated fluorophore was removed using size exclusion chromatography methods. Purity of each conjugated antibody was determined to be >95% analytical SEC (SEC-UPLC) and the degree of labeling was determined for each using spectrophotometric methods.

### XXI. Example 21: Fluorophore-Conjugated Antibody Characterization

For characterization of the fluorophore conjugated 8G8, 40A3 and 35C11 antibodies, PG13 cells were incubated with 200 ng of each fluorophore labeled antibody. NIH-3T3 cells were used as a negative control. Both cell lines were also stained with secondary antibody only (in the absence of primary antibodies) as a negative control. Cells were stained and analyzed (FIGS. 15A-15H).

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The foregoing written specification is considered to be sufficient to enable one skilled in the art to practice the invention. The foregoing description and Examples that follow detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. An isolated antigen binding molecule that binds to an anti-CD20 binding region, comprising:
a heavy chain variable (VH) sequence that has at least about 80% sequence identity to a sequence selected from the group consisting of SEQ ID NOs: 1-10; and
a light chain variable (VL) sequence that has at least about 80% sequence identity to a sequence selected from the group consisting of SEQ ID NOs: 11-20.

2. The isolated antigen binding molecule of claim 1, comprising a VH amino acid sequence having at least about 85%, at least about 90%, at least about 95%, at least about 96%, at least about 97%, at least about 98%, or at least about 99% identity to a sequence selected from the group consisting of SEQ ID NOs: 1-10.

3. The isolated antigen binding molecule of claim 1, comprising a VL amino acid sequence having at least about 85%, at least about 90%, at least about 95%, at least about 96%, at least about 97%, at least about 98%, or at least about 99% identity to a sequence selected from the group consisting of SEQ ID NOs: 11-20.

4. The isolated antigen binding molecule of claim 1, wherein the isolated antigen binding molecule comprises a heavy chain CDR1 selected from the group consisting of SEQ ID NOs: 21-41.

5. The isolated antigen binding molecule of claim 1, wherein the isolated antigen binding molecule comprises a heavy chain CDR2 selected from the group consisting of SEQ ID NOs: 42-65.

6. The isolated antigen binding molecule of claim 1, wherein the isolated antigen binding molecule comprises a heavy chain CD3 selected from the group consisting of SEQ ID NOs: 66-85.

7. The isolated antigen binding molecule of claim 1, wherein the antigen binding molecule comprises a light chain CDR1 selected from the group consisting of SEQ ID NOs: 86-99.

8. The isolated antigen binding molecule of claim 1, wherein the antigen binding molecule comprises a light chain CDR2 selected from the group consisting of SEQ ID NOs: 100-106, YAS, YTS, AAS, WAS, and NAK.

9. The isolated antigen binding molecule of claim 1, wherein the antigen binding molecule comprises a light chain CDR3 selected from the group consisting of SEQ ID NOs: 112-120.

10. The isolated antigen binding molecule of claim 1, further comprising a linker connecting the VH to the VL, wherein the linker comprises an amino acid sequence.

11. The isolated antigen binding molecule of claim 10, wherein the amino acid sequence of the linker comprises a sequence having at least about 80% sequence identity to SEQ ID NO: 121.

12. The isolate antigen binding molecule of claim 10, wherein the amino acid sequence of the linker comprises a sequence having at least about 80% sequence identity to SEQ ID NO: 126.

13. The isolated antigen binding molecule of claim 11, wherein the amino acid sequence of the linker comprises a sequence having at least about 85%, at least about 90%, at least about 95%, at least about 96%, at least about 97%, at least about 98%, or at least about 99% sequence identity to SEQ ID NO: 121.

14. The isolated antigen binding molecule of claim 12, wherein the amino acid sequence of the linker comprises a sequence having at least about 85%, at least about 90%, at least about 95%, at least about 96%, at least about 97%, at least about 98%, or at least about 99% sequence identity to SEQ ID NO: 126.

15. The isolated antigen binding molecule of claim 1, further comprising a detectable label selected from the group consisting of a fluorescent label, a photochromic compound, a proteinaceous fluorescent label, a magnetic label, a radiolabel, and a hapten.

16. The isolated antigen binding molecule of claim 1, wherein the isolated antigen binding molecule comprises a heavy chain CDR1 sequence selected from the group consisting of SEQ ID NOs: 25, 32, and 39.

17. The isolated antigen binding molecule of claim 1, wherein the isolated antigen binding molecule comprises a heavy chain CDR2 sequence selected from the group consisting of SEQ ID NOs: 46, 54, and 62.

18. The isolated antigen binding molecule of claim 1, wherein the isolated antigen binding molecule comprises a heavy chain CDR3 sequence selected from the group consisting of SEQ ID NOs: 70 and 80.

19. The isolated antigen binding molecule of claim 1, wherein the isolated antigen binding molecule comprises a light chain CDR1 sequence selected from the group consisting of SEQ ID NOs: 90 and 98.

20. The isolated antigen binding molecule of claim 1, wherein the isolated antigen binding molecule comprises a light chain CDR2 sequence selected from the group consisting of SEQ ID NOs: 104 and 110.

21. The isolated antigen binding molecule of claim 1, wherein the isolated antigen binding molecule comprises a light chain CDR3 sequence comprising SEQ ID NO: 116.

22-45. (canceled)
